(19) United States
(12) Patent Application Publication
(10) Pub. No.: US 2026/0226463 A1
(43) Pub. Date: Aug. 6, 2026

(54) OLIGONUCLEOTIDES FOR MODULATING APOLIPOPROTEIN E (APOE) EXPRESSION AND METHODS OF USE THEREOF

(71) Applicant: SciNeuro Therapeutics Inc., Rockville, MD (US)

(72) Inventors: Min LI, Ponte Vedra Beach, FL (US); Andreas ZEMBRZYCKI, San Diego, CA (US); Mary Elizabeth CURTIS, Potomac, MD (US); Yuka MARTENS, Gaithersburg, MD (US); Dai-Shi SU, Dresher, PA (US)

(21) Appl. No.: 19/154,968

(22) PCT Filed: Feb. 13, 2024
(86) PCT No.: PCT/US2024/015653

§ 371 Date: Aug. 8, 2025

Related U.S. Application Data
(60) Provisional application No. 63/484,581, filed on Feb. 13, 2023.

(51) Int. Cl. C12N 15/113; A61K 45/06; A61P 35/00

(52) U.S. Cl. CPC C12N 15/113; A61K 45/06; A61P 35/00; C12N 2310/11; C12N 2310/3125; C12N 2310/313; C12N 2310/314; C12N 2310/315; C12N 2310/321; C12N 2310/322; C12N 2310/3231; C12N 2310/3233; C12N 2310/333; C12N 2310/334; C12N 2310/3341; C12N 2310/335; C12N 2310/336; C12N 2310/341; C12N 2320/31

(57) ABSTRACT

The present disclosure relates to oligonucleotides, in particular antisense oligonucleotides (ASOs) and pharmaceutically acceptable salts thereof, that can hybridize and reduce the expression of APOE pre-mRNA or mRNA. ASOs disclosed herein can reduce translation of APOE protein in mammals (e.g., humans). The present disclosure further relates to methods of treating a disease or disorder in a subject in need thereof by administration of an antisense oligonucleotide disclosed herein. In particular, methods and ASOs described herein can be used for preventing and/or treating human diseases in which the reduction of APOE amount or its activity would be beneficial, including but not limited to neurodegenerative diseases such as Alzheimer's disease (AD), dementia with Lewy bodies (DLB), Parkinson disease dementia (PDD), and those broadly defined as tauopathies and synucleinopathies.

### CROSS-REFERENCE TO RELATED APPLICATIONS

The present application claims the benefit of U.S. Provisional Application No. 63/484,581, filed on Feb. 13, 2023, the entire contents of which is incorporated herein by reference.

### REFERENCE TO A SEQUENCE LISTING XML

This application contains a Sequence Listing which has been submitted electronically in XML format. The Sequence Listing XML is incorporated herein by reference. Said XML file, created on Feb. 13, 2024, is named 767173_000017_SL.xml and is 403,882 bytes in size.

### BACKGROUND

Neurodegenerative diseases are caused by the progressive loss of structure or cellular function of neurons and ensuing cell death in neuronal tissues, a process known as neurodegeneration. Neurodegeneration can progress through different components or levels of neuronal circuitry, which lead to distinct clinical manifestations such as Alzheimer's disease, Parkinson's disease, multiple system atrophy, amyotrophic lateral sclerosis, Huntington's disease, multiple sclerosis, prion diseases and other human diseases.

Alzheimer's disease (AD) as a non-limiting example is the most common neurodegenerative disease and the leading cause of dementia in elderly individuals. Senile plaques consisting of amyloid-β (Aβ) fibrils, neurofibrillary tangles consisting of aggregated tau proteins, and neuronal dystrophy and neuronal cell loss characterize AD pathology and constitute its diagnostic biomarker profile (see Jack C R, Jr. et al. (2018) Alzheimers Dement 14:535-562). The earliest pathological changes may occur 20 years or more before clinical symptom onset in AD (see Jack C R, Jr. et al. Alzheimer's Disease Neuroimaging I (2009) Brain 132:1355-1365; Braak H, et al. (2011) J Neuropathol Exp Neurol 70:960-969; Bateman R J et al. (2012) N Engl J Med 367:795-804; Gordon B A et al. (2018) Lancet Neurol 17:241-250; Barthelemy N R et al. (2020) Nat Med 26:398-407; Quiroz Y T et al. (2020) Lancet Neurol 19:513-521) suggesting opportunities for early intervention and disease-modifying therapies.

AD susceptibility is influenced by common genetic variants, among which APOE allelic variant ε4 (APOE4) stands out for strong effect sizes and significant enrichment within patient populations. Relative to the risk-neutral ε3/ε3 genotype, inheritance of one or two ε4 alleles increases the risk of AD by 3-4 fold or 9-15 fold, respectively (see Farrer L A, et al. (1997) JAMA 278:1349-1356; Genin E, et al. (2011) Mol Psychiatry 16:903-907; Neu S. C., et al. (2017) JAMA Neurol 74:1178-1189). Furthermore, inheritance of one &4 allele decreases the age of AD onset by 2-8 years, and two &4 alleles decrease the age of onset by 5-16 years, compared to 84 non-carriers (see Corder E H, et al. (1993) Science 261:921-923; Sando S B, et al. (2008) BMC Neurol 8:9)). The average allele frequency of 84 was found to be 38% among patients with AD, versus 14% in cognitively healthy individuals in a meta-analysis of 38 case-control sample pairs across multiple ethnic groups (Alzgene. APOE_2/3/4. http://www.alzgene.org/meta.asp?geneID=83, updated 2010).

The human APOE protein consists of a receptor-binding region in the amino-terminal domain and the lipid-binding region in the carboxy-terminal domain. Nascent APOE protein is secreted by cells and associates with the cell surface ATP-binding cassette transporters ABCA1 and ABCG1, which transfer cholesterol and phospholipids to nascent APOE to form lipoprotein particles (see Wahrle S E, et al. (2004) J Biol Chem 279:40987-40993)

In the healthy brain, APOE is primarily expressed in astrocytes, microglia, vascular mural cells, and choroid plexus cells; however, under pathological or injury conditions, APOE expression is vastly upregulated in myeloid cells including microglia and macrophages (see Xu Q, et al. (2006) J Neurosci 26:4985-4994; Yamazaki et al, (2019) Nat Rev Neurol 15 (9): 501-518). It plays a physiological and pathophysiological role of shuttling cholesterol and other lipids among different brain cell types via cell-surface APOE receptors (see Hauser P S, et al. (2011) Prog Lipid Res 50:62-74; Yamazaki et al, (2019) Nat Rev Neurol 15 (9): 501-518).

The common APOE ε4 and ε3 allelic variants encode the APOE4 and APOE3 protein isoforms, respectively. APOE4 differs from APOE3 at amino acid position 112 (APOE3: Cys112, APOE4: Arg112). Despite only differing by a single amino acid substitution, APOE4 has a multitude of effects on AD pathogenesis pathways, see Yamazaki Y, et al. (2019) Nat Rev Neurol 15:501-518, that are often characterized as toxic gain-of function APOE effects. The best characterized of such toxic gain-of-function effects is that APOE4 reduces Aβ clearance and promotes AB aggregation, which correlates with the clinical phenotype of accelerated Aβ plaque deposition in ε4 allele carriers, with ε4/ε4 homozygous carriers showing the most accelerated deposition. In both humans and animal models, APOE4 is associated with compromised blood-brain barrier (BBB) integrity (see Ishii M and Iadecola C (2020) Nature 581:31-32; Montagne A et al. (2020) Nature 581:71-76; Jackson R J, et al. (2022) Brain 145:3582-3593)). In addition, the &4 allele is also associated with higher levels of TAR DNA-binding protein 43 (TDP-43) pathology in the brains of individuals with AD (see Vossel K A, et al. (2013) Neurocase 19:295-301; Josephs K A, et al. (2017) Lancet Neurol 16:917-924; Yang H S, et al. (2018) Lancet Neurol 17:773-781). Further, the 84 allele increases risk to develop dementia with Lewy bodies (see Tsuang D et al. (2013) JAMA Neurol 70:223-228; Bras J et al. (2014) Hum Mol Genet 23:6139-6146; Guerreiro R et al. (2018) Lancet Neurol 17:64-74) and Parkinson's disease dementia (PDD) (see Huang X, et al. (2006) Arch Neurol 63:189-193; Irwin D J, et al. (2012) Ann Neurol 72:587-598; Tsuang D et al. (2013) JAMA Neurol 70:223-228; Tropea T F, et al. (2018) Mov Disord 33:289-297).

In experimental studies, APOE4 was shown to aggravate tau pathology and tau-mediated neurodegeneration in animal models. Furthermore, APOE4 was shown to induce effects that can be characterized as reduction of APOE physiological and pathophysiological functions including microglial responsiveness, neuroinflammation, energy metabolism, cholesterol and lipid transport and homeostasis, synaptic integrity and plasticity, glucose metabolism, and neurovascular integrity and function (see Liu C C, et al. Nat Rev Neurol 9:106-118; Yamazaki Y, et al. (2019) Nat Rev Neurol 15:501-518; Martens Y A, et al. (2022) Neuron 110:1304-1317). Furthermore, APOE4 also hinders the physiological functions of APOE, such as microglial responsiveness, neuroinflammation, energy metabolism, cholesterol and lipid transport and homeostasis, synaptic integrity and plasticity, glucose metabolism, and neurovascular integrity and function (see Liu C C, et al. Nat Rev Neurol 9:106-118; Yamazaki Y, et al. (2019) Nat Rev Neurol 15:501-518; Martens Y A, et al. (2022) Neuron 110:1304-1317).

Preclinical data is available demonstrating that certain ASOs targeting APOE applied via intracerebroventricular infusion (icv) during the early stages of pathological development in AD model mice can be effective to reduce brain amyloid burden. Most importantly, this intervention protects AD mice from developing tau pathology, reduces neuritic dystrophy, and lowers levels of the neuronal damage marker neurofilament light chain (NfL) independent of amyloid pathology or timing of ASO application relative to onset of AD pathologies. Additional readouts documented that icv delivery of APOE ASO protected against tau pathology and associated neurodegeneration, preserved synaptic density (reduced synapse loss) and decreased neuroinflammatory markers and pro-inflammatory cytokine release in the brain without affecting the lipid levels in liver and plasma (see Huynh T V, et al. (2017) Neuron 96:1013-1023 e1014; Litvinchuk A. et al. (2021) Ann Neurol 89:952-966).

Recent data utilizing biochemical, in vitro and in vivo methodologies in rodent disease models suggests that certain monoclonal antibodies targeting APOE (α-APOE antibodies) that are selective for nonlipidated, aggregated APOE can preferentially bind APOE in amyloid plaques. These antibodies have a similar mode of action and efficacy compared to AB-targeting monoclonal antibodies (anti-Aβ antibodies) that also bind to and decrease Aβ aggregates via a microglial-mediated clearance mechanism (see Liao F et al. (2018) J Clin Invest 128:2144-2155). Moreover certain α-APOE antibodies appear to specifically bind dense core plaques while other anti-Aβ antibodies bind amyloids broadly, including those deposited in the brain vessels as seen in cerebral amyloid angiopathy (CAA). Mechanistically α-APOE antibodies that specifically target plaques initially increase glial activation leading to a sustained microglial recruitment and improved vascular functions whereas less specific anti-Aβ antibodies that target vascular amyloid induce chronic astrogliosis around CAA vessels and sustained CAA pathology (see Xiong M, et al. (2021) Sci Transl Med 13, 581). The resulting neurovascular pathologies and damage observed in animal models are consistent with amyloid-related imaging abnormalities (ARIA-E & ARIA-H pathology) that is observed in human subjects treated with anti-Aβ antibodies (see Sperling R A et al. (2011) Alzheimers Dement 7:367-385, 2011; Salloway S, et al. (2022) JAMA Neurol 79:13-21). A related dataset demonstrates that the seeding, spreading of tau and the resulting dystrophy in neurites are reduced following sustained treatment with α-APOE antibodies in a mouse model of AD in a manner that is consistent with the reduction of overall individual amyloid load (see Gratuze M, Jiang H, Wang C, Xiong M, Bao X, Holtzman D M (2022) Ann Neurol 91:847-852). It appears that while the potency to reduce amyloid plaques is similar between amyloid and APOE-targeting molecules, APOE-targeting molecules could be superior by mediating additional beneficial effects on vascular phenotypes while also reducing tau seeding, spreading & pathology (see Liao F et al. (2018) J Clin Invest 128:2144-2155; Xiong M, et al. (2021) Sci Transl Med 13, 581; Gratuze M, et al. (2022) Ann Neurol 91:847-852). Unfortunately, antibody therapies, such as α-APOE antibodies, can suffer from poor penetration into the brain tissue due to their large molecular weight, short retention and duration of effect in the brain following direct administration, and are limited to extracellular effects. As such, there is a need for additional agents and methods of reducing APOE expression.

### SUMMARY OF THE CLAIMS

As described herein, the current disclosure relates to antisense oligonucleotides and methods of use thereof. As such, in one aspect, the disclosure provides an antisense oligonucleotide or a pharmaceutically acceptable salt thereof comprising a sequence that is at least 80% identical to SEQ ID NO: 5-11, 15, 16, 20-41, 43, 45, 47-50, 53, 55-57, 60-75, or 77-81.

In another aspect, the disclosure provides an antisense oligonucleotide or a pharmaceutically acceptable salt thereof comprising a sequence selected from the group consisting of SEQ ID NOs: 5-11, 15, 16, 20-41, 43, 45, 47-50, 53, 55-57, 60-75, and 77-81. In certain embodiments, the antisense oligonucleotide or a pharmaceutically acceptable salt thereof has a sequence that is selected from the group consisting of SEQ ID NOs: 7, 8, 10, 15, 32, 33, 35, 36, 37, 38, 39, 40, 41, 43, 45, 49, 56, 57, 60, 61, 62, 63, 64, 65, 66, 67, 68, 69, 71, 71, 72, 73, 74, 77, 78, 79, 80, and 81.

In another aspect, the disclosure provides an antisense oligonucleotide or a pharmaceutically acceptable salt thereof comprising a sequence that is at least 80% identical to SEQ ID NO: 84, 85, 88, 90, 91, 94, 96, 100, 103, 104, 108, 111, 113, 114, 115, 119, 120, 123, 124, 129, 130, 131, 133, 134, 135, 136, 138, 150, 151, 157, 163, 164, 168, 170, 175, 176, 180, 185, 210, 212, 216, 219, 228, or 229.

In another aspect, the disclosure provides an antisense oligonucleotide or a pharmaceutically acceptable salt thereof comprising a sequence selected from the group consisting of SEQ ID NOs: 84, 85, 88, 90, 91, 94, 96, 100, 103, 104, 108, 111, 113, 114, 115, 119, 120, 123, 124, 129, 130, 131, 133, 134, 135, 136, 138, 150, 151, 157, 163, 164, 168, 170, 175, 176, 180, 185, 210, 212, 216, 219, 228, and 229. In certain embodiments, the antisense oligonucleotide or a pharmaceutically acceptable salt thereof has a sequence that is selected from the group consisting of SEQ ID NOs: 127, 132, 147, 202, and 220.

In certain embodiments, the antisense oligonucleotide or pharmaceutically acceptable salt thereof comprises about 15 to about 25 nucleobases. In certain embodiments, the antisense oligonucleotide or pharmaceutically acceptable salt thereof comprises about 18 to about 22 nucleobases. In certain embodiments, the antisense oligonucleotide or pharmaceutically acceptable salt thereof comprises about 20 nucleobases. In certain embodiments, the antisense oligonucleotide or pharmaceutically acceptable salt thereof comprises about 18 nucleobases.

In certain embodiments, the antisense oligonucleotide or pharmaceutically acceptable salt thereof comprises about 12 to about 25 nucleobases. In certain embodiments, the antisense oligonucleotide or pharmaceutically acceptable salt thereof comprises about 14 to about 18 nucleobases. In certain embodiments, the antisense oligonucleotide or pharmaceutically acceptable salt thereof comprises about 16 nucleobases.

In certain embodiments, the antisense oligonucleotide comprises one or more inter-nucleoside linkage modifications. The inter-nucleoside linkage modification can be selected from the group consisting of a phosphorothioate, a phosphorodithioate, a methyl phosphonate, a methyl phosphorothioate, a phosphoramidate, a phosphorodiamidate, a thiophosphoramidate, a mesyl phosphoramidate, and a combination thereof. In certain embodiments, the antisense oligonucleotide comprises one or more phosphorothioate inter-nucleoside linkages. In certain embodiments, all of the inter-nucleoside linkages are phosphorothioate linkages.

In certain embodiments, the antisense oligonucleotide comprises DNA. In certain embodiments, the antisense oligonucleotide comprises RNA. In certain embodiments, the antisense oligonucleotide comprises a protein and/or polypeptide. In certain embodiments, the antisense oligonucleotide comprises a small molecule.

In certain embodiments, the antisense oligonucleotide comprises one or more modified nucleosides. The modified nucleoside can be selected from the group consisting of a 2′-modified nucleoside, a 4′-modified nucleoside, a bridged nucleoside, a phosphorodiamidate morpholine, a locked nucleic acid, an ethylene-bridged nucleic acid, a glycol nucleic acid, a hexitol nucleic acid, a cyclohexene nucleic acid, an arabino nucleic acid, a peptide nucleic acid, a threose nucleic acid, a tricyclo-2′-deoxy-nucleotide, a 1′-deoxyribopentose, a 1′,2′-dideoxyribopentose, 2′,3′-dideoxyribopentose, 2′,3′-didehydro-2′,3′-dideoxyribopentose, an unlocked nucleic acid, and a combination thereof. The antisense oligonucleotide can comprise one or more 2′-nucleoside modifications. The antisense oligonucleotide can comprise at least eight 2′-nucleoside modifications. The antisense oligonucleotide can comprise at least ten 2′-nucleoside modifications.

In certain embodiments, all nucleosides of the antisense oligonucleotide comprise a 2′-nucleoside modification. The 2′-nucleoside modification can be selected from the group consisting of a 2′-fluoro-nucleoside, a 2′-O-methyl-nucleoside, a 2′-O-methoxy ethyl nucleoside, a 2′-O-benzyl-2′-deoxynucleoside, a 2′-O-methyl-4-pyridinylnucleoside (2′q2Py(4)), a 2′-amino-nucleoside, and a combination thereof.

In certain embodiments, the antisense oligonucleotide comprises one or more modified nucleobases. The modified nucleobase can be a methylated nucleobase. The methylated nucleobase can be selected from the group consisting of 5-methyluracil, N6-methyladenine, N4-methylcytosine, N7-methylguanine, 5-hydroxymethylcytosine, N3-methylcytosine, and a combination thereof. The modified nucleobase can be an acetylated nucleobase. The acetylated nucleobase can be a N4-acetylcytosine. The modified nucleobase can be selected from the group consisting of pseudouridine (Ψ), N1-methyl-pseudouridine (N1-methyl-Ψ′), 2-thiouridine (s2U), 5-fluoro-2′-deoxyuridine (FUDR), 8-oxo-7,8-dihydroguanosine (8-oxoG), N-ethylpiperidine-7-EAA triazole modified adenine, N-ethylpiperidine-6-triazole modified adenine, 6-phenylpyrrolo-cytosine (PhpC), 2,4-difluorotoluyl-ribonucleoside (IF), N1 (5-nitroindole) ribonucleoside, 5-methoxyuridine), and a combination thereof.

In certain embodiments, the antisense oligonucleotide or pharmaceutically acceptable salt thereof further comprises an inverted nucleotide.

In certain embodiments, the antisense oligonucleotide is a gapmer. The gapmer can comprise flanking RNA nucleotides on the 5′-end, the 3′-end, or both. The flanking RNA nucleotides on the 5′-end and/or 3′-end can independently range from 1 nucleotide to about 9 nucleotides. The gapmer can range from about 18 to about 22 nucleosides in length. The gapmer can be 18 nucleosides in length. The gapmer can comprise ten central 2′-deoxynucleosides and four flanking RNA nucleotides each on both the 5′-end and the 3′-end. The gapmer can be 20 nucleosides in length. The gapmer can comprise ten central 2′-deoxynucleosides and/or five flanking ribonucleosides each on both the 5′-end and the 3′-end.

In certain embodiments, the gapmer ranges from about 14 to about 22 nucleosides in length. The flanking nucleotides on the 5′-end and/or 3′-end independently range from about 1 nucleotide to about 5 nucleotides. The gapmer can comprise ten central 2′-deoxynucleotides and/or three flanking RNA nucleotides each on both the 5′-end and the 3′-end. The gapmer can be 16 nucleosides in length.

In certain embodiments, administration of the antisense oligonucleotide to a subject reduces mRNA, pre-mRNA, protein expression, or a combination thereof of human APOE. Human APOE pre-mRNA or mRNA can be reduced by about 10% or greater. Human APOE pre-mRNA or mRNA can be reduced by about 30% or greater. Human APOE pre-mRNA or mRNA can be reduced by about 50% or greater. Human APOE mRNA can be reduced by about 25% or greater. Human APOE mRNA can be reduced by about 50% or greater. Human APOE mRNA can be reduced by about 70% or greater. Human APOE protein can be reduced by about 25% or greater. Human APOE protein can be reduced by about 50% or greater. Human APOE protein can be reduced by about 70% or greater.

In certain embodiments, the antisense oligonucleotide can hybridize with human APOE mRNA. The human APOE mRNA can comprise a sequence that is at least 80% identical to SEQ ID NO: 1. The human APOE mRNA can encode for a protein comprising a sequence that is 85% identical to SEQ ID NO: 82. The APOE can be an isoform selected from the group consisting of APOE 2, APOE 3, APOE 4, APOE 5f, APOE 5s, and APOE 7. The hybridization can be by Watson-Crick base pairing.

In certain embodiments, the antisense oligonucleotide can hybridize with human APOE pre-mRNA. The human APOE pre-mRNA can comprise a sequence that is at least 80% identical to SEQ ID NO: 230. The human APOE mRNA can encode for a protein comprising a sequence that is 85% identical to SEQ ID NO: 82. The APOE can be an isoform selected from the group consisting of APOE 2, APOE 3, APOE 4, APOE 5f, APOE 5s, and APOE 7. The hybridization can be by Watson-Crick base pairing.

In another aspect, the disclosure relates to a method of treating a disease or disorder in a human subject in need thereof comprising administering an antisense oligonucleotide or pharmaceutically acceptable salt thereof as described herein to a subject in need thereof.

In another aspect, the disclosure relates to a method of treating a neurodegenerative disease comprising administering an antisense oligonucleotide or pharmaceutically acceptable salt thereof as described herein to a subject in need thereof. The neurodegenerative disease can be selected from the group consisting of, but not limited to, Alzheimer's disease, amyotrophic lateral sclerosis, Huntington's disease, Lewy body dementia, multiple sclerosis, multiple system atrophy, Parkinson's disease, prion disease, and a combination thereof. The neurodegenerative disease can be Alzheimer's disease. The neurodegenerative disease can be a tauopathy. The tauopathy can be selected from the group consisting of, but not limited to, primary age-related tauopathy, chronic traumatic encephalopathy, progressive supranuclear palsy, corticobasal degeneration, frontotemporal dementia and parkinsonism linked to chromosome 17, vacuolar tauopathy, lytico-bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, behavioral variant frontotemporal dementia (Pick's disease), argyrophilic grain disease, and a combination thereof.

In another aspect, the disclosure relates to a method of treating an acute or chronic injury comprising administering an antisense oligonucleotide or pharmaceutically acceptable salt thereof as described herein to a subject in need thereof. The acute or chronic injury can be selected from the group consisting of, but not limited to, traumatic brain injury, spinal cord injury, chronic traumatic encephalopathy, chemo brain, and a combination thereof.

In another aspect, the disclosure relates to a method of treating an acute or chronic inflammatory condition comprising administering an antisense oligonucleotide or pharmaceutically acceptable salt thereof as described herein to a subject in need thereof. The acute or chronic inflammatory condition can be selected from the group consisting of, but not limited to, stroke, multiple sclerosis, infectious disease, immune response to cancer development and a combination thereof.

In another aspect, the disclosure relates to a method of treating a vascular disease or a vascular pathological condition comprising administering an antisense oligonucleotide or pharmaceutically acceptable salt thereof as described herein to a subject in need thereof. The vascular disease or the vascular pathological condition can be selected from the group consisting of, but not limited to, cerebral small vessel disease, blood-brain barrier leakage, atherosclerosis, cerebral amyloid angiopathy and a combination thereof.

In another aspect, the disclosure relates to a method of treating a lipid storage disorder comprising administering an antisense oligonucleotide or pharmaceutically acceptable salt thereof as described herein to a subject in need thereof. The lipid storage disorder can be selected from the group consisting of, but not limited to, Cerebrotendineous xanthomatosis, Farber disease, Fabry disease, fucosidosis, Gaucher disease, GM1 gangliosidoses, GM2-gangliosidosis AB variant, Krabbe disease, metachromatic leukodystrophy, multiple sulfatase deficiency, Niemann Pick disease type A, Niemann Pick disease type B, Niemann-Pick disease type C, Sandhoff disease, Schindler disease, Tay-Sachs disease, Wolman disease, and a combination thereof.

In another aspect, the disclosure relates to use of an antisense oligonucleotide or pharmaceutically acceptable salt thereof as described herein for the manufacture of a medicament for the therapeutic treatment of a disease or disorder in a human subject in need thereof.

In another aspect, the disclosure relates to use of an antisense oligonucleotide or pharmaceutically acceptable salt thereof as described herein for treatment of a disease or disorder in a human subject in need thereof.

In another aspect, the disclosure relates to use of an antisense oligonucleotide or pharmaceutically acceptable salt thereof as described herein for the manufacture of a medicament for the therapeutic treatment of a neurodegenerative disease in a subject in need thereof.

In another aspect, the disclosure relates to use of an antisense oligonucleotide or pharmaceutically acceptable salt thereof as described herein for treatment of a neurodegenerative disease in a subject in need thereof. The neurodegenerative disease can be selected from the group consisting of, but not limited to, Alzheimer's disease, amyotrophic lateral sclerosis, Huntington's disease, Lewy body dementia, multiple sclerosis, multiple system atrophy, Parkinson's disease, prion disease, and a combination thereof. The neurodegenerative disease can be a tauopathy. The tauopathy can be selected from the group consisting of primary age-related tauopathy, chronic traumatic encephalopathy, progressive supranuclear palsy, corticobasal degeneration, frontotemporal dementia and parkinsonism linked to chromosome 17, vacuolar tauopathy, lytico-bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, behavioral variant frontotemporal dementia (Pick's disease), argyrophilic grain disease, and a combination thereof.

In certain embodiments, the oligonucleotide or pharmaceutically acceptable salt thereof is administered locally to a subject in need thereof. The local administration can be a intracranial, intracerebral, a intramuscular, a spinal, a epidural, sacroiliac, or a subdural injection.

In certain embodiments, the oligonucleotide or pharmaceutically acceptable salt thereof is administered systemically to a subject in need thereof.

### BRIEF DESCRIPTION OF THE DRAWING

FIGS. 1A-1C are phase contrast microscopy images showing that some ASOs (e.g. S1038 (SEQ ID NO: 24)) result in abnormal morphological cell changes, while others result in comparable morphology to untreated control in HEP3B cells. All cells were cultured for 48 hours with or without ASO. FIG. 1A is a microscopy image showing HEP3B cells untreated with ASO (“Untreated control”). FIG. 1B is a microscopy image showing that HEP3B cells treated with ASO S1038 (12.5 nM (SEQ ID NO: 24)) display an abnormal epithelial cell morphology (e.g., loss of confluency, dense pyknotic nuclei) compared to untreated HEP3B cells, indicative of cellular toxicity and death. FIG. 1C is a microscopy image showing that HEP3B cells treated with ASO S1094 (12.5 nM (SEQ ID NO: 80)) display comparable morphology and confluency to HEP3B cells untreated with ASO. This indicates the ASO S1094 (SEQ ID NO: 80) is non-toxic to HEP3B cells.

### DETAILED DESCRIPTION OF THE INVENTION

Apolipoprotein E (APOE) mutations and expression have been highly correlated with human disease, especially neurological diseases (e.g., Alzheimer's disease (AD), dementia with Lewy bodies, Parkinson's disease). AD susceptibility is influenced by common genetic variants, among which APOE allelic variant ε4 (APOE4) stands out for strong effect sizes and significant enrichment within patient populations. Relative to the risk-neutral ε3/ε3 genotype, inheritance of one or two ε4 alleles increases the risk of AD by 3-4 fold or 9-15 fold, respectively (see Farrer L A, et al. (1997) JAMA 278:1349-1356; Genin E, et al. (2011) Mol Psychiatry 16:903-907; Neu S. C., et al. (2017) JAMA Neurol 74:1178-1189). Accordingly, APOE has been investigated as a potential target for the treatment of neurological diseases and other disease where APOE may play a role in disease pathology. Unfortunately, antibody therapies, such as α-APOE antibodies, can suffer from poor penetration into the brain tissue due to their large molecular weight, short retention and duration of effect in the brain following direct administration, and are limited to extracellular effects. As such, there is a need for additional agents and methods of reducing APOE expression.

### Antisense Oligonucleotides

This disclosure relates to antisense oligonucleotides and pharmaceutically related salts thereof that target (e.g., hybridize with, Watson-Crick base-pair with, bind to, reduce the expression of) APOE mRNA and/or pre-mRNA. In certain embodiments of the present disclosure, an antisense oligonucleotide or a pharmaceutically acceptable salt thereof comprises a sequence that is at least about 80%, 85%, 90%, 91%, 92%, 93%, 94%, 95%, 96%, 97%, 98%, 99%, or 100% identical to at least one of SEQ ID NO: 5-11, 15, 16, 20-41, 43, 45, 47-50, 53, 55-57, 60-75, or 77-81. The antisense oligonucleotide or a pharmaceutically acceptable salt thereof can comprise at least one of SEQ ID NOs: 5-11, 15, 16, 20-41, 43, 45, 47-50, 53, 55-57, 60-75, or 77-81. In certain embodiments, the antisense oligonucleotide or a pharmaceutically acceptable salt thereof comprises at least one of SEQ ID NOs: 7, 8, 10, 15, 32, 33, 35, 36, 37, 38, 39, 40, 41, 43, 45, 49, 56, 57, 60, 61, 62, 63, 64, 65, 66, 67, 68, 69, 71, 71, 72, 73, 74, 77, 78, 79, 80, or 81.

In certain embodiments of the present disclosure, an antisense oligonucleotide or a pharmaceutically acceptable salt thereof comprises a sequence that is at least about 80%, 85%, 90%, 91%, 92%, 93%, 94%, 95%, 96%, 97%, 98%, 99%, or 100% identical to at least one of SEQ ID NOs: 3-81. In certain embodiments of the present disclosure, an antisense oligonucleotide or a pharmaceutically acceptable salt thereof comprises at least one of SEQ ID NOs: 3-81. In certain embodiments of the present disclosure, an antisense oligonucleotide or a pharmaceutically acceptable salt thereof comprises a sequence that is at least about 80%, 85%, 90%, 91%, 92%, 93%, 94%, 95%, 96%, 97%, 98%, 99%, or 100% identical to at least one of SEQ ID NOs: 7, 8, 10, 15, 32, 33, 35, 36, 37, 38, 39, 40, 41, 43, 45, 49, 56, 57, 60, 61, 62, 63, 64, 65, 66, 67, 68, 69, 71, 71, 72, 73, 74, 77, 78, 79, 80, or 81.

The target APOE oligonucleotide sequence can be pre-mRNA. Pre-mRNA is the result of DNA transcription into pre-mRNA. Following transcription, the pre-mRNA undergoes processing steps, such as addition of a 5′-cap, addition of a poly-A tail, and splicing out of introns, resulting in mature mRNA. The target pre-mRNA can be pre-mRNA encoding any APOE isoform, such as APOE 2, APOE 3, APOE 4, APOE 5f, APOE 5s, or APOE 7. For example, the target can be APOE pre-mRNA having a nucleic acid sequence according to SEQ ID NO: 230. In certain embodiments of the present disclosure, an antisense oligonucleotide or a pharmaceutically acceptable salt thereof comprises a sequence that is at least about 80%, 85%, 90%, 91%, 92%, 93%, 94%, 95%, 96%, 97%, 98%, 99%, or 100% identical to at least one of SEQ ID NOs: 84, 85, 88, 90, 91, 94, 96, 100, 103, 104, 108, 111, 113, 114, 115, 119, 120, 123, 124, 129, 130, 131, 133, 134, 135, 136, 138, 150, 151, 157, 163, 164, 168, 170, 175, 176, 180, 185, 210, 212, 216, 219, 228, or 229. In certain embodiments of the present disclosure, an antisense oligonucleotide or a pharmaceutically acceptable salt thereof comprises a sequence that is at least about 80%, 85%, 90%, 91%, 92%, 93%, 94%, 95%, 96%, 97%, 98%, 99%, or 100% identical to at least one of SEQ ID NOs: 84, 119, 120, 123, 124, 129, 133-136, 168, 185, 228, or 229. In certain embodiments of the present disclosure, an antisense oligonucleotide or a pharmaceutically acceptable salt thereof comprises a sequence that is at least about 80%, 85%, 90%, 91%, 92%, 93%, 94%, 95%, 96%, 97%, 98%, 99%, or 100% identical to at least one of SEQ ID NOs: 84-229. In certain embodiments of the present disclosure, an antisense oligonucleotide or a pharmaceutically acceptable salt thereof comprises a sequence that is at least about 80%, 85%, 90%, 91%, 92%, 93%, 94%, 95%, 96%, 97%, 98%, 99%, or 100% identical to at least one of SEQ ID NOs: 127, 132, 147, 202, or 220. The antisense oligonucleotide or a pharmaceutically acceptable salt thereof can comprise at least one of SEQ ID NOs: 84, 85, 88, 90, 91, 94, 96, 100, 103, 104, 108, 111, 113, 114, 115, 119, 120, 123, 124, 129, 130, 131, 133, 134, 135, 136, 138, 150, 151, 157, 163, 164, 168, 170, 175, 176, 180, 185, 210, 212, 216, 219, 228, or 229. In certain embodiments, the antisense oligonucleotide or a pharmaceutically acceptable salt thereof can comprise at least one of SEQ ID NOs: 84, 119, 120, 123, 124, 129, 133-136, 168, 185, 228, or 229. In certain embodiments, the antisense oligonucleotide or a pharmaceutically acceptable salt thereof comprises at least one of SEQ ID NOs: 127, 132, 147, 202, or 220. In certain embodiments, the antisense oligonucleotide or a pharmaceutically acceptable salt thereof comprises at least one of SEQ ID NOs: 84-229.

The antisense oligonucleotide or pharmaceutically acceptable salt thereof comprises about 15 to about 25 nucleobases, about 15 to about 24 nucleobases, about 15 to about 23 nucleobases, about 15 to about 22 nucleobases, about 15 to about 21 nucleobases, about 15 to about 20 nucleobases, about 15 to about 19 nucleobases, about 15 to about 18 nucleobases, about 18 to 22 nucleobases, about 18 to about 21 nucleobases, about 18 to about 20 nucleobases, about 15 nucleobases, about 16 nucleobases, about 17 nucleobases, about 18 nucleobases, about 19 nucleobases, about 20 nucleobases, about 21 nucleobases, or about 22 nucleobases. In some embodiments the antisense oligonucleotide or pharmaceutically acceptable salt thereof can comprise about 12 to about 25 nucleobases, about 13 to about 25 nucleobases, about 14 to about 25 nucleobases, about 12 to about 24 nucleobases, about 12 to about 23 nucleobases, about 12 to about 22 nucleobases, about 12 to about 21 nucleobases, about 12 to about 20 nucleobases, about 12 to about 19 nucleobases, about 12 to about 18 nucleobases, about 12 to about 17 nucleobases, about 12 to about 16 nucleobases, about 12 to about 15 nucleobases, about 12 to about 14 nucleobases, about 12 to about 13 nucleobases, about 14 to about 24 nucleobases, about 14 to about 23 nucleobases, about 14 to about 22 nucleobases, about 14 to about 21 nucleobases, about 14 to about 20 nucleobases, about 14 to about 19 nucleobases, about 14 to about 18 nucleobases, about 14 to about 17 nucleobases, about 14 to about 16 nucleobases, about 14 to about 15 nucleobases, about 12 nucleobases, about 13 nucleobases, or about 14 nucleobases. The antisense oligonucleotide or pharmaceutically acceptable salt thereof can comprise about 20 nucleobases. The antisense oligonucleotide or pharmaceutically acceptable salt thereof can comprise about 18 nucleobases. The antisense oligonucleotide or pharmaceutically acceptable salt thereof can comprise about 16 nucleobases.

In certain embodiments, the antisense oligonucleotide can comprise one or more inter-nucleoside linkage modifications. The inter-nucleoside linkage modification can be a phosphorothioate, a phosphorodithioate, a methyl phosphonate, a methyl phosphorothioate, a phosphoramidate, a phosphorodiamidate, a thiophosphoramidate, a mesyl phosphoramidate, or a combination thereof. The antisense oligonucleotide can comprise one or more phosphorothioate inter-nucleoside linkages. In certain embodiments, all of the inter-nucleoside linkages are phosphorothioate linkages.

The antisense oligonucleotide can comprise DNA, RNA, a protein and/or polypeptide. In certain embodiments, the antisense oligonucleotide comprises a small molecule.

In certain embodiments, the antisense oligonucleotide can comprise one or more modified nucleosides. The modified nucleoside can be a 2′-modified nucleoside, a 4′-modified nucleoside, a bridged nucleoside, a phosphorodiamidate morpholine, a locked nucleic acid, an ethylene-bridged nucleic acid, a glycol nucleic acid, a hexitol nucleic acid, a cyclohexene nucleic acid, an arabino nucleic acid, a peptide nucleic acid, a threose nucleic acid, a tricyclo-2′-deoxy-nucleotide, a 1′-deoxyribopentose, a 1′,2′-dideoxyribopentose, 2′,3′-dideoxyribopentose, 2′,3′-didehydro-2′,3′-dideoxyribopentose, an unlocked nucleic acid, or a combination thereof. The antisense oligonucleotide can comprise one or more 2′-nucleoside modifications. The antisense oligonucleotide can comprise at least 1, 2, 3, 4, 5, 6, 7, 8, 9, or 10 2′-nucleoside modifications. The antisense oligonucleotide can comprise at least eight 2′-nucleoside modifications. The antisense oligonucleotide can comprise at least ten 2′-nucleoside modifications.

In certain embodiments, all nucleosides of the antisense oligonucleotide comprise a 2′-nucleoside modification. The 2′-nucleoside modification can be a 2′-fluoro-nucleoside, a 2′-O-methyl-nucleoside, a 2′-O-methoxy ethyl nucleoside, a 2′-O-benzyl-2′-deoxynucleoside, a 2′-O-methyl-4-pyridinylnucleoside (2′q2Py(4)), a 2′-amino-nucleoside, or a combination thereof.

In certain embodiments, the antisense oligonucleotide can comprise one or more modified nucleobases (e.g., 1, 2, 3, 4, 5, 6, 7, 8, 9, or 10 modified nucleobases). The modified nucleobase can be a methylated nucleobase. The modified nucleobase can be 5-methyluracil, N6-methyladenine, N4-methylcytosine, N7-methylguanine, 5-hydroxymethylcytosine, N3-methylcytosine, or a combination thereof. The modified nucleobase can be an acetylated nucleobase. The acetylated nucleobase can be a N4-acetylcytosine. The modified nucleobase can be pseudouridine (Ψ), N1-methyl-pseudouridine (N1-methyl-Ψ), 2-thiouridine (s2U), 5-fluoro-2′-deoxyuridine (FUDR), 8-oxo-7,8-dihydroguanosine (8-oxoG), N-ethylpiperidine-7-EAA triazole modified adenine, N-ethylpiperidine-6-triazole modified adenine, 6-phenylpyrrolo-cytosine (PhpC), 2,4-difluorotoluyl-ribonucleoside (rF), N1 (5-nitroindole) ribonucleoside, 5-methoxyuridine), or a combination thereof.

In certain embodiments, the antisense oligonucleotide or pharmaceutically acceptable salt thereof further comprises an inverted nucleotide.

In certain embodiments, the antisense oligonucleotide is a gapmer. The gapmer can comprise flanking RNA nucleotides on the 5′-end, the 3′-end, or both. The flanking RNA nucleotides on the 5′-end and/or 3′-end can independently range from about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, or about 1 to about 3 nucleotides. The gapmer can range from about 18 to about 22 nucleosides in length (e.g, about 18, 19, 20, 21, or 22 nucleosides). The gapmer can be 18 nucleosides in length. The gapmer can comprise about ten central 2′-deoxynucleosides and about four flanking RNA nucleotides each on both the 5′-end and the 3′-end. The gapmer can be 20 nucleosides in length. The gapmer can comprise about ten central 2′-deoxynucleosides and/or about five flanking ribonucleosides each on both the 5′-end and the 3′-end.

The gapmer can be of any length. The length of the gapmer nucleotides or nucleosides can be about 14 to about 22, about 15 to about 22, about 16 to about 22, about 17 to about 22, abut 18 to about 22, about 19 to about 22, about 20 to about 22, about 21 to about 22, about 14 to about 21, about 14 to about 20, about 14 to about 19, about 14 to about 18, about 14 to about 17, about 14 to about 16, or about 14 to about 15. The length of the gapmer nucleotides or nucleosides can be about 14, about 15, about 16, about 17, about 18, about 19, about 20, about 21, or about 22. The gapmer can comprise about ten central 2′-deoxynucleosides and about three flanking RNA nucleotides each on both the 5′-end and the 3′-end. The gapmer can comprise about ten central 2′-deoxynucleosides and/or about three flanking RNA nucleotides each on both the 5′-end and the 3′-end. In certain embodiments, the gapmer is 16 nucleosides in length. In certain embodiments, the gapmer is 18 nucleosides in length. In certain embodiments, the gapmer ranges from about 14 to about 22 nucleosides in length. In certain embodiments, the flanking RNA nucleotides on the 5′-end and/or 3′-end independently range from about 1 nucleotide to about 5 nucleotides.

The gapmer can have any combination of lengths of nucleotides and number of flanking nucleotides (e.g., 3′-end flanking nucleotides, 5′-end flanking nucleotides, or both) described herein. The flanking nucleotides (3′-end flanking nucleotides, 5′-end flanking nucleotides) can contain any number of modified nucleotides or nucleosides. The modified nucleoside or nucleotide can be modified in any way, such as any of the modifications described herein (e.g., 2′-modification, locked nucleic acid modification). The modified nucleosides or nucleotides within a flanking region can be consecutive. The modified nucleosides or nucleotides within a flanking region can be non-consecutive (e.g., a non-modified nucleotide can exist between two or more modified nucleotides in a flanking region). The gapmer can contain 14 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-12-1, 1-11-2, 2-11-1, 3-10-1, 1-10-3, 2-10-2, 3-9-2, 2-9-3, 1-9-4, 4-9-1, 5-8-1, 1-8-5, 4-8-2, 2-8-4, or 3-8-3, wherein the values X-Y-Z indicate the 5′-end flanking nucleotides-central nucleotides- and 3-flanking nucleotides. The gapmer can contain 15 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-13-1, 1-12-2, 2-12-1, 3-11-1, 1-11-3, 2-11-2, 3-10-2, 2-10-3, 1-10-4, 4-10-1, 5-9-1, 1-9-5, 4-9-2, 2-9-4, 3-9-3, 4-8-3, 3-8-4, 2-8-5, 5-8-2, 1-8-6, or 6-8-1. The gapmer can contain 16 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-14-1, 1-13-2, 2-13-1, 3-12-1, 1-12-3, 2-12-2, 3-11-2, 2-11-3, 1-11-4, 4-11-1, 5-10-1, 1-10-5, 4-10-2, 2-10-4, 3-10-3, 4-9-3, 3-9-4, 2-9-5, 5-9-2, 1-9-6, 6-9-1, 7-8-1, 1-8-7, 6-8-2, 2-8-6, 5-8-3, 3-8-5, or 4-8-4. The gapmer can contain 17 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-15-1, 1-14-2, 2-14-1, 3-13-1, 1-13-3, 2-13-2, 3-12-2, 2-12-3, 1-12-4, 4-12-1, 5-11-1, 1-11-5, 4-11-2, 2-11-4, 3-11-3, 4-10-3, 3-10-4, 2-10-5, 5-10-2, 1-10-6, 6-10-1, 7-9-1, 1-9-7, 6-9-2, 2-9-6, 5-9-3, 3-9-5, 4-9-4, 8-8-1, 1-8-8, 7-8-2, 2-8-7, 6-8-3, 3-8-6, 5-8-4, or 4-8-5. The gapmer can contain 18 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-16-1, 1-15-2, 2-15-1, 3-14-1, 1-14-3, 2-14-2, 3-13-2, 2-13-3, 1-13-4, 4-13-1, 5-12-1, 1-12-5, 4-12-2, 2-12-4, 3-12-3, 4-11-3, 3-11-4, 2-11-5, 5-11-2, 1-11-6, 6- 11-1, 7-10-1, 1-10-7, 6-10-2, 2-10-6, 5-10-3, 3-10-5, 4-10-4, 8-9-1, 1-9-8, 7-9-2, 2-9-7, 6-9- 3, 3-9-6, 5-9-4, 4-9-5, 9-8-1, 1-8-9, 8-8-2, 2-8-8, 7-8-3, 3-8-7, 6-8-4, 4-8-6, or 5-8-5. The gapmer can contain 19 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-17-1, 1-16-2, 2-16-1, 3-15-1, 1-15-3, 2-15-2, 3-14-2, 2-14-3, 1-14-4, 4-14-1, 5-13-1, 1-13-5, 4-13-2, 2-13-4, 3-13-3, 4-12-3, 3-12-4, 2-12-5, 5-12-2, 1-12-6, 6-12-1, 7-11-1, 1-11-7, 6-11-2, 2-11-6, 5-11-3, 3-11-5, 4-11-4, 8-10-1, 1-10-8, 7-10-2, 2-10-7, 6-10-3, 3-10-6, 5- 10-4, 4-10-5, 9-9-1, 1-9-9, 8-9-2, 2-9-8, 7-9-3, 3-9-7, 6-9-4, 4-9-6, 5-9-5, 10-8-1, 1-8-10, 9-8-2, 2-8-9, 8-8-3, 3-8-8, 7-8-4, 4-8-7, 5-8-6, or 6-8-5. The gapmer can contain 20 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-18-1, 1-17-2, 2-17-1, 3-16-1, 1-16-3, 2-16-2, 3-15-2, 2-15-3, 1-15-4, 4-15-1, 5-14-1, 1-14-5, 4-14-2, 2-14-4, 3-14-3, 4-13-3, 3-13-4, 2-13-5, 5-13-2, 1-13-6, 6-13-1, 7-12-1, 1-12-7, 6-12-2, 2-12-6, 5-12-3, 3- 12-5, 4-12-4, 8-11-1, 1-11-8, 7-11-2, 2-11-7, 6-11-3, 3-11-6, 5-11-4, 4-11-5, 9-10-1, 1-10-9, 8-10-2, 2-10-8, 7-10-3, 3-10-7, 6-10-4, 4-10-6, 5-10-5, 10-9-1, 1-9-10, 9-9-2, 2-9-9, 8-9-3, 3-9-8, 7-9-4, 4-9-7, 5-9-6, 6-9-5, 11-8-1, 1-8-11, 10-8-2, 2-8-10, 9-8-3, 3-8-9, 8-8-4, 4-8-8, 7-8-5, 5- 8-7, or 6-8-6. The gapmer can contain 21 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-19-1, 1-18-2, 2-18-1, 3-17-1, 1-17-3, 2-17-2, 3-16-2, 2-16-3, 1-16-4, 4-16-1, 5-15-1, 1-15-5, 4-15-2, 2-15-4, 3-15-3, 4-14-3, 3-14-4, 2-14-5, 5- 14-2, 1-14-6, 6-14-1, 7-13-1, 1-13-7, 6-13-2, 2-13-6, 5-13-3, 3-13-5, 4-13-4, 8-12-1, 1-12-8, 7-12-2, 2-12-7, 6-12-3, 3-12-6, 5-12-4, 4-12-5, 9-11-1, 1-11-9, 8-11-2, 2-11-8, 7-11-3, 3-11-7, 6-11-4, 4-11-6, 5-11-5, 10-10-1, 1-10-10, 9-10-2, 2-10-9, 8-10-3, 3-10-8, 7-10-4, 4-10-7, 5-10-6, 6-10-5, 11-9-1, 1-9-11, 10-9-2, 2-9-10, 9-9-3, 3-9-9, 8-9-4, 4-9-8, 7-9-5, 5-9-7, 6-9-6, 12-8-1, 1-8- 12, 11-8-2, 2-8-11, 10-8-3, 3-8-10, 4-8-9, 9-8-4, 8-8-5, 5-8-8, 7-8-6, or 6-8-7. The gapmer can contain 22 nucleotides and the 5′-end and/or 3′-end can independently contain about 1 to about 10 nucleotides, about 1 nucleotide to about 9 nucleotides, about 1 to about 8 nucleotides, about 1 to about 7 nucleotides, about 1 to about 6 nucleotides, about 1 to about 5 nucleotides, about 1 to about 4 nucleotides, about 1 to about 3 nucleotides, or about 1 to about 2 nucleotides. For example, the gapmer can be 1-20-1, 1-19-2, 2-19-1, 3-18-1, 1-18-3, 2-18-2, 3-17-2, 2-17-3, 1-17-4, 4-17-1, 5-16-1, 1-16-5, 4-16-2, 2-16-4, 3-16-3, 4-15-3, 3-15-4, 2-15-5, 5-15-2, 1-15-6, 6-15-1, 7-14-1, 1-14-7, 6-14-2, 2-14-6, 5-14-3, 3-14-5, 4-14-4, 8-13-1, 1-13-8, 7-13-2, 2-13-7, 6-13-3, 3-13-6, 5- 13-4, 4-13-5, 9-12-1, 1-12-9, 8-12-2, 2-12-8, 7-12-3, 3-12-7, 6-12-4, 4-12-6, 5-12-5, 10-11-1, 1-11-10, 9-11-2, 2-11-9, 8-11-3, 3-11-8, 7-11-4, 4-11-7, 5-11-6, 6-11-5, 11-10-1, 1-10-11, 10-10-2, 2- 10-10, 9-10-3, 3-10-9, 8-10-4, 4-10-8, 7-10-5, 5-10-7, 6-10-6, 12-9-1, 1-9-12, 11-9-2, 2-9-11, 10-9-3, 3-9-10, 4-9-9, 9-9-4, 8-9-5, 5-9-8, 7-9-6, 6-9-7, 13-8-1, 1-8-13, 12-8-2, 2-8-12, 11-8- 3, 3-8-11, 4-8-10, 10-8-4, 9-8-5, 5-8-9, 8-8-6, 6-8-8, or 7-8-7.

In certain embodiments, administration of the antisense oligonucleotide to a subject reduces mRNA, pre-mRNA, protein expression, or a combination thereof of human APOE. Human APOE mRNA can be reduced by about 25%, 30%, 35%, 40%, 45%, 50%, 55%, 60%, 65%, 70%, 75%, 80%, 85%, 90% or greater. In certain embodiments, administration of the antisense oligonucleotide to a subject reduces mRNA, pre-mRNA, protein expression, or a combination thereof of human APOE. Human APOE pre-mRNA or mRNA can be reduced by about 5%, 10%, 15%, 20%, 25%, 30%, 35%, 40%, 45%, 50%, 55%, 60%, 65%, 70%, 75%, 80%, 85%, 90% or greater. Human APOE pre-mRNA can be reduced by about 5%, 10%, 15%, 20%, 25%, 30%, 35%, 40%, 45%, 50%, 55%, 60%, 65%, 70%, 75%, 80%, 85%, 90% or greater. Human APOE pre-mRNA or mRNA can be reduced by about 10% or greater. Human APOE pre-mRNA or mRNA can be reduced by about 30% or greater. Human APOE pre-mRNA or mRNA can be reduced by about 50% or greater. Human APOE mRNA can be reduced by about 25% or greater. Human APOE mRNA can be reduced by about 50% or greater. Human APOE mRNA can be reduced by about 70% or greater. Human APOE protein can be reduced by about 25% or greater. Human APOE protein can be reduced by about 50% or greater. Human APOE protein can be reduced by about 70% or greater.

In certain embodiments, the antisense oligonucleotide can hybridize with human APOE mRNA. The human APOE mRNA can comprise a sequence that is at least 80%, 85%, 87%, 88%, 89%, 90%, 91%, 92%, 93%, 94%, 95%, 96%, 97%, 98%, 99% or 100% identical to SEQ ID NO: 1. The human APOE mRNA can encode for a protein comprising a sequence that is about 85%, 87%, 88%, 89%, 90%, 91%, 92%, 93%, 94%, 95%, 96%, 97%, 98%, 99% or 100% identical to SEQ ID NO: 82. The APOE can be an isoform of APOE 2, APOE 3, APOE 4, APOE 5f, APOE 5s, and APOE 7. The hybridization can be by Watson-Crick base pairing.

In certain embodiments, the antisense oligonucleotide can hybridize with human APOE pre-mRNA. The human APOE pre-mRNA can comprise a sequence that is at least 80%, 85%, 87%, 88%, 89%, 90%, 91%, 92%, 93%, 94%, 95%, 96%, 97%, 98%, 99% or 100% identical to SEQ ID NO: 230. The human APOE pre-mRNA can encode for a protein comprising a sequence that is about 85%, 87%, 88%, 89%, 90%, 91%, 92%, 93%, 94%, 95%, 96%, 97%, 98%, 99% or 100% identical to SEQ ID NO: 82. The APOE can be an isoform of APOE 2, APOE 3, APOE 4, APOE 5f, APOE 5s, or APOE 7. The hybridization can be by Watson-Crick base pairing.

The antisense oligonucleotide can hybridize with APOE mRNA or APOE pre-mRNA containing a single-nucleotide polymorphism (SNP). Any single-nucleotide polymorphism can be present in the target APOE mRNA or APOE pre-mRNA (e.g., the mRNA or pre-mRNA ASOs described herein hybridize to), including but not limited to apoE3-R136S (“Christchurch” variant, apoE3-Ch), apoE3-V236E (apoE3-Jacksonville, apoE3-Jac), apoE4-R251G, or combinations of any of the foregoing. In certain embodiments, the APOE mRNA or APOE pre-mRNA contains a single-nucleotide polymorphism (SNP) and the SNP is selected from apoE3-R136S (“Christchurch” variant, apoE3-Ch), apoE3-V236E (apoE3-Jacksonville, apoE3-Jac), apoE4-R251G, or combinations of any of the foregoing.

### Methods of Treatment

This disclosure further relates to methods of using oligonucleotides and pharmaceutically related salts disclosed herein for the treatment of diseases and disorders (e.g., human diseases and disorders, neurodegenerative diseases). The mode of action of an oligonucleotide, such as the ones described herein, differ from the mode of action of an antibody or small molecule, and oligonucleotides are highly advantageous regarding, for example,
(i) the penetration of tissue such as brain tissue which is better than penetration of antibodies due to the substantially smaller size and potentially direct delivery to the brain (e.g., Noguchi Y, et al. (2017) MAbs 9:1210-1215; Pizzo M E, et al. (2018) J Physiol 596:445-475), (ii) prolonged retention and effect duration in the brain after direct administration, (iii) the blocking of multiple functions and activities, respectively, of a target, (iv) the combination of oligonucleotides with each other or an antibody or a small molecule, and (v) the inhibition of intracellular effects which are not accessible for an antibody or not inhibitable via a small molecule.

Taken together for AD or neurodegenerative diseases generally, there is a clear need for effective treatments that can target underlying pathobiological mechanisms. Counteracting the pathogenic gain of toxic function effects of APOE variants could be a therapeutic approach for patients who carry APOE ε4 and have a diagnosis of AD, DLB, PDD (see Li Y, Macyczko J R, Liu C C, Bu G (2022) Neurobiol Aging 115:20-28), or any other human patient or diseases associated with APOE dysregulation. Specifically, the therapeutic effects can be achieved by suppressing the expression of mRNA coding for APOE. The therapeutic effects can also be achieved by suppressing the expression of pre-mRNA coding for APOE.

Two competing technologies are predominantly described for specific suppression of mRNA expression: siRNA and antisense oligonucleotides (ASOs). siRNAs target the mRNA in the cytoplasm. In contrast, RNase H dependent ASOs target the pre-mRNA in the nucleus. Thereby intronic regions can also be targeted by ASOs that are not accessible to siRNA, resulting in a significantly larger target space for ASOs. Moreover, due to its double stranded nature, siRNA does not cross the cell membrane by itself and delivery systems are required for its activity in vitro and in vivo. While delivery systems for siRNA exist that efficiently deliver siRNA to liver cells in vivo, there is currently no system that can deliver siRNA in vivo to extra-hepatic tissues with sufficient efficacy. In contrast, modified ASOs can enter many cell types in vitro without transfection or delivery methods in sufficient amounts, resulting in potent and sequence specific target knockdown. In vivo, naked unconjugated ASOs achieve target knockdown in several different relevant tissues after systemic administration. After direct administration, ASOs achieve a potent and long-lasting target knockdown in the central nervous system (CNS) indicating the strong potential of ASOs for treatment of CNS diseases such as neurodegenerative diseases (see Geary R S et al. (2015) Adv Drug Deliv Rev 87:46-51).

In certain embodiments, the disclosure relates to a method of treating a disease or disorder in a human subject in need thereof comprising administering an antisense oligonucleotide or pharmaceutically acceptable salt thereof as described herein to a subject in need thereof.

In another aspect, the disclosure relates to a method of treating a neurodegenerative disease comprising administering an antisense oligonucleotide or pharmaceutically acceptable salt thereof as described herein to a subject in need thereof. The neurodegenerative disease can be Alzheimer's disease, amyotrophic lateral sclerosis, Huntington's disease, Lewy body dementia, multiple sclerosis, multiple system atrophy, Parkinson's disease, prion disease, or a combination thereof. The neurodegenerative disease can be Alzheimer's disease. The neurodegenerative disease can be a tauopathy. The tauopathy can be primary age-related tauopathy, chronic traumatic encephalopathy, progressive supranuclear palsy, corticobasal degeneration, frontotemporal dementia and parkinsonism linked to chromosome 17, vacuolar tauopathy, lytico-bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, behavioral variant frontotemporal dementia (Pick's disease), argyrophilic grain disease, or a combination thereof.

In another aspect, the disclosure relates to a method of treating an acute or chronic injury comprising administering an antisense oligonucleotide or pharmaceutically acceptable salt thereof as described herein to a subject in need thereof. The acute or chronic injury can be any injury to the nervous system including but not limited to diffuse axonal injuries, concussion, edema, hematoma, skull fractures, hemorrhage, hypoxic brain injury, anoxic brain injury, carbon monoxide poisoning, brain aneurysm, stroke, hydrocephalus, traumatic brain injury, spinal cord injury, chronic traumatic encephalopathy, chemo brain, neuronal damage from tumors, neuronal injury as consequence of meningitis, encephalitis and other inflammation in the body or nervous system or a combination thereof.

In another aspect, the disclosure relates to a method of treating an acute or chronic inflammatory condition in the nervous system or in the body periphery comprising administering an antisense oligonucleotide or pharmaceutically acceptable salt thereof as described herein to a subject in need thereof. The acute or chronic inflammatory condition can be an autoimmune disease, multiple sclerosis, infectious disease, immune response to cancer, a tumor or a combination thereof. The acute or chronic inflammatory condition can be an autoimmune disease, stroke, multiple sclerosis, infectious disease, immune response to cancer, a tumor or a combination thereof.

In another aspect, the disclosure relates to a method of treating a vascular disease or a vascular pathological condition comprising administering an antisense oligonucleotide or pharmaceutically acceptable salt thereof as described herein to a subject in need thereof. The vascular disease or the vascular pathological condition can be any human vascular disease including but limited to cerebrovascular disease, cerebral small vessel disease, diseases caused or influenced by blood-brain barrier leakage, atherosclerosis, cerebral amyloid angiopathy, peripheral vascular disease, peripheral artery disease carotid artery disease, pulmonary embolism, abdominal aortic aneurism, collagen vascular disease, chronic venous insufficiency thrombosis, deep vein thrombosis, or a combination thereof.

In another aspect, the disclosure relates to a method of treating a lipid storage disorder comprising administering an antisense oligonucleotide or pharmaceutically acceptable salt thereof as described herein to a subject in need thereof. The lipid storage disorder can be Cerebrotendineous xanthomatosis, Farber disease, Fabry disease, fucosidosis, Gaucher disease, GM1 gangliosidoses, GM2-gangliosidosis AB variant, Krabbe disease, metachromatic leukodystrophy, multiple sulfatase deficiency, Niemann Pick disease type A, Niemann Pick disease type B, Niemann-Pick disease type C, Sandhoff disease, Schindler disease, Tay-Sachs disease, Wolman disease, or a combination thereof.

In another aspect, the disclosure relates to use of an antisense oligonucleotide or pharmaceutically acceptable salt thereof as described herein for the manufacture of a medicament for the therapeutic treatment of a disease or disorder in a human subject in need thereof.

In another aspect, the disclosure relates to use of an antisense oligonucleotide or pharmaceutically acceptable salt thereof as described herein for treatment of a disease or disorder in a human subject in need thereof.

In another aspect, the disclosure relates to use of an antisense oligonucleotide or pharmaceutically acceptable salt thereof as described herein for the manufacture of a medicament for the therapeutic treatment of a neurodegenerative disease in a subject in need thereof.

In another aspect, the disclosure relates to use of an antisense oligonucleotide or pharmaceutically acceptable salt thereof as described herein for treatment of a neurodegenerative disease in a subject in need thereof. The neurodegenerative disease can be Alzheimer's disease, amyotrophic lateral sclerosis, Huntington's disease, Lewy body dementia, multiple sclerosis, multiple system atrophy, Parkinson's disease, prion disease, or a combination thereof. The neurodegenerative disease can be Alzheimer's disease. The neurodegenerative disease can be a tauopathy. The tauopathy can be primary age-related tauopathy, chronic traumatic encephalopathy, progressive supranuclear palsy, corticobasal degeneration, frontotemporal dementia and parkinsonism linked to chromosome 17, vacuolar tauopathy, lytico-bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, behavioral variant frontotemporal dementia (Pick's disease), argyrophilic grain disease, or a combination thereof.

In certain embodiments, the oligonucleotide or pharmaceutically acceptable salt thereof is administered locally to a subject in need thereof. The local administration can be an intracranial, intracerebral, intramuscular, spinal, epidural, sacroiliac, or a subdural injection.

In certain embodiments, the oligonucleotide or pharmaceutically acceptable salt thereof is administered systemically to a subject in need thereof.

### EXAMPLES

The following examples illustrate non-limiting embodiments of the present invention. The following experiments were performed on cells endogenously expressing APOE, i.e., the cells do not represent an artificial system comprising transfected reporter constructs. Such artificial systems generally show a higher degree of inhibition and lower IC50 values than endogenous systems which are closer to therapeutically relevant in vivo systems.

In certain embodiments this disclosure provides antisense oligonucleotide compounds targeting a human APOE nucleic acid. In certain embodiments the human APOE nucleic acid is the APOE3 variant sequence set forth in NCBI Reference Sequence NM_000041.4.

```
(SEQ ID NO: 1)
      1 ctactcagcc ccagcggagg tgaaggacgt ccttccccag gagccgactg gccaatcaca
    61 ggcaggaaga tgaaggttct gtgggctgcg ttgctggtca cattcctggc aggatgccag
  121 gccaaggtgg agcaagcggt ggagacagag ccggagcccg agctgcgcca gcagaccgag
  181 tggcagagcg gccagcgctg ggaactggca ctgggtcgct tttgggatta cctgcgctgg
  241 gtgcagacac tgtctgagca ggtgcaggag gagctgctca gctcccaggt cacccaggaa
  301 ctgagggcgc tgatggacga gaccatgaag gagttgaagg cctacaaatc ggaactggag
  361 gaacaactga ccccggtggc ggaggagacg cgggcacggc tgtccaagga gctgcaggcg
  421 gcgcaggccc ggctgggcgc ggacatggag gacgtgtgcg gccgcctggt gcagtaccgc
  481 ggcgaggtgc aggccatgct cggccagagc accgaggagc tgcgggtgcg cctcgcctcc
  541 cacctgcgca agctgcgtaa gcggctcctc cgcgatgccg atgacctgca gaagcgcctg
  601 gcagtgtacc aggccggggc ccgcgagggc gccgagcgcg gcctcagcgc catccgcgag
  661 cgcctggggc ccctggtgga acagggccgc gtgcgggccg ccactgtggg ctccctggcc
  721 ggccagccgc tacaggagcg ggcccaggcc tggggcgagc ggctgcgcgc gcggatggag
  781 gagatgggca gccggacccg cgaccgcctg gacgaggtga aggagcaggt ggcggaggtg
  841 cgcgccaagc tggaggagca ggcccagcag atacgcctgc aggccgaggc cttccaggcc
  901 cgcctcaaga gctggttcga gcccctggtg gaagacatgc agcgccagtg ggccgggctg
  961 gtggagaagg tgcaggctgc cgtgggcacc agcgccgccc ctgtgcccag cgacaatcac
1021 tgaacgccga agcctgcagc catgcgaccc cacgccaccc cgtgcctcct gcctccgcgc
1081 agcctgcagc gggagaccct gtccccgccc cagccgtcct cctgggggg accctagttt
1141 aataaagatt caccaagttt cacgca
(SEQ ID NO: 82)
MKVEQAVETEPEPELRQQTEWQSGQRWELALGRFWDYLRWVQTLSEQVQEELLSSQV
TQELRALMDETMKELKAYKSELEEQLTPVAEETRARLSKELQAAQARLGADMEDVCG
RLVQYRGEVQAMLGQSTEELRVRLASHLRKLRKRLLRDADDLQKRLAVYQAGAREGA
ERGLSAIRERLGPLVEQGRVRAATVGSLAGQPLQERAQAWGERLRARMEEMGSRTRD
RLDEVKEQVAEVRAKLEEQAQQIRLQAEAFQARLKSWFEPLVEDMQRQWAGLVEKVQ
AAVGTSAAPVPSDNH
```

In certain embodiments, this disclosure provides antisense oligonucleotide compounds that target a human APOE pre-mRNA. An exemplary DNA sequence of APOE pre-mRNA is:

```
(SEQ ID NO: 230)
      1 CTACTCAGCC CCAGCGGAGG TGAAGGACGT CCTTCCCCAG GAGCCGgtga gaagcgcagt
    61 cgggggcacg gggatgagct caggggcctc tagaaagagc tgggaccctg ggaacccctg
  121 gcctccaggt agtctcagga gagctactcg gggtcgggct tggggagagg aggagcgggg
  181 gtgaggcaag cagcagggga ctggacctgg gaagggctgg gcagcagaga cgacccgacc
  241 cgctagaagg tggggtgggg agagcagctg gactgggatg taagccatag caggactcca
  301 cgagttgtca ctatcattta tcgagcacct actgggtgtc cccagtgtcc tcagatctcc
  361 ataactgggg agccaggggc agcgacacgg tagctagccg tcgattggag aactttaaaa
  421 tgaggactga attagctcat aaatggaaca cggcgcttaa ctgtgaggtt ggagcttaga
  481 atgtgaaggg agaatgagga atgcgagact gggactgaga tggaaccggc ggtggggagg
  541 gggtgggggg atggaatttg aaccccggga gaggaagatg gaattttcta tggaggccga
  601 cctggggatg gggagataag agaagaccag gagggagtta aatagggaat gggttggggg
  661 cggcttggta aatgtgctgg gattaggctg ttgcagataa tgcaacaagg cttggaaggc
  721 taacctgggg tgaggccggg ttggggccgg gctgggggtg ggaggagtcc tcactggcgg
  781 ttgattgaca gtttctcctt ccccagACTG GCCAATCACA GGCAGGAAGA TGAAGGTTCT
  841 GTGGGCTGCG TTGCTGGTCA CATTCCTGGC AGgtatgggg gcggggcttg ctcggttccc
  901 cccgctcctc cccctctcat cctcacctca acctcctggc cccattcagg cagaccctgg
  961 gccccctctt ctgaggcttc tgtgctgctt cctggctctg aacagcgatt tgacgctctc
1021 tgggcctcgg tttcccccat ccttgagata ggagttagaa gttgttttgt tgttgttgtt
1081 tgttgttgtt gttttgtttt tttgagatga agtctcgctc tgtcgcccag gctggagtgc
1141 agtggcggga tctcggctca ctgcaagctc cgcctcccag gtccacgcca ttctcctgcc
1201 tcagcctccc aagtagctgg gactacaggc acatgccacc acacccgact aacttttttg
1261 tattttcagt agagacgggg tttcaccatg ttggccaggc tggtctggaa ctcctgacct
1321 caggtgatct gcccgtttcg atctcccaaa gtgctgggat tacaggcgtg agccaccgca
1381 cctggctggg agttagaggt ttctaatgca ttgcaggcag atagtgaata ccagacacgg
1441 ggcagctgtg atctttattc tccatcaccc ccacacagcc ctgcctgggg cacacaagga
1501 cactcaatac atgcttttcc gctgggcgcg gtggctcacc cctgtaatcc cagcactttg
1561 ggaggccaag gtgggaggat cacttgagcc caggagttca acaccagcct gggcaacata
1621 gtgagaccct gtctctacta aaaatacaaa aattagccag gcatggtgcc acacacctgt
1681 gctctcagct actcaggagg ctgaggcagg aggatcgctt gagcccagaa ggtcaaggtt
1741 gcagtgaacc atgttcaggc cgctgcactc cagcctgggt gacagagcaa gaccctgttt
1801 ataaatacat aatgctttcc aagtgattaa accgactccc ccctcaccct gcccaccatg
1861 gctccaaaga agcatttgtg gagcaccttc tgtgtgcccc taggtactag atgcctggac
1921 ggggtcagaa ggaccctgac ccaccttgaa cttgttccac acagGATGCC AGGCCAAGGT
1981 GGAGCAAGCG GTGGAGACAG AGCCGGAGCC CGAGCTGCGC CAGCAGACCG AGTGGCAGAG
2041 CGGCCAGCGC TGGGAACTGG CACTGGGTCG CTTTTGGGAT TACCTGCGCT GGGTGCAGAC
2101 ACTGTCTGAG CAGGTGCAGG AGGAGCTGCT CAGCTCCCAG GTCACCCAGG AACTGAGgtg
2161 agtgtcccca tcctggccct tgaccctcct ggtgggcggc tatacctccc caggtccagg
2221 tttcattctg cccctgtcgc taagtcttgg ggggcctggg tctctgctgg ttctagcttc
2281 ctcttcccat ttctgactcc tggctttagc tctctggaat tctctctctc agctttgtct
2341 ctctctcttc ccttctgact cagtctctca cactcgtcct ggctctgtct ctgtccttcc
2401 ctagctcttt tatatagaga cagagagatg gggtctcact gtgttgccca ggctggtctt
2461 gaacttctgg gctcaagcga tcctcccgcc tcggcctccc aaagtgctgg gattagaggc
2521 atgagccacc ttgcccggcc tcctagctcc ttcttcgtct ctgcctctgc cctctgcatc
2581 tgctctctgc atctgtctct gtctccttct ctcggcctct gccccgttcc ttctctccct
2641 cttgggtctc tctggctcat ccccatctcg cccgccccat cccagccctt ctccccgcct
2701 cccactgtgc gacaccctcc cgccctctcg gccgcagGGC GCTGATGGAC GAGACCATGA
2761 AGGAGTTGAA GGCCTACAAA TCGGAACTGG AGGAACAACT GACCCCGGTG GCGGAGGAGA
2821 CGCGGGCACG GCTGTCCAAG GAGCTGCAGG CGGCGCAGGC CCGGCTGGGC GCGGACATGG
2881 AGGACGTGTG CGGCCGCCTG GTGCAGTACC GCGGCGAGGT GCAGGCCATG CTCGGCCAGA
2941 GCACCGAGGA GCTGCGGGTG CGCCTCGCCT CCCACCTGCG CAAGCTGCGT AAGCGGCTCC
3001 TCCGCGATGC CGATGACCTG CAGAAGCGCC TGGCAGTGTA CCAGGCCGGG GCCCGCGAGG
3061 GCGCCGAGCG CGGCCTCAGC GCCATCCGCG AGCGCCTGGG GCCCCTGGTG GAACAGGGCC
3121 GCGTGCGGGC CGCCACTGTG GGCTCCCTGG CCGGCCAGCC GCTACAGGAG CGGGCCCAGG
3181 CCTGGGGCGA GCGGCTGCGC GCGCGGATGG AGGAGATGGG CAGCCGGACC CGCGACCGCC
3241 TGGACGAGGT GAAGGAGCAG GTGGCGGAGG TGCGCGCCAA GCTGGAGGAG CAGGCCCAGC
3301 AGATACGCCT GCAGGCCGAG GCCTTCCAGG CCCGCCTCAA GAGCTGGTTC GAGCCCCTGG
3361 TGGAAGACAT GCAGCGCCAG TGGGCCGGGC TGGTGGAGAA GGTGCAGGCT GCCGTGGGCA
3421 CCAGCGCCGC CCCTGTGCCC AGCGACAATC ACTGAACGCC GAAGCCTGCA GCCATGCGAC
3481 CCCACGCCAC CCCGTGCCTC CTGCCTCCGC GCAGCCTGCA GCGGGAGACC CTGTCCCCGC
3541 CCCAGCCGTC CTCCTGGGGT GGACCCTAGT TTAATAAAGA TTCACCAAGT TTCACGCA,
```

wherein capitalization indicates exons and lack thereof indicates introns in the APOE 3 pre-mRNA. This sequence contains a 5′ untranslated region (5′ UTR). The sequence was obtained from the University of California Santa Cruz Genomic Institute Genome Browser under identification number: hg38 ENST00000252486.9.

The present disclosure relates to an oligonucleotide comprising about 18 to 20 nucleotides, at least one nucleotide having a modification selected from the group consisting of a bridged nucleic acid such as LNA, ENA, cET, a 2′-Fluoro modified nucleotide, a 2 O-methyl modified nucleotide, a 2 O-methoxy modified nucleotide, a 2′-fluoroarabino nucleic acid (FANA) and a combination thereof, wherein said modification is located in a sequence of 5 nucleotides of the 5′-end and/or 3′-end of the oligonucleotide and wherein the oligonucleotide hybridizes with a transcript or an mRNA of APOE of SEQ ID NO: 1. The oligonucleotide hybridizes, for example, outside a hybridizing active region or within a hybridizing active region of position 39, 41, 42, 77, 84, 124, 128, 130, 131, 204, −206, 208-214, 226-228, 295-296, 298-299, 307-309, 311, 339, 341-348, 447, 541-542, 550, 573-576, 580, 589, 592, 809, 812, 901-905, 1000-1004, 1006-1014, 1016-1017, 1019-1020, 1122-1123, 1145-1147.

The present disclosure relates to an oligonucleotide comprising about 14 to 18 nucleotides, at least one nucleotide having a modification selected from the group consisting of a bridged nucleic acid (e.g., LNA, ENA, cET, a 2′-Fluoro modified nucleotide, a 2 O-methyl modified nucleotide, a 2 O-methoxy modified nucleotide, a 2′-fluoroarabino nucleic acid (FANA) and a combination thereof), wherein said modification is located in a sequence of 5 nucleotides of the 5′-end and/or 3′-end of the oligonucleotide and wherein the oligonucleotide hybridizes with a transcript or a pre-mRNA of APOE of SEQ ID NO: 230. The oligonucleotide hybridizes, for example, outside a hybridizing active region or within a hybridizing active region of position 45, 90, 127, 268, 269, 272, 273, 275, 276, 297, 298, 301, 319, 343, 344, 346, 347, 354, 380, 381, 384, 390, 391, 392, 394, 399, 400, 440, 444, 445, 446, 447, 448, 450, 451, 452, 453, 454, 456, 459, 462, 463, 464, 465, 485, 488, 489, 490, 492, 586, 769, 770, 788, 915, 916, 917, 919, 966, 967, 968, 969, 1005, 1006, 1007, 1008, 1009, 1036, 1037, 1327, 1328, 1329, 1330, 1331, 1333, 1334, 1335, 1336, 1386, 1424, 1425, 1491, 1725, 1726, 1727, 1728, 1746, 1823, 1855, 1856, 1857, 1875, 1898, 1899, 1900, 1902, 1904, 1905, 1906, 1925, 1950, 1951, 1985, 2148, 2150, 2151, 2211, 2233, 2234, 2235, 2236, 2260, 2261, 2347, 2348, 2355, 2356, 2357, 2358, 2359, 2360, 2362, 2363, 2364, 2699, 2700, 2704, 2743, 2775, 2777, 2778, 2779, 2780, 2781, 3337, 3441, 3442, 3443, 3444, 3445, 3447, 3448, 3449, 3453, 3455, 3582, or 3583.

Modified oligonucleotides complementary to an APOE nucleic acid can be designed and tested for their effect on APOE mRNA in vitro. The modified oligonucleotides can be tested in a series of experiments that had similar culture conditions.

The modified oligonucleotides in the table below can be uniformly modified oligonucleotides. The oligonucleotides can 21 nucleobases in length and each nucleoside can have a 2′-substitution or modification as described herein.

The modified oligonucleotides in Tables 1 and 2 below can be designed as gapmers. The gapmers can be 20 nucleosides in length, wherein the central gap segment comprises 10 2′-deoxynucleosides and is flanked by wing segments on both the 5′ end and on the 3′ end comprising five nucleosides each (5-10-5 gapmers, except ASO S1095, SEQ ID NO: 81, is a 4-10-4 gapmer, Table 1). Each nucleoside in the 5′ wing segment and each nucleoside in the 3′ wing segment comprises a 2′-modification. The 2′-modification is a 2′-methoxyethyl (e.g., 2′-MOE) modification.

In embodiments, each internucleoside linkage is a phosphorothioate internucleoside linkage.

TABLE 1
ASO sequences and corresponding target positions on APOE

| | | | | | | Normalized to non-silencing control | | | |
|---|---|---|---|---|---|---|---|---|---|
| | | | | | | ASO (SEQ ID NO: 2) | | | |
| | | | | | | *Inter-plate average of biological | | | |
| | | | | | | triplicates, normalized knockdown | | | |
| | | | | | | | | | % of fold |
| | | | | | | | | | change in |
| | | Position | | | | | Mean | | HPRT1 |
| | | on | | | | | of | | relative |
| | ASO | tran- | Target | | | | fold | APOE | to non- |
| | SEQ | script | Sequence | | ASO | APOE | change | mRNA | silencing |
| ASO | ID | (Seq ID | SEQ ID | Target | Sequence | mRNA | in | KD | control |
| NO: | NO: | NO: 1) | NO: | Sequence | (5′-3′) | expression* | HPRT1 | (%)* | ASO |
| S1016 | 2 | Non | n/a | n/a | CCTATAGG | 1 | 1 | 0 | 100 |
| | | silencing | | | ACTATCCA | | | | |
| | | control | | | GGAA | | | | |
| S1017 | 3 | 39 | 231 | AGGAGCCG | TGATTGGCC | 0.66 | 0.89 | 33.58 | 11.25 |
| | | | | ACTGGCCA | AGTCGGCT | | | | |
| | | | | ATCA | CCT | | | | |
| S1018 | 4 | 41 | 232 | GAGCCGAC | TGTGATTGG | 0.62 | 0.91 | 38.07 | 8.77 |
| | | | | TGGCCAAT | CCAGTCGG | | | | |
| | | | | CACA | CTC | | | | |
| S1019 | 5 | 42 | 233 | AGCCGACT | CTGTGATTG | 0.47 | 0.86 | 52.63 | 13.85 |
| | | | | GGCCAATC | GCCAGTCG | | | | |
| | | | | ACAG | GCT | | | | |
| S1020 | 6 | 77 | 234 | TTCTGTGG | CAGCAACG | 0.45 | 0.83 | 55.00 | 17.14 |
| | | | | GCTGCGTT | CAGCCCAC | | | | |
| | | | | GCTG | AGAA | | | | |
| S1021 | 7 | 84 | 235 | GGCTGCGT | ATGTGACC | 0.40 | 1.09 | 59.57 | 109.04 |
| | | | | TGCTGGTC | AGCAACGC | | | | |
| | | | | ACAT | AGCC | | | | |
| S1022 | 8 | 124 | 236 | AAGGTGGA | TCCACCGCT | 0.39 | 0.94 | 61.40 | 6.22 |
| | | | | GCAAGCGG | TGCTCCACC | | | | |
| | | | | TGGA | TT | | | | |
| S1023 | 9 | 128 | 237 | TGGAGCAA | TGTCTCCAC | 0.49 | 0.93 | 50.89 | 7.28 |
| | | | | GCGGTGGA | CGCTTGCTC | | | | |
| | | | | GACA | CA | | | | |
| S1024 | 10 | 130 | 238 | GAGCAAGC | TCTGTCTCC | 0.28 | 0.99 | 72.30 | 0.96 |
| | | | | GGTGGAGA | ACCGCTTGC | | | | |
| | | | | CAGA | TC | | | | |
| S1025 | 11 | 131 | 239 | AGCAAGCG | CTCTGTCTC | 0.27 | 0.71 | 73.32 | 29.02 |
| | | | | GTGGAGAC | CACCGCTTG | | | | |
| | | | | AGAG | CT | | | | |
| S1026 | 12 | 204 | 240 | ACTGGCAC | AAAAGCGA | 0.56 | 0.78 | 44.07 | 22.31 |
| | | | | TGGGTCGC | CCCAGTGC | | | | |
| | | | | TTTT | CAGT | | | | |
| S1027 | 13 | 205 | 241 | CTGGCACT | CAAAAGCG | 0.65 | 0.72 | 35.43 | 27.68 |
| | | | | GGGTCGCT | ACCCAGTG | | | | |
| | | | | TTTG | CCAG | | | | |
| S1028 | 14 | 206 | 242 | TGGCACTG | CCAAAAGC | 0.57 | 0.71 | 43.01 | 29.09 |
| | | | | GGTCGCTT | GACCCAGT | | | | |
| | | | | TTGG | GCCA | | | | |
| S1029 | 15 | 208 | 243 | GCACTGGG | TCCCAAAA | 0.38 | 0.94 | 61.98 | 6.07 |
| | | | | TCGCTTTT | GCGACCCA | | | | |
| | | | | GGGA | GTGC | | | | |
| S1030 | 16 | 209 | 244 | CACTGGGT | ATCCCAAA | 0.48 | 0.94 | 52.23 | 6.14 |
| | | | | CGCTTTTG | AGCGACCC | | | | |
| | | | | GGAT | AGTG | | | | |
| S1031 | 17 | 210 | 245 | ACTGGGTC | AATCCCAA | 0.51 | 0.99 | 48.70 | 0.95 |
| | | | | GCTTTTGG | AAGCGACC | | | | |
| | | | | GATT | CAGT | | | | |
| S1032 | 18 | 211 | 246 | CTGGGTCG | TAATCCCA | 0.56 | 0.94 | 44.11 | 5.56 |
| | | | | CTTTTGGG | AAAGCGAC | | | | |
| | | | | ATTA | CCAG | | | | |
| S1033 | 19 | 212 | 247 | TGGGTCGC | GTAATCCC | 0.64 | 0.97 | 36.00 | 2.53 |
| | | | | TTTTGGGA | AAAAGCGA | | | | |
| | | | | TTAC | CCCA | | | | |
| S1034 | 20 | 213 | 248 | GGGTCGCT | GGTAATCC | 0.46 | 0.88 | 53.62 | 12.12 |
| | | | | TTTGGGAT | CAAAAGCG | | | | |
| | | | | TACC | ACCC | | | | |
| S1035 | 21 | 214 | 249 | GGTCGCTT | AGGTAATC | 0.49 | 0.85 | 51.49 | 14.54 |
| | | | | TTGGGATT | CCAAAAGC | | | | |
| | | | | ACCT | GACC | | | | |
| S1036 | 22 | 226 | 250 | GATTACCT | TGCACCCA | 0.46 | 0.44 | 54.08 | 56.09 |
| | | | | GCGCTGGG | GCGCAGGT | | | | |
| | | | | TGCA | AATC | | | | |
| S1037 | 23 | 227 | 251 | ATTACCTG | CTGCACCC | 0.45 | 0.62 | 55.20 | 38.40 |
| | | | | CGCTGGGT | AGCGCAGG | | | | |
| | | | | GCAG | TAAT | | | | |
| S1038 | 24 | 228 | 252 | TTACCTGC | TCTGCACCC | 0.44 | 0.58 | 55.81 | 41.53 |
| | | | | GCTGGGTG | AGCGCAGG | | | | |
| | | | | CAGA | TAA | | | | |
| S1039 | 25 | 295 | 253 | CAGGAACT | ATCAGCGC | 0.38 | 0.76 | 62.41 | 24.25 |
| | | | | GAGGGCGC | CCTCAGTTC | | | | |
| | | | | TGAT | CTG | | | | |
| S1040 | 26 | 296 | 254 | AGGAACTG | CATCAGCG | 0.39 | 0.82 | 60.78 | 18.24 |
| | | | | AGGGCGCT | CCCTCAGTT | | | | |
| | | | | GATG | CCT | | | | |
| S1041 | 27 | 298 | 255 | GAACTGAG | TCCATCAGC | 0.24 | 0.78 | 75.90 | 21.62 |
| | | | | GGCGCTGA | GCCCTCAGT | | | | |
| | | | | TGGA | TC | | | | |
| S1042 | 28 | 299 | 256 | AACTGAGG | GTCCATCA | 0.15 | 0.68 | 84.84 | 32.20 |
| | | | | GCGCTGAT | GCGCCCTC | | | | |
| | | | | GGAC | AGTT | | | | |
| S1043 | 29 | 307 | 257 | GCGCTGAT | ATGGTCTCG | 0.12 | 0.59 | 87.84 | 40.70 |
| | | | | GGACGAG | TCCATCAGC | | | | |
| | | | | ACCAT | GC | | | | |
| S1044 | 30 | 308 | 258 | CGCTGATG | CATGGTCTC | 0.23 | 0.77 | 76.55 | 23.41 |
| | | | | GACGAGAC | GTCCATCA | | | | |
| | | | | CATG | GCG | | | | |
| S1045 | 31 | 309 | 259 | GCTGATGG | TCATGGTCT | 0.27 | 0.88 | 73.30 | 12.45 |
| | | | | ACGAGACC | CGTCCATCA | | | | |
| | | | | ATGA | GC | | | | |
| S1046 | 32 | 311 | 260 | TGATGGAC | CTTCATGGT | 0.34 | 0.95 | 65.84 | 4.76 |
| | | | | GAGACCAT | CTCGTCCAT | | | | |
| | | | | GAAG | CA | | | | |
| S1047 | 33 | 339 | 261 | GGCCTACA | CCAGTTCCG | 0.29 | 1.03 | 71.45 | 102.70 |
| | | | | AATCGGAA | ATTTGTAGG | | | | |
| | | | | CTGG | CC | | | | |
| S1048 | 34 | 341 | 262 | CCTACAAA | CTCCAGTTC | 0.34 | 0.89 | 66.34 | 10.56 |
| | | | | TCGGAACT | CGATTTGTA | | | | |
| | | | | GGAG | GG | | | | |
| S1049 | 35 | 342 | 263 | CTACAAAT | CCTCCAGTT | 0.41 | 0.98 | 58.95 | 2.45 |
| | | | | CGGAACTG | CCGATTTGT | | | | |
| | | | | GAGG | AG | | | | |
| S1050 | 36 | 343 | 264 | TACAAATC | TCCTCCAGT | 0.40 | 1.06 | 59.52 | 106.03 |
| | | | | GGAACTGG | TCCGATTTG | | | | |
| | | | | AGGA | TA | | | | |
| S1051 | 37 | 344 | 265 | ACAAATCG | TTCCTCCAG | 0.40 | 1.21 | 60.32 | 121.26 |
| | | | | GAACTGGA | TTCCGATTT | | | | |
| | | | | GGAA | GT | | | | |
| S1052 | 38 | 345 | 266 | CAAATCGG | GTTCCTCCA | 0.36 | 1.19 | 63.85 | 118.6 |
| | | | | AACTGGAG | GTTCCGATT | | | | 8 |
| | | | | GAAC | TG | | | | |
| S1053 | 39 | 346 | 267 | AAATCGGA | TGTTCCTCC | 0.49 | 1.26 | 51.25 | 125.97 |
| | | | | ACTGGAGG | AGTTCCGAT | | | | |
| | | | | AACA | TT | | | | |
| S1054 | 40 | 347 | 268 | AATCGGAA | TTGTTCCTC | 0.50 | 1.12 | 50.40 | 111.90 |
| | | | | CTGGAGGA | CAGTTCCG | | | | |
| | | | | ACAA | ATT | | | | |
| S1055 | 41 | 348 | 269 | ATCGGAAC | GTTGTTCCT | 0.41 | 1.13 | 58.60 | 112.85 |
| | | | | TGGAGGAA | CCAGTTCCG | | | | |
| | | | | CAAC | AT | | | | |
| S1056 | 42 | 541 | 270 | CACCTGCG | TTACGCAG | 0.53 | 1.14 | 46.61 | 114.49 |
| | | | | CAAGCTGC | CTTGCGCA | | | | |
| | | | | GTAA | GGTG | | | | |
| S1057 | 43 | 542 | 271 | ACCTGCGC | CTTACGCA | 0.48 | 1.10 | 52.45 | 109.50 |
| | | | | AAGCTGCG | GCTTGCGC | | | | |
| | | | | TAAG | AGGT | | | | |
| S1058 | 44 | 550 | 272 | AAGCTGCG | AGGAGCCG | 0.55 | 1.10 | 45.08 | 110.29 |
| | | | | TAAGCGGC | CTTACGCA | | | | |
| | | | | TCCT | GCTT | | | | |
| S1059 | 45 | 573 | 273 | CGATGCCG | TCTGCAGGT | 0.30 | 0.94 | 69.67 | 5.98 |
| | | | | ATGACCTG | CATCGGCA | | | | |
| | | | | CAGA | TCG | | | | |
| S1060 | 46 | 574 | 274 | GATGCCGA | TTCTGCAGG | 0.56 | 0.92 | 43.93 | 8.01 |
| | | | | TGACCTGC | TCATCGGC | | | | |
| | | | | AGAA | ATC | | | | |
| S1061 | 47 | 575 | 275 | ATGCCGAT | CTTCTGCAG | 0.49 | 0.96 | 51.05 | 3.59 |
| | | | | GACCTGCA | GTCATCGG | | | | |
| | | | | GAAG | CAT | | | | |
| S1062 | 48 | 576 | 276 | TGCCGATG | GCTTCTGCA | 0.38 | 0.84 | 62.17 | 16.08 |
| | | | | ACCTGCAG | GGTCATCG | | | | |
| | | | | AAGC | GCA | | | | |
| S1063 | 49 | 580 | 277 | GATGACCT | AGGCGCTT | 0.44 | 1.10 | 56.04 | 110.46 |
| | | | | GCAGAAGC | CTGCAGGT | | | | |
| | | | | GCCT | CATC | | | | |
| S1064 | 50 | 589 | 278 | CAGAAGCG | TACACTGCC | 0.49 | 0.74 | 51.26 | 25.70 |
| | | | | CCTGGCAG | AGGCGCTT | | | | |
| | | | | TGTA | CTG | | | | |
| S1065 | 51 | 592 | 279 | AAGCGCCT | TGGTACACT | 0.61 | 0.65 | 39.19 | 35.12 |
| | | | | GGCAGTGT | GCCAGGCG | | | | |
| | | | | ACCA | CTT | | | | |
| S1066 | 52 | 809 | 280 | TGGACGAG | CTGCTCCTT | 0.55 | 0.98 | 45.27 | 2.10 |
| | | | | GTGAAGGA | CACCTCGTC | | | | |
| | | | | GCAG | CA | | | | |
| S1067 | 53 | 812 | 281 | ACGAGGTG | CACCTGCTC | 0.50 | 0.82 | 50.46 | 17.62 |
| | | | | AAGGAGC | CTTCACCTC | | | | |
| | | | | AGGTG | GT | | | | |
| S1068 | 54 | 901 | 282 | CGCCTCAA | TCGAACCA | 0.51 | 1.00 | 48.95 | 100.38 |
| | | | | GAGCTGGT | GCTCTTGAG | | | | |
| | | | | TCGA | GCG | | | | |
| S1069 | 55 | 902 | 283 | GCCTCAAG | CTCGAACC | 0.48 | 0.88 | 52.48 | 12.41 |
| | | | | AGCTGGTT | AGCTCTTGA | | | | |
| | | | | CGAG | GGC | | | | |
| S1070 | 56 | 903 | 284 | CCTCAAGA | GCTCGAAC | 0.44 | 1.07 | 55.94 | 106.57 |
| | | | | GCTGGTTC | CAGCTCTTG | | | | |
| | | | | GAGC | AGG | | | | |
| S1071 | 57 | 904 | 285 | CTCAAGAG | GGCTCGAA | 0.43 | 1.08 | 56.70 | 108.01 |
| | | | | CTGGTTCG | CCAGCTCTT | | | | |
| | | | | AGCC | GAG | | | | |
| S1072 | 58 | 905 | 286 | TCAAGAGC | GGGCTCGA | 0.53 | 0.98 | 47.00 | 1.59 |
| | | | | TGGTTCGA | ACCAGCTCT | | | | |
| | | | | GCCC | TGA | | | | |
| S1073 | 59 | 1000 | 287 | CCTGTGCC | TGATTGTCG | 0.52 | 1.06 | 48.04 | 105.96 |
| | | | | CAGCGACA | CTGGGCAC | | | | |
| | | | | ATCA | AGG | | | | |
| S1074 | 60 | 1001 | 288 | CTGTGCCC | GTGATTGTC | 0.46 | 1.06 | 53.82 | 105.75 |
| | | | | AGCGACAA | GCTGGGCA | | | | |
| | | | | TCAC | CAG | | | | |
| S1075 | 61 | 1002 | 289 | TGTGCCCA | AGTGATTGT | 0.31 | 1.02 | 69.48 | 101.52 |
| | | | | GCGACAAT | CGCTGGGC | | | | |
| | | | | CACT | ACA | | | | |
| S1076 | 62 | 1004 | 290 | TGCCCAGC | TCAGTGATT | 0.34 | 0.92 | 66.14 | 7.53 |
| | | | | GACAATCA | GTCGCTGG | | | | |
| | | | | CTGA | GCA | | | | |
| S1077 | 63 | 1006 | 291 | CCCAGCGA | GTTCAGTG | 0.29 | 1.07 | 71.16 | 107.49 |
| | | | | CAATCACT | ATTGTCGCT | | | | |
| | | | | GAAC | GGG | | | | |
| S1078 | 64 | 1007 | 292 | CCAGCGAC | CGTTCAGTG | 0.30 | 1.02 | 70.43 | 101.68 |
| | | | | AATCACTG | ATTGTCGCT | | | | |
| | | | | AACG | GG | | | | |
| S1079 | 65 | 1008 | 293 | CAGCGACA | GCGTTCAGT | 0.35 | 1.00 | 64.83 | 100.14 |
| | | | | ATCACTGA | GATTGTCGC | | | | |
| | | | | ACGC | TG | | | | |
| S1080 | 66 | 1009 | 294 | AGCGACAA | GGCGTTCA | 0.37 | 1.14 | 63.35 | 113.80 |
| | | | | TCACTGAA | GTGATTGTC | | | | |
| | | | | CGCC | GCT | | | | |
| S1081 | 67 | 1010 | 295 | GCGACAAT | CGGCGTTC | 0.37 | 0.98 | 63.19 | 2.46 |
| | | | | CACTGAAC | AGTGATTGT | | | | |
| | | | | GCCG | CGC | | | | |
| S1082 | 68 | 1011 | 296 | CGACAATC | TCGGCGTTC | 0.41 | 1.01 | 59.16 | 101.20 |
| | | | | ACTGAACG | AGTGATTGT | | | | |
| | | | | CCGA | CG | | | | |
| S1083 | 69 | 1012 | 297 | GACAATCA | TTCGGCGTT | 0.50 | 1.08 | 49.91 | 107.72 |
| | | | | CTGAACGC | CAGTGATT | | | | |
| | | | | CGAA | GTC | | | | |
| S1084 | 70 | 1013 | 298 | ACAATCAC | CTTCGGCGT | 0.50 | 0.97 | 49.54 | 3.04 |
| | | | | TGAACGCC | TCAGTGATT | | | | |
| | | | | GAAG | GT | | | | |
| S1085 | 71 | 1014 | 299 | CAATCACT | GCTTCGGC | 0.42 | 0.92 | 58.13 | 7.8 |
| | | | | GAACGCCG | GTTCAGTG | | | | |
| | | | | AAGC | ATTG | | | | |
| S1086 | 72 | 1016 | 300 | ATCACTGA | AGGCTTCG | 0.38 | 0.95 | 61.55 | 5.28 |
| | | | | ACGCCGAA | GCGTTCAGT | | | | |
| | | | | GCCT | GAT | | | | |
| S1087 | 73 | 1017 | 301 | TCACTGAA | CAGGCTTC | 0.43 | 1.01 | 57.45 | 101.41 |
| | | | | CGCCGAAG | GGCGTTCA | | | | |
| | | | | CCTG | GTGA | | | | |
| S1088 | 74 | 1019 | 302 | ACTGAACG | TGCAGGCTT | 0.28 | 0.91 | 72.20 | 9.36 |
| | | | | CCGAAGCC | CGGCGTTC | | | | |
| | | | | TGCA | AGT | | | | |
| S1089 | 75 | 1120 | 303 | TCCTGGGG | AACTAGGG | 0.36 | 0.79 | 64.14 | 21.32 |
| | | | | TGGACCCT | TCCACCCCA | | | | |
| | | | | AGTT | GGA | | | | |
| S1090 | 76 | 1122 | 304 | CTGGGGTG | TAAACTAG | 0.52 | 0.90 | 47.71 | 10.19 |
| | | | | GACCCTAG | GGTCCACC | | | | |
| | | | | TTTA | CCAG | | | | |
| S1019 | 77 | 1123 | 305 | TGGGGTGG | TTAAACTA | 0.44 | 1.05 | 55.72 | 105.32 |
| | | | | ACCCTAGT | GGGTCCAC | | | | |
| | | | | TTAA | CCCA | | | | |
| S1092 | 78 | 1145 | 306 | AAGATTCA | CGTGAAAC | 0.42 | 1.06 | 58.16 | 106.01 |
| | | | | CCAAGTTT | TTGGTGAAT | | | | |
| | | | | CACG | CTT | | | | |
| S1093 | 79 | 1146 | 307 | AGATTCAC | GCGTGAAA | 0.39 | 1.14 | 61.03 | 113.54 |
| | | | | CAAGTTTC | CTTGGTGA | | | | |
| | | | | ACGC | ATCT | | | | |
| S1094 | 80 | 1147 | 308 | GATTCACC | TGCGTGAA | 0.37 | 1.13 | 62.57 | 112.63 |
| | | | | AAGTTTCA | ACTTGGTG | | | | |
| | | | | CGCA | AATC | | | | |
| S1095 | 81 | 447 | 309 | GGAGGAC | CGGCCGCG | 0.41 | 1.15 | 59.49 | 114.87 |
| | | | | GTGCGCGG | CACGTCCTC | | | | |
| | | | | CCG | C | | | | |

TABLE 2
Selected ASOs

| ASO NO: | SEQ ID NO: |
|---|---|
| S1021 | 7 |
| S1022 | 8 |
| S1024 | 10 |
| S1029 | 15 |
| S1046 | 32 |
| S1047 | 33 |
| S1049 | 35 |
| S1050 | 36 |
| S1051 | 37 |
| S1052 | 38 |
| S1053 | 39 |
| S1054 | 40 |
| S1055 | 41 |
| S1057 | 43 |
| S1059 | 45 |
| S1063 | 49 |
| S1070 | 56 |
| S1071 | 57 |
| S1074 | 60 |
| S1075 | 61 |
| S1076 | 62 |
| S1077 | 63 |
| S1078 | 64 |
| S1079 | 65 |
| S1080 | 66 |
| S1081 | 67 |
| S1082 | 68 |
| S1083 | 69 |
| S1085 | 71 |
| S1086 | 72 |
| S1087 | 73 |
| S1088 | 74 |
| S1091 | 77 |
| S1092 | 78 |
| S1093 | 79 |
| S1094 | 80 |
| S1095 | 81 |

The modified oligonucleotides in Tables 3 and 4 below can be uniformly modified oligonucleotides. The oligonucleotides can be 16 nucleobases in length and each nucleoside can have a 2′-substitution or -modification as described herein.

The modified oligonucleotides in Tables 3 and 4 below can be designed as gapmers. The gapmers can be 16 nucleosides in length, wherein the central gap segment comprises 10 2′-deoxynucleosides and is flanked by wing segments on both the 5′ end and on the 3′ end comprising three nucleosides each (3-10-3 gapmers). Each nucleoside in the 5′ wing segment and each nucleoside in the 3′ wing segment comprises a locked nucleic acid (LNA) modification.

In embodiments, each internucleoside linkage is a phosphorothioate internucleoside linkage.

TABLE 3
ASO sequences and corresponding target positions on APOE pre-mRNA

| | | Position | | | | Normalized to non-silencing | |
|---|---|---|---|---|---|---|---|
| | ASO | on tran- | Target | | | control ASO (SEQ ID NO: 83) | |
| | SEQ | script | Sequence | | ASO | Average of biological triplicates | |
| ASO | ID | (Seq ID | SEQ ID | Target | Sequence | Fold change | Fold change in |
| NO: | NO: | NO:230) | NO: | Sequence | (5′-3′) | in APOE | HPRT1 |
| S1250 | 83 | | n/a | n/a | GGCTACTA | 1.00 | 1.00 |
| | | | | | CGCCGTCA | | |
| S1104 | 84 | 45 | 310 | CGGTGAGA | ACTGCGCT | 0.49 | 0.55 |
| | | | | AGCGCAGT | TCTCACCG | | |
| S1105 | 85 | 90 | 311 | CTAGAAAG | TCCCAGCT | 0.51 | 0.55 |
| | | | | AGCTGGGA | CTTTCTAG | | |
| S1106 | 86 | 127 | 312 | AGGTAGTC | TCTCCTGA | 0.98 | 0.84 |
| | | | | TCAGGAGA | GACTACCT | | |
| S1107 | 87 | 268 | 313 | CTGGACTG | TTACATCC | 0.82 | 0.70 |
| | | | | GGATGTAA | CAGTCCAG | | |
| S1108 | 88 | 269 | 314 | TGGACTGG | CTTACATC | 0.69 | 0.71 |
| | | | | GATGTAAG | CCAGTCCA | | |
| S1109 | 89 | 272 | 315 | ACTGGGAT | TGGCTTAC | 0.71 | 0.60 |
| | | | | GTAAGCCA | ATCCCAGT | | |
| S1110 | 90 | 273 | 316 | CTGGGATG | ATGGCTTA | 0.64 | 0.67 |
| | | | | TAAGCCAT | CATCCCAG | | |
| S1111 | 91 | 275 | 317 | GGGATGTA | CTATGGCT | 0.60 | 0.70 |
| | | | | AGCCATAG | TACATCCC | | |
| S1112 | 92 | 276 | 318 | GGATGTAA | GCTATGGC | 0.81 | 0.65 |
| | | | | GCCATAGC | TTACATCC | | |
| S1113 | 93 | 297 | 319 | TCCACGAG | AGTGACAA | 0.82 | 0.70 |
| | | | | TTGTCACT | CTCGTGGA | | |
| S1114 | 94 | 298 | 320 | CCACGAGT | TAGTGACA | 0.57 | 0.56 |
| | | | | TGTCACTA | ACTCGTGG | | |
| S1115 | 95 | 301 | 321 | CGAGTTGT | TGATAGTG | 0.71 | 0.70 |
| | | | | CACTATCA | ACAACTCG | | |
| S1116 | 96 | 319 | 322 | TATCGAGC | CAGTAGGT | 0.60 | 0.56 |
| | | | | ACCTACTG | GCTCGATA | | |
| S1117 | 97 | 343 | 323 | CAGTGTCC | AGATCTGA | 0.79 | 0.75 |
| | | | | TCAGATCT | GGACACTG | | |
| S1118 | 98 | 344 | 324 | AGTGTCCT | GAGATCTG | 0.77 | 0.60 |
| | | | | CAGATCTC | AGGACACT | | |
| S1119 | 99 | 346 | 325 | TGTCCTCA | TGGAGATC | 0.82 | 0.65 |
| | | | | GATCTCCA | TGAGGACA | | |
| S1120 | 100 | 347 | 326 | GTCCTCAG | ATGGAGAT | 0.70 | 0.58 |
| | | | | ATCTCCAT | CTGAGGAC | | |
| S1121 | 101 | 354 | 327 | GATCTCCA | CCCAGTTA | 0.75 | 0.62 |
| | | | | TAACTGGG | TGGAGATC | | |
| S1122 | 102 | 380 | 328 | CAGCGACA | AGCTACCG | 0.70 | 0.67 |
| | | | | CGGTAGCT | TGTCGCTG | | |
| S1123 | 103 | 381 | 329 | AGCGACAC | TAGCTACC | 0.64 | 0.54 |
| | | | | GGTAGCTA | GTGTCGCT | | |
| S1124 | 104 | 384 | 330 | GACACGGT | GGCTAGCT | 0.67 | 0.48 |
| | | | | AGCTAGCC | ACCGTGTC | | |
| S1125 | 105 | 390 | 331 | GTAGCTAG | ATCGACGG | 0.71 | 0.53 |
| | | | | CCGTCGAT | CTAGCTAC | | |
| S1126 | 106 | 391 | 332 | TAGCTAGC | AATCGACG | 0.86 | 0.80 |
| | | | | CGTCGATT | GCTAGCTA | | |
| S1127 | 107 | 392 | 333 | AGCTAGCC | CAATCGAC | 0.92 | 0.77 |
| | | | | GTCGATTG | GGCTAGCT | | |
| S1128 | 108 | 394 | 334 | CTAGCCGT | TCCAATCG | 0.53 | 0.67 |
| | | | | CGATTGGA | ACGGCTAG | | |
| S1129 | 109 | 399 | 335 | CGTCGATT | AGTTCTCC | 0.72 | 0.80 |
| | | | | GGAGAACT | AATCGACG | | |
| S1130 | 110 | 400 | 336 | GTCGATTG | AAGTTCTC | 0.81 | 0.71 |
| | | | | GAGAACTT | CAATCGAC | | |
| S1131 | 111 | 440 | 337 | TAAATGGA | CGCCGTGT | 0.53 | 0.53 |
| | | | | ACACGGCG | TCCATTTA | | |
| S1132 | 112 | 444 | 338 | TGGAACAC | TAAGCGCC | 0.73 | 0.77 |
| | | | | GGCGCTTA | GTGTTCCA | | |
| S1133 | 113 | 445 | 339 | GGAACACG | TTAAGCGC | 0.56 | 0.56 |
| | | | | GCGCTTAA | CGTGTTCC | | |
| S1134 | 114 | 446 | 340 | GAACACGG | GTTAAGCG | 0.6 | 0.55 |
| | | | | CGCTTAAC | CCGTGTTC | | |
| S1135 | 115 | 447 | 341 | AACACGGC | AGTTAAGC | 0.66 | 0.62 |
| | | | | GCTTAACT | GCCGTGTT | | |
| S1136 | 116 | 448 | 342 | ACACGGCG | CAGTTAAG | 0.72 | 0.61 |
| | | | | CTTAACTG | CGCCGTGT | | |
| S1137 | 117 | 450 | 343 | ACGGCGCT | CACAGTTA | 0.92 | 0.92 |
| | | | | TAACTGTG | AGCGCCGT | | |
| S1138 | 118 | 451 | 344 | CGGCGCTT | TCACAGTT | 0.78 | 0.71 |
| | | | | AACTGTGA | AAGCGCCG | | |
| S1139 | 119 | 452 | 345 | GGCGCTTA | CTCACAGT | 0.48 | 0.45 |
| | | | | ACTGTGAG | TAAGCGCC | | |
| S1140 | 120 | 453 | 346 | GCGCTTAA | CCTCACAG | 0.49 | 0.44 |
| | | | | CTGTGAGG | TTAAGCGC | | |
| S1141 | 121 | 454 | 347 | CGCTTAAC | ACCTCACA | 0.75 | 0.64 |
| | | | | TGTGAGGT | GTTAAGCG | | |
| S1142 | 122 | 456 | 348 | CTTAACTG | CAACCTCA | 0.97 | 0.88 |
| | | | | TGAGGTTG | CAGTTAAG | | |
| S1143 | 123 | 459 | 349 | AACTGTGA | CTCCAACC | 0.39 | 0.47 |
| | | | | GGTTGGAG | TCACAGTT | | |
| S1144 | 124 | 462 | 350 | TGTGAGGT | AAGCTCCA | 0.30 | 0.36 |
| | | | | TGGAGCTT | ACCTCACA | | |
| S1145 | 125 | 463 | 351 | GTGAGGTT | TAAGCTCC | 0.85 | 0.77 |
| | | | | GGAGCTTA | AACCTCAC | | |
| S1146 | 126 | 464 | 352 | TGAGGTTG | CTAAGCTC | 0.81 | 0.89 |
| | | | | GAGCTTAG | CAACCTCA | | |
| S1147 | 127 | 465 | 353 | GAGGTTGG | TCTAAGCT | 1.03 | 1.22 |
| | | | | AGCTTAGA | CCAACCTC | | |
| S1148 | 128 | 485 | 354 | GAAGGGAG | TCCTCATT | 0.91 | 0.88 |
| | | | | AATGAGGA | CTCCCTTC | | |
| S1149 | 129 | 488 | 355 | GGGAGAAT | CATTCCTC | 0.44 | 0.41 |
| | | | | GAGGAATG | ATTCTCCC | | |
| S1150 | 130 | 489 | 356 | GGAGAATG | GCATTCCT | 0.65 | 0.57 |
| | | | | AGGAATGC | CATTCTCC | | |
| S1151 | 131 | 490 | 357 | GAGAATGA | CGCATTCC | 0.52 | 0.46 |
| | | | | GGAATGCG | TCATTCTC | | |
| S1152 | 132 | 492 | 358 | GAATGAGG | CTCGCATT | 0.73 | 0.95 |
| | | | | AATGCGAG | CCTCATTC | | |
| S1153 | 133 | 586 | 359 | TTCTATGG | GTCGGCCT | 0.38 | 0.30 |
| | | | | AGGCCGAC | CCATAGAA | | |
| S1154 | 134 | 769 | 360 | CCTCACTG | TCAACCGC | 0.45 | 0.40 |
| | | | | GCGGTTGA | CAGTGAGG | | |
| S1155 | 135 | 770 | 361 | CTCACTGG | ATCAACCG | 0.48 | 0.42 |
| | | | | CGGTTGAT | CCAGTGAG | | |
| S1156 | 136 | 788 | 362 | ACAGTTTC | GGGAAGGA | 0.49 | 0.40 |
| | | | | TCCTTCCC | GAAACTGT | | |
| S1157 | 137 | 915 | 363 | TCTCATCC | TGAGGTGA | 0.74 | 0.58 |
| | | | | TCACCTCA | GGATGAGA | | |
| S1158 | 138 | 916 | 364 | CTCATCCT | TTGAGGTG | 0.60 | 0.58 |
| | | | | CACCTCAA | AGGATGAG | | |
| S1159 | 139 | 917 | 365 | TCATCCTC | GTTGAGGT | 0.76 | 0.53 |
| | | | | ACCTCAAC | GAGGATGA | | |
| S1160 | 140 | 919 | 366 | ATCCTCAC | AGGTTGAG | 0.74 | 0.57 |
| | | | | CTCAACCT | GTGAGGAT | | |
| S1161 | 141 | 966 | 367 | CTCTTCTG | AGAAGCCT | 0.71 | 0.71 |
| | | | | AGGCTTCT | CAGAAGAG | | |
| S1162 | 142 | 967 | 368 | TCTTCTGA | CAGAAGCC | 0.94 | 0.84 |
| | | | | GGCTTCTG | TCAGAAGA | | |
| S1163 | 143 | 968 | 369 | CTTCTGAG | ACAGAAGC | 0.85 | 0.73 |
| | | | | GCTTCTGT | CTCAGAAG | | |
| S1164 | 144 | 969 | 370 | TTCTGAGG | CACAGAAG | 0.73 | 0.71 |
| | | | | CTTCTGTG | CCTCAGAA | | |
| S1165 | 145 | 1005 | 371 | GCGATTTG | GAGAGCGT | 0.82 | 0.80 |
| | | | | ACGCTCTC | CAAATCGC | | |
| S1166 | 146 | 1006 | 372 | CGATTTGA | AGAGAGCG | 0.86 | 0.84 |
| | | | | CGCTCTCT | TCAAATCG | | |
| S1167 | 147 | 1007 | 373 | GATTTGAC | CAGAGAGC | 0.74 | 0.86 |
| | | | | GCTCTCTG | GTCAAATC | | |
| S1168 | 148 | 1008 | 374 | ATTTGACG | CCAGAGAG | 0.96 | 1.02 |
| | | | | CTCTCTGG | CGTCAAAT | | |
| S1169 | 149 | 1009 | 375 | TTTGACGC | CCCAGAGA | 0.91 | 0.97 |
| | | | | TCTCTGGG | GCGTCAAA | | |
| S1170 | 150 | 1036 | 376 | CCCATCCT | CTATCTCA | 0.64 | 0.58 |
| | | | | TGAGATAG | AGGATGGG | | |
| S1171 | 151 | 1037 | 377 | CCATCCTT | CCTATCTC | 0.70 | 0.66 |
| | | | | GAGATAGG | AAGGATGG | | |
| S1172 | 152 | 1327 | 378 | ATCTGCCC | ATCGAAAC | 1.06 | 0.96 |
| | | | | GTTTCGAT | GGGCAGAT | | |
| S1173 | 153 | 1328 | 379 | TCTGCCCG | GATCGAAA | 0.88 | 0.75 |
| | | | | TTTCGATC | CGGGCAGA | | |
| S1174 | 154 | 1329 | 380 | CTGCCCGT | AGATCGAA | 1.01 | 0.89 |
| | | | | TTCGATCT | ACGGGCAG | | |
| S1175 | 155 | 1330 | 381 | TGCCCGTT | GAGATCGA | 1.21 | 1.14 |
| | | | | TCGATCTC | AACGGGCA | | |
| S1176 | 156 | 1331 | 382 | GCCCGTTT | GGAGATCG | 0.72 | 0.52 |
| | | | | CGATCTCC | AAACGGGC | | |
| S1177 | 157 | 1333 | 383 | CCGTTTCG | TGGGAGAT | 0.58 | 0.52 |
| | | | | ATCTCCCA | CGAAACGG | | |
| S1178 | 158 | 1334 | 384 | CGTTTCGA | TTGGGAGA | 0.96 | 0.73 |
| | | | | TCTCCCAA | TCGAAACG | | |
| S1179 | 159 | 1335 | 385 | GTTTCGAT | TTTGGGAG | 0.75 | 0.69 |
| | | | | CTCCCAAA | ATCGAAAC | | |
| S1180 | 160 | 1336 | 386 | TTTCGATC | CTTTGGGA | 0.87 | 0.86 |
| | | | | TCCCAAAG | GATCGAAA | | |
| S1181 | 161 | 1386 | 387 | CTGGGAGT | AACCTCTA | 0.89 | 0.97 |
| | | | | TAGAGGTT | ACTCCCAG | | |
| S1182 | 162 | 1424 | 388 | GTGAATAC | CGTGTCTG | 0.74 | 0.66 |
| | | | | CAGACACG | GTATTCAC | | |
| S1183 | 163 | 1425 | 389 | TGAATACC | CCGTGTCT | 0.60 | 0.49 |
| | | | | AGACACGG | GGTATTCA | | |
| S1184 | 164 | 1491 | 390 | CACACAAG | TGAGTGTC | 0.69 | 0.72 |
| | | | | GACACTCA | CTTGTGTG | | |
| S1185 | 165 | 1725 | 391 | CCAGAAGG | AACCTTGA | 0.80 | 0.71 |
| | | | | TCAAGGTT | CCTTCTGG | | |
| S1186 | 166 | 1726 | 392 | CAGAAGGT | CAACCTTG | 1.00 | 1.04 |
| | | | | CAAGGTTG | ACCTTCTG | | |
| S1187 | 167 | 1727 | 393 | AGAAGGTC | GCAACCTT | 0.87 | 0.79 |
| | | | | AAGGTTGC | GACCTTCT | | |
| S1188 | 168 | 1728 | 394 | GAAGGTCA | TGCAACCT | 0.44 | 0.37 |
| | | | | AGGTTGCA | TGACCTTC | | |
| S1189 | 169 | 1746 | 395 | GAACCATG | GGCCTGAA | 0.74 | 0.61 |
| | | | | TTCAGGCC | CATGGTTC | | |
| S1190 | 170 | 1823 | 396 | GTGATTAA | GAGTCGGT | 0.65 | 0.6″ |
| | | | | ACCGACTC | TTAATCAC | | |
| S1191 | 171 | 1855 | 397 | ACCATGGC | TCTTTGGA | 0.76 | 0.70 |
| | | | | TCCAAAGA | GCCATGGT | | |
| S1192 | 172 | 1856 | 398 | CCATGGCT | TTCTTTGG | 0.78 | 0.75 |
| | | | | CCAAAGAA | AGCCATGG | | |
| S1193 | 173 | 1857 | 399 | CATGGCTC | CTTCTTTG | 1.05 | 1.02 |
| | | | | CAAAGAAG | GAGCCATG | | |
| S1194 | 174 | 1875 | 400 | TTTGTGGA | GAAGGTGC | 0.72 | 0.66 |
| | | | | GCACCTTC | TCCACAAA | | |
| S1195 | 175 | 1898 | 401 | CCCTAGGT | CATCTAGT | 0.64 | 0.50 |
| | | | | ACTAGATG | ACCTAGGG | | |
| S1196 | 176 | 1899 | 402 | CCTAGGTA | GCATCTAG | 0.63 | 0.58 |
| | | | | CTAGATGC | TACCTAGG | | |
| S1197 | 177 | 1900 | 403 | CTAGGTAC | GGCATCTA | 0.93 | 0.85 |
| | | | | TAGATGCC | GTACCTAG | | |
| S1198 | 178 | 1902 | 404 | AGGTACTA | CAGGCATC | 1.07 | 1.11 |
| | | | | GATGCCTG | TAGTACCT | | |
| S1199 | 179 | 1904 | 405 | GTACTAGA | TCCAGGCA | 0.87 | 0.8 |
| | | | | TGCCTGGA | TCTAGTAC | | |
| S1200 | 180 | 1905 | 406 | TACTAGAT | GTCCAGGC | 0.64 | 0.75 |
| | | | | GCCTGGAC | ATCTAGTA | | |
| S1201 | 181 | 1906 | 407 | ACTAGATG | CGTCCAGG | 0.81 | 0.70 |
| | | | | CCTGGACG | CATCTAGT | | |
| S1202 | 182 | 1925 | 408 | TCAGAAGG | GTCAGGGT | 0.97 | 0.92 |
| | | | | ACCCTGAC | CCTTCTGA | | |
| S1203 | 183 | 1950 | 409 | ACTTGTTC | CCTGTGTG | 0.88 | 0.90 |
| | | | | CACACAGG | GAACAAGT | | |
| S1204 | 184 | 1951 | 410 | CTTGTTCC | TCCTGTGT | 1.00 | 0.98 |
| | | | | ACACAGGA | GGAACAAG | | |
| S1205 | 185 | 1985 | 411 | CAAGCGGT | CTGTCTCC | 0.41 | 0.47 |
| | | | | GGAGACAG | ACCGCTTG | | |
| S1206 | 186 | 2148 | 412 | AGGAACTG | ACTCACCT | 0.75 | 0.72 |
| | | | | AGGTGAGT | CAGTTCCT | | |
| S1207 | 187 | 2150 | 413 | GAACTGAG | ACACTCAC | 0.77 | 0.61 |
| | | | | GTGAGTGT | CTCAGTTC | | |
| S1208 | 188 | 2151 | 414 | AACTGAGG | GACACTCA | 1.00 | 0.96 |
| | | | | TGAGTGTC | CCTCAGTT | | |
| S1209 | 189 | 2211 | 415 | CAGGTCCA | ATGAAACC | 0.82 | 0.82 |
| | | | | GGTTTCAT | TGGACCTG | | |
| S1210 | 190 | 2233 | 416 | CCTGTCGC | AAGACTTA | 1.03 | 0.9 |
| | | | | TAAGTCTT | GCGACAGG | | |
| S1211 | 191 | 2234 | 417 | CTGTCGCT | CAAGACTT | 0.73 | 0.79 |
| | | | | AAGTCTTG | AGCGACAG | | |
| S1212 | 192 | 2235 | 418 | TGTCGCTA | CCAAGACT | 0.79 | 0.71 |
| | | | | AGTCTTGG | TAGCGACA | | |
| S1213 | 193 | 2236 | 419 | GTCGCTAA | CCCAAGAC | 0.71 | 0.57 |
| | | | | GTCTTGGG | TTAGCGAC | | |
| S1214 | 194 | 2260 | 420 | GTCTCTGC | TAGAACCA | 0.88 | 0.87 |
| | | | | TGGTTCTA | GCAGAGAC | | |
| S1215 | 195 | 2261 | 421 | TCTCTGCT | CTAGAACC | 0.93 | 0.81 |
| | | | | GGTTCTAG | AGCAGAGA | | |
| S1216 | 196 | 2347 | 422 | CTTCCCTT | TGAGTCAG | 0.78 | 0.82 |
| | | | | CTGACTCA | AAGGGAAG | | |
| S1217 | 197 | 2348 | 423 | TTCCCTTC | CTGAGTCA | 0.88 | 0.90 |
| | | | | TGACTCAG | GAAGGGAA | | |
| S1218 | 198 | 2355 | 424 | CTGACTCA | TGAGAGAC | 0.84 | 0.80 |
| | | | | GTCTCTCA | TGAGTCAG | | |
| S1219 | 199 | 2356 | 425 | TGACTCAG | GTGAGAGA | 1.08 | 1.00 |
| | | | | TCTCTCAC | CTGAGTCA | | |
| S1220 | 200 | 2357 | 426 | GACTCAGT | TGTGAGAG | 0.95 | 0.95 |
| | | | | CTCTCACA | ACTGAGTC | | |
| S1221 | 201 | 2358 | 427 | ACTCAGTC | GTGTGAGA | 0.95 | 0.82 |
| | | | | TCTCACAC | GACTGAGT | | |
| S1222 | 202 | 2359 | 428 | CTCAGTCT | AGTGTGAG | 0.79 | 0.88 |
| | | | | CTCACACT | AGACTGAG | | |
| S1223 | 203 | 2360 | 429 | TCAGTCTC | GAGTGTGA | 0.91 | 0.92 |
| | | | | TCACACTC | GAGACTGA | | |
| S1224 | 204 | 2362 | 430 | AGTCTCTC | ACGAGTGT | 1.02 | 1.05 |
| | | | | ACACTCGT | GAGAGACT | | |
| S1225 | 205 | 2363 | 431 | GTCTCTCA | GACGAGTG | 0.97 | 1.00 |
| | | | | CACTCGTC | TGAGAGAC | | |
| S1226 | 206 | 2364 | 432 | TCTCTCAC | GGACGAGT | 1.16 | 0.96 |
| | | | | ACTCGTCC | GTGAGAGA | | |
| S1227 | 207 | 2699 | 433 | CTCCCACT | TGTCGCAC | 1.13 | 0.93 |
| | | | | GTGCGACA | AGTGGGAG | | |
| S1228 | 208 | 2700 | 434 | TCCCACTG | GTGTCGCA | 1.22 | 0.90 |
| | | | | TGCGACAC | CAGTGGGA | | |
| S1229 | 209 | 2704 | 435 | ACTGTGCG | GAGGGTGT | 0.89 | 0.53 |
| | | | | ACACCCTC | CGCACAGT | | |
| S1230 | 210 | 2743 | 436 | TGATGGAC | ATGGTCTC | 0.68 | 0.92 |
| | | | | GAGACCAT | GTCCATCA | | |
| S1231 | 211 | 2775 | 437 | TACAAATC | CCAGTTCC | 0.79 | 0.78 |
| | | | | GGAACTGG | GATTTGTA | | |
| S1232 | 212 | 2777 | 438 | CAAATCGG | CTCCAGTT | 0.59 | 0.66 |
| | | | | AACTGGAG | CCGATTTG | | |
| S1233 | 213 | 2778 | 439 | AAATCGGA | CCTCCAGT | 0.90 | 0.90 |
| | | | | ACTGGAGG | TCCGATTT | | |
| S1234 | 214 | 2779 | 440 | AATCGGAA | TCCTCCAG | 0.84 | 0.83 |
| | | | | CTGGAGGA | TTCCGATT | | |
| S1235 | 215 | 2780 | 441 | ATCGGAAC | TTCCTCCA | 0.94 | 0.91 |
| | | | | TGGAGGAA | GTTCCGAT | | |
| S1236 | 216 | 2781 | 442 | TCGGAACT | GTTCCTCC | 0.64 | 0.65 |
| | | | | GGAGGAAC | AGTTCCGA | | |
| S1237 | 217 | 3337 | 443 | TCAAGAGC | TCGAACCA | 0.72 | 0.61 |
| | | | | TGGTTCGA | GCTCTTGA | | |
| S1238 | 218 | 3441 | 444 | AGCGACAA | TTCAGTGA | 0.74 | 0.77 |
| | | | | TCACTGAA | TTGTCGCT | | |
| S1239 | 219 | 3442 | 445 | GCGACAAT | GTTCAGTG | 0.68 | 0.78 |
| | | | | CACTGAAC | ATTGTCGC | | |
| S1240 | 220 | 3443 | 446 | CGACAATC | CGTTCAGT | 0.82 | 0.93 |
| | | | | ACTGAACG | GATTGTCG | | |
| S1241 | 221 | 3444 | 447 | GACAATCA | GCGTTCAG | 0.78 | 0.78 |
| | | | | CTGAACGC | TGATTGTC | | |
| S1242 | 222 | 3445 | 448 | ACAATCAC | GGCGTTCA | 0.70 | 0.77 |
| | | | | TGAACGCC | GTGATTGT | | |
| S1243 | 223 | 3447 | 449 | AATCACTG | TCGGCGTT | 0.95 | 0.94 |
| | | | | AACGCCGA | CAGTGATT | | |
| S1244 | 224 | 3448 | 450 | ATCACTGA | TTCGGCGT | 1.03 | 0.97 |
| | | | | ACGCCGAA | TCAGTGAT | | |
| S1245 | 225 | 3449 | 451 | TCACTGAA | CTTCGGCG | 0.86 | 0.87 |
| | | | | CGCCGAAG | TTCAGTGA | | |
| S1246 | 226 | 3453 | 452 | TGAACGCC | CAGGCTTC | 0.92 | 0.94 |
| | | | | GAAGCCTG | GGCGTTCA | | |
| S1247 | 227 | 3455 | 453 | AACGCCGA | TGCAGGCT | 0.80 | 0.70 |
| | | | | AGCCTGCA | TCGGCGTT | | |
| S1248 | 228 | 3582 | 454 | TCACCAAG | GCGTGAAA | 0.37 | 0.76 |
| | | | | TTTCACGC | CTTGGTGA | | |
| S1249 | 229 | 3583 | 455 | CACCAAGT | TGCGTGAA | 0.32 | 0.83 |
| | | | | TTCACGCA | ACTTGGTG | | |

TABLE 4
Selected ASOs targeting APOE pre-mRNA with >30%
APOE mRNA reduction relative to SEQ ID NO: 83

| ASO NO: | SEQ ID NO: |
|---|---|
| S1104 | 84 |
| S1105 | 85 |
| S1108 | 88 |
| S1110 | 90 |
| S1111 | 91 |
| S1114 | 94 |
| S1116 | 96 |
| S1120 | 100 |
| S1123 | 103 |
| S1124 | 104 |
| S1128 | 108 |
| S1131 | 111 |
| S1133 | 113 |
| S1134 | 114 |
| S1135 | 115 |
| S1139 | 119 |
| $1140 | 120 |
| S1143 | 123 |
| S1144 | 124 |
| S1149 | 129 |
| S1150 | 130 |
| S1151 | 131 |
| S1153 | 133 |
| S1154 | 134 |
| S1155 | 135 |
| S1156 | 136 |
| S1158 | 138 |
| S1170 | 150 |
| S1171 | 151 |
| S1177 | 157 |
| S1183 | 163 |
| S1184 | 164 |
| S1188 | 168 |
| S1190 | 170 |
| S1195 | 175 |
| S1196 | 176 |
| S1200 | 180 |
| S1205 | 185 |
| S1230 | 210 |
| S1232 | 212 |
| S1236 | 216 |
| S1239 | 219 |
| S1248 | 228 |
| S1249 | 229 |

TABLE 5
Selected ASOs targeting APOE pre-mRNA with >10%
APOE mRNA knockdown normalized to HPRT1 gene

| ASO NO: | SEQ ID NO: |
|---|---|
| S1147 | 127 |
| S1152 | 132 |
| S1167 | 147 |
| S1222 | 202 |
| S1240 | 220 |

### Example 1: Evaluation of APOE-specific ASO Target Knockdown Efficacy and Toxicities in HEP3B Cells

A detailed description of the methods used in this example can be found in the Supplemental Experimental Section below. Briefly, knockdown efficacy of 79 APOE-specific ASOs was evaluated in human HEP3B cells in vitro. The cells were treated with APOE-specific ASO (12.5 nM) or non-silencing control oligonucleotide (12.5 nM) by transfecting cells with LIPOFECTAMINE™ 2000 (THERMO FISHER SCIENTIFIC INC., 11668030). After 48 hrs. of treatment, cells were lysed. APOE and HPRT1 mRNA levels were analyzed using quantitative real time polymerase chain reaction (qPCR) and the APOE expression values were normalized to values for the endogenous housekeeping control gene HPRT1. The knockdown results of APOE and HRPT1 by ASOs in HEP3B cells are shown as residual APOE or HRPT1 mRNA expression relative to the non-silencing control ASO as the reference sample (normalized to 1) in Table 1.

Treatment of HEP3B cells with the ASOs S1043, S1042, S1044, S1041, and S1025 (SEQ ID NOs: 11, 27-30) achieved >80% knockdown (represented by a residual APOE mRNA expression of <0.2 as compared to cells treated with non-targeting control ASO) (see Table 1)). Of note, not all screened ASOs were highly efficient in downregulating APOE mRNA. Some ASOs, for example ASOs S1017, S1027, S1033, S1018, and S1065 (SEQ ID NOs: 3, 4, 13, 19, and 51) downregulate target gene expression by about 30% as compared to non-targeting control ASO indicating that our screening experiment identified target mRNA knockdown potencies among the 79 tested ASOs ranging from about 30% to about 90% of control reference values.

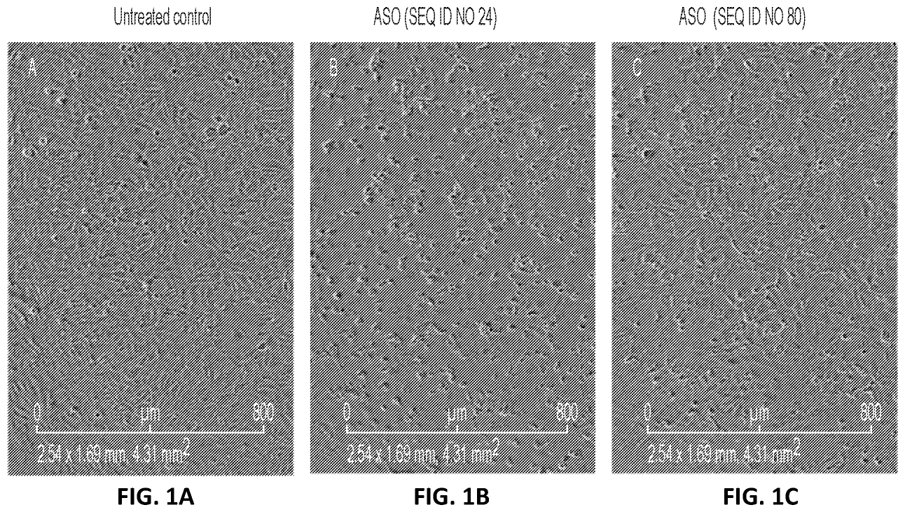

Alteration in the expression level of the HPRT1 housekeeping gene in response to ASO treatment (i.e., transfection) was used as a proxy to identify ASOs that show cellular toxicity under the used experimental conditions. The use of HPRT1 readout as an approximation of cellular toxicity was validated by using live-cell imaging of HEP3B cell cultures. FIGS. 1A, 1B, and 1C show that cultures treated with an ASO S1094 (FIG. 1C, (SEQ ID NO: 80)) that does not downregulate HPRT1 housekeeping gene expression (see Table 1) were non-toxic as determined by images are comparable in confluency and cell morphology to cell cultures not treated with ASO (FIG. 1A). In contrast, cell cultures treated with an ASO (e.g., ASO S1038 (SEQ ID NO: 24)) that downregulates HPRT1 housekeeping gene expression (see Table 1) were not confluent with many cells showing abnormal morphology and dense, pyknotic nuclei indicative of cellular toxicity and death (FIG. 1B). As both ASO S1094 (SEQ ID NO: 80) and S1038 (SEQ ID NO: 24) have comparable efficiencies to downregulate the target gene APOE (see Table 1, about 70 to about 80% downregulation), the observed morphological changes consistent with toxicity and the HPRT1 mRNA are not related to APOE downregulation.

Based on the observation that HPRT1 housekeeping gene downregulation is consistent with confluency and cell morphology (e.g., microscopic indicators of cytotoxicity) the 79 screened molecules were further categorized in a binary fashion. ASOs that reduce HPRT1 mRNA in HEP3B cells more than 20% were considered cytotoxic and vice versa ASO modulating HPRT1 mRNA less than 20% as non-toxic under tested conditions. Table 1 summarizes the HPRT1 mRNA expression levels following transfection of HEP3B cells with ASOs. ASOs S1024, S1088, S1047, S1077, S1059 (SEQ ID NOs: 10, 33, 45, 63, and 74) resulted in a robust APOE target inhibition (e.g., knockdown of APOE mRNA) by >70% without a cytotoxic effect on the expression of HPRT1 housekeeping gene. In contrast, ASOs S1043, S1042, S1044, S1041, and S1025 (SEQ ID NOs: 11, 27, 28, 29, and 30) although very effective in downregulating APOE mRNA, show a cytotoxic effect resulting in a reduction of HPRT1 housekeeping gene expression in HEP3B cells ranging between about 25% to about 40%.

Taken together 37 ASOs were identified (see Table 2) that downregulate APOE target gene mRNA without causing noticeable cellular toxicity (e.g., no change in confluency, no change in morphology compared to non-silencing control) under the experimental conditions (see FIGS. 1A and 1C). Additional assays may be used to illustrate potency and efficacy and other features of these molecules in vitro and in vivo.

### Supplemental Experimental Section

This section describes details on the specific techniques and reagents used in the Example(s) above.

Preparation of Antisense Oligonucleotides (ASOs): The ASOs were synthesized by Integrated DNA Technologies (IDT) using their standard desalting process.

Cell Culture: HEP3B cells (ATCC HB-8064) were maintained at 37° C. and 5% CO₂, in growth medium containing EMEM (ATCC 30-2003) with 10% FBS (VWR 97068-085) and 1% w/w penicillin and 1% w/w streptomycin (Pen Strep) (THERMO FISHER SCIENTIFIC INC. 15140122) and passaged using 0.25% Trypsin-EDTA (THERMO FISHER SCIENTIFIC INC. 25200-114) as cell dissociation reagent. For the ASO screen, HEP3B cells were plated at 15,000 cells per well in 96 well plates in growth medium without Pen Strep.

ASO transfection using Lipofectamine 2000: Each ASO was prepared as a 5 μM stock solution in ULTRAPURE™ DNase/RNase-Free Distilled Water (THERMO FISHER INC., 10977023) and stored at −80° C. until use. Transfections were performed in 96-well plates each at a final ASO concentration of 12.5 nM into 3 culture wells as biological triplicates. One day following cell plating, culture media in each culture well was exchanged with 90 μl growth medium without Pen Strep. For each ASO, the 5 μM stock solution (5 μM) was pre-diluted to 1.25 μM using ULTRAPURE™ DNase/RNase-Free Distilled Water. The pre-diluted ASO was further diluted to 0.25μ M using OPTI-MEM™ (THERMO FISHER INC., 31985062). LIPOFECTAMINE™ 2000 (THERMO FISHER SCIENTIFIC INC., 11668030) was pre-diluted to 6% (v/v) using OPTI-MEM™ and incubated for 5 minutes. Equal volumes of 0.25 UM ASO in OPTI-MEM™ and 6% (v/v) LIPOFECTAMINE™ 2000 (THERMO FISHER SCIENTIFIC INC., 11668030) in OPTI-MEM™ (v/v) were mixed and incubated for 10 minutes. 10 μl of the mixture was added into each culture well, so that the total volume per well was 100 μl. For the no-transfection control, all steps above were the same, except that 0.25 UM ASO in OPTI-MEM™ was replaced by OPTI-MEM™ alone. Cell confluency as a surrogate observational marker to monitor cellular health and potential toxicity of ASO treatments was determined by live-cell imaging of each well at 0 hr, 24 hr and 48 hr post-transfection using INCUCYTE® platform (SARTORIUS AG).

Cell lysis and quantitative RT-PCR: At 48 hr. post-transfection, cells were lysed according to instructions for the CELLS-TO-CT™ 1-step TAQMAN™ Kit (THERMO FISHER SCIENTIFIC, INC. A25602). 50 μl of RNA lysate per culture well was removed and stored at −80° C. until RT-qPCR. 1-step RT-qPCR was carried out in 384-well plates, with each reaction consisting of a 10-μl mixture containing 2.5 μl of TAQMAN™ 1-Step qRT-PCR Mix (THERMO FISHER SCIENTIFIC, INC.), 0.5 μl of APOE TAQMAN™ Assay FAM-MGB, Hs00171168_m1 (THERMO FISHER SCIENTIFIC, INC. 4331182), 0.5 μl of HPRT1 TAQMAN™ Assay VIC-MGB, Hs02800695_m1 (THERMO FISHER SCIENTIFIC, INC. 4448489), 1 μl of RNA lysate, and 5.5 μl of UltraPure™ DNase/RNase-Free Distilled Water. The thermocycler method was as follows: 50° C. for 5 min, 95° C. for 20 sec, followed by 40 cycles of: i) 95° C. for 3 sec, ii) 60° C. for 30 sec. Cycle threshold (Ct) values were analyzed using the delta-delta Ct method, with HRPT1 being the house-keeping gene and either the non-silencing control ASO or the no-transfection control being the reference sample. Variation between culture plates was controlled by including no-transfection control, a non-silencing control ASO, and a positive control identified from a pilot screen (S1087, SEQ ID NO: 73) for APOE knockdown in the transfection scheme of each culture plate. Inter-culture-plate normalization was performed with the “Percentage of positive control” method as described in Malo N, Hanley J A, Cerquozzi S, Pelletier J, Nadon R (2006) Statistical practice in high-throughput screening data analysis. Nat Biotechnol 24:167-175.

### Example 2: Evaluation of APOE-Pre-mRNA Specific ASO Target Knockdown Efficacy and Toxicities in HEP3B Cells

A detailed description of the methods used in this example can be found in the Supplemental Experimental Section below. Unless stated otherwise, all other methods used in this example were analogous to those presented in Example 1 above. Briefly, knockdown efficacy of 147 ASOs targeting APOE pre-mRNA was evaluated in human HEP3B cells in vitro. The cells were treated with ASOs targeting APOE pre-mRNA (5 μM) or non-silencing control oligonucleotide S1250 (5 μM, SEQ ID NO: 83) by gymnotic delivery. Following 72 hours of treatment, cells were lysed. APOE and HPRT1 mRNA levels were analyzed using quantitative real time polymerase chain reaction (qPCR). The knockdown results of APOE and HRPT1 by ASOs targeting APOE pre-mRNA in HEP3B cells are shown as residual APOE or HRPT1 mRNA expression relative to the non-silencing control ASO as the reference sample (normalized to 1) in Table 3.

Treatment of HEP3B cells with the ASOs S1104, S1105, S1108, S1110, $1111, S1114, S1116, S1120, S1123, S1124, S1128, S1131, S1133, S1134, S1135, S1139, S1140, S1143, S1144, S1149, S1150, S1151, S1153, S1154, S1155, S1156, S1158, S1170, S1171, S1177, S1183, S1184, S1188, S1190, S1195, S1196, S1200, S1205, S1230, S1232, S1236, S1239, S1248, and S1249 (SEQ ID NOs: 84, 85, 88, 90, 91, 94, 96, 100, 103, 104, 108, 111, 113, 114, 115, 119, 120, 123, 124, 129, 130, 131, 133, 134, 135, 136, 138, 150, 151, 157, 163, 164, 168, 170, 175, 176, 180, 185, 210, 212, 216, 219, 228, and 229) (e.g., the ASOs of Table 4) achieved >30% knockdown (represented by a residual APOE mRNA expression of <0.7 as compared to cells treated with non-targeting control ASO) (see Table 3)). Treatment of HEP3B cells with the ASOs S1104, S1139, S1140, S1143, S1144, S1149, S1153, S1154, S1155, S1156, S1188, S1205, S1248, and $1249 (SEQ ID NOs: 84, 119, 120, 123, 124, 129, 133, 134, 135, 136, 168, 185, 228, and 229) via gymnotic delivery achieved >50% knockdown (represented by a residual APOE mRNA expression of <0.5 as compared to cells treated with non-targeting control ASO) (see Table 3). Some ASOs S1144, S1153, S1154, S1156, and S1188 (SEQ ID NOs: 124, 133, 134, 136, and 168) resulted in >60% reduction of HPRT1 mRNA as compared to cells treated with non-targeting control ASO. Lack of any visible cell death or morphological change suggests experimental or biological variabilities of HPRT1 mRNA expression with gymnotic ASO delivery.

### Supplemental Experimental Section

This section describes the reagents, protocols, and details regarding the specific techniques used in Example 2.

Treatment with LNA-modified ASO: Each ASO was prepared as a 500 μM stock solution in ULTRAPURE™ DNase/RNase-Free Distilled Water (THERMO FISHER INC., 10977023) and stored at −80° C. until use. Treatments were performed in 96-well plates each at a final ASO concentration of 5 μM into 3 culture wells as biological triplicates. For each ASO, the 500 μM stock solution was pre-diluted to 250 μM using ULTRAPURE™ DNase/RNase-Free Distilled Water. One hour following cell plating, 2 μl of prediluted ASO was added into each culture well. Cell confluency as a surrogate observational marker to monitor cellular health and potential toxicity of ASO treatments was determined by live-cell imaging of each well at 72 hours post-treatment using INCUCYTE® platform (SARTORIUS AG).

Cell lysis and quantitative RT-PCR: At 72 hours post-treatment (gymnotic delivery), cells were lysed according to instructions for the CELLS-TO-CT™ 1-step TAQMAN™ Kit (THERMO FISHER SCIENTIFIC, INC. A25602). 50 μl of RNA lysate per culture well was removed and stored at −80° C. until RT-qPCR. 1-step RT-qPCR was carried out in 384-well plates, with each reaction consisting of a 10-μl mixture containing 2.5 μl of TAQMAN™ 1-Step qRT-PCR Mix (THERMO FISHER SCIENTIFIC, INC.), 0.5 μl of APOE TAQMAN™ Assay FAM-MGB, Hs00171168_m1 (THERMO FISHER SCIENTIFIC, INC. 4331182), 0.5 μl of HPRT1 TAQMAN™ Assay VICMGB, Hs02800695_m1 (THERMO FISHER SCIENTIFIC, INC. 4448489), 1 μl of RNA lysate, and 5.5 μl of UltraPure™ DNase/RNase-Free Distilled Water. The thermocycler method was as follows: 50° C. for 5 min, 95° C. for 20 sec, followed by 40 cycles of: i) 95° C. for 3 sec, ii) 60° C. for 30 sec. Cycle threshold (Ct) values were analyzed using the delta-delta Ct method, with HRPT1 being the house-keeping gene and either the non-silencing control ASO or the treatment control being the reference sample. Variation between culture plates was controlled by including a non-silencing control ASO (S 1250 (SEQ ID NO: 83)), and an assay control ASO (S1248 (SEQ ID NO: 228)) for APOE knockdown of each culture plate. Interculture-plate normalization was performed with the “Percentage of positive control” method as described in Malo N, Hanley J A, Cerquozzi S, Pelletier J, Nadon R (2006) Statistical practice in high-throughput screening data analysis. Nat Biotechnol 24:167-175.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By of example, “an element” means one element or more than one element.

“About” as used herein when referring to a measurable value such as an amount, a temporal duration, and the like, is meant to encompass variations of 20% or 10%, more preferably 5%, even more preferably 1%, and still more preferably 0.1% from the specified value, as such variations are appropriate to perform the disclosed methods.

“Effective amount” or “therapeutically effective amount” are used interchangeably herein, and refer to an amount of a compound, formulation, material, or composition, as described herein effective to achieve a particular biological result or provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

As used herein, the terms “peptide,” “polypeptide,” and “protein” are used interchangeably, and refer to a compound comprises of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “subject” is intended to include living organisms in which an immune response can be elicited (e.g., mammals). A “subject” or “patient” as used herein, may be a human or non-human mammal. Non-human mammals include, for example, livestock and pets, such as ovine, bovine, porcine, canine, feline and murine mammals. Preferably, the subject is human.

Ranges: throughout this disclosure, various aspects of the invention can be presented in a range format. It should be understood that the description in range format is merely for convenience and brevity, and should not be construed as an inflexible limitation on the scope of the invention. Accordingly, the description of a range should be considered to have specifically disclosed all of the possible subranges, as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

1. An antisense oligonucleotide or pharmaceutically acceptable salt thereof comprising a sequence of SEQ ID NO: 5-11, 15, 16, 20-41, 43, 45, 47-50, 53, 55-57, 60-75, or 77-81 or at least 80% sequence identity to SEQ ID NO: 5-11, 15, 16, 20-41, 43, 45, 47-50, 53, 55-57, 60-75, or 77-81.

2-3. (canceled)

4. The antisense oligonucleotide or pharmaceutically acceptable salt thereof of claim 1, wherein the antisense oligonucleotide or pharmaceutically acceptable salt thereof comprises about 15 to about 25 nucleobases.

5-7. (canceled)

8. The antisense oligonucleotide or pharmaceutically acceptable salt thereof of claim 1, wherein the antisense oligonucleotide or pharmaceutically acceptable salt thereof comprises:
(a) one or more inter-nucleoside linkage modifications, wherein the inter-nucleoside linkage modification is selected from the group consisting of a phosphorothioate, a phosphorodithioate, a methyl phosphonate, a methyl phosphorothioate, a phosphoramidate, a phosphorodiamidate, a thiophosphoramidate, a mesyl phosphoramidate, and a combination thereof;
(b) one or more phosphorothioate inter-nucleoside linkages;
(c) DNA, RNA, a protein, a polypeptide, or a small molecule;
(d) one or more modified nucleosides; wherein the modified nucleoside is selected from the group consisting of a 2′-modified nucleoside, a 4′-modified nucleoside, a bridged nucleoside, a phosphorodiamidate morpholine, a locked nucleic acid, an ethylene-bridged nucleic acid, a glycol nucleic acid, a hexitol nucleic acid, a cyclohexene nucleic acid, an arabino nucleic acid, a peptide nucleic acid, a threose nucleic acid, a tricyclo-2′-deoxy-nucleotide, a 1′-deoxyribopentose, a 1′ 2′-dideoxyribopentose, 2′,3′-dideoxyribopentose, 2′,3′-didehydro-2′,3′-dideoxyribopentose, an unlocked nucleic acid, and a combination thereof;
(e) one or more 2′-nucleoside modifications, the 2′-nucleoside modification is selected from the group consisting of a 2′-fluoro-nucleoside, a 2′-O-methyl-nucleoside, a 2′-O-methoxy ethyl nucleoside, a 2′-O-benzyl-2′-deoxynucleoside, a 2′-O-methyl-4-pyridinylnucleoside (2′-O—CH2Py(4)), a 2′-amino-nucleoside, and a combination thereof;
(f) at least eight 2′-nucleoside modifications;
(g) at least ten 2′-nucleoside modifications;
(h) one or more modified nucleobases, wherein the one or more modified nucleobases are selected from a group consisting of S-methyluracil, N6-methyladenine N4-methylcytosine, N7-methylguanine, S-hydroxymethylcytosine N3-methylcytosine N4-acetylcytosine, pseudouridine (Ψ), N1-methyl-pseudouridine (N1-methyl-Ψ), 2-thiouridine (s2U), 5-fluoro-2′-deoxyuridine (FUDR), 8-oxo-7,8-dihydroguanosine (8-oxoG), N-ethylpiperidine-7-EAA triazole modified adenine, N-ethylpiperidine-6-triazole modified adenine, 6-phenylpyrrolo-cytosine (PhpC), 2,4-difluorotoluyl-ribonucleoside (rF), N1 (5-nitroindole) ribonucleoside, 5-methoxyuridine), and a combination thereof; or;
(i) an inverted nucleotide.

9-29. (canceled)

30. The antisense oligonucleotide or pharmaceutically acceptable salt thereof of claim 1, wherein the antisense oligonucleotide or pharmaceutically acceptable salt thereof is a gapmer.

31. The antisense oligonucleotide or pharmaceutically acceptable salt thereof of claim 30, wherein the gapmer comprises:
(a) flanking RNA nucleotides on the 5′-end, the 3′-end, or both;
(b) flanking RNA nucleotides on the 5′-end, the 3′-end, or both, independently ranging from 1 nucleotide to about 9 nucleotides;
(c) about 18 to about 22 nucleosides in length;
(d) ten central 2′-deoxynucleosides and four flanking RNA nucleotides each on both the 5′-end and the 3′-end; or
(e) ten central 2′-deoxynucleosides and/or five flanking ribonucleosides each on both the 5′-end and the 3′-end.

32-37. (canceled)

38. The antisense oligonucleotide or pharmaceutically acceptable salt thereof of claim 1, wherein administration of the antisense oligonucleotide or pharmaceutically acceptable salt thereof to a subject reduces mRNA, pre-mRNA, protein expression, or a combination thereof of human APOE.

39. The antisense oligonucleotide or pharmaceutically acceptable salt thereof of claim 38, wherein human APOE mRNA or human APOE protein expression is reduced by about 25% or greater relative to pretreatment condition.

40-45. (canceled)

46. The antisense oligonucleotide or pharmaceutically acceptable salt thereof of claim 38, wherein the human APOE mRNA comprises a sequence that is at least 80% identical to SEQ ID NO: 1 or encodes a protein comprising a sequence that is 85% identical to SEQ ID NO: 82.

47. (canceled)

48. The antisense oligonucleotide or pharmaceutically acceptable salt thereof of claim 38, wherein the human APOE is an isoform selected from the group consisting of APOE2, APOE3, APOE4, APOE5f, APOE5s, and APOE7.

49. (canceled)

50. A method of treating a disease or disorder in a human subject in need thereof comprising administering the antisense oligonucleotide or pharmaceutically acceptable salt thereof of claim 1 to a subject in need thereof, wherein the disease or disorder comprises a neurodegenerative disease, a tauopathy, an acute or chronic injury, an acute or chronic inflammatory condition in the nervous system or in the body periphery, a vascular disease or a vascular pathological condition, or a lipid storage disorder.

51. (canceled)

52. The method of claim 50, wherein the disease or disorder is selected from the group consisting of Alzheimer's disease, amyotrophic lateral sclerosis, Huntington's disease, Lewy body dementia, multiple sclerosis, multiple system atrophy, Parkinson's disease, prion disease, primary age-related tauopathy, chronic traumatic encephalopathy, progressive supranuclear palsy, corticobasal degeneration, frontotemporal dementia and parkinsonism linked to chromosome 17, vacuolar tauopathy, lytico-bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, behavioral variant frontotemporal dementia (Pick's disease), argyrophilic grain disease, diffuse axonal injuries, concussion, edema, hematoma, skull fractures, hemorrhage, hypoxic brain injury, anoxic brain injury, carbon monoxide poisoning, brain aneurysm, hydrocephalus, traumatic brain injury, spinal cord injury, chemo brain, neuronal damage from tumors, neuronal injury as consequence of meningitis, encephalitis and other inflammation in the body or nervous system, autoimmune disease, stroke, infectious disease, immune response to cancer, a tumor, cerebrovascular disease, diseases caused or influenced by blood-brain barrier leakage, peripheral vascular disease, peripheral artery disease carotid artery disease, pulmonary embolism, abdominal aortic aneurism, collagen vascular disease, chronic venous insufficiency thrombosis, deep vein thrombosis, cerebral small vessel disease, blood-brain barrier leakage, atherosclerosis, cerebral amyloid angiopathy, Cerebrotendineous xanthomatosis, Farber disease, Fabry disease, fucosidosis, Gaucher disease, GM1 gangliosidoses, GM2-gangliosidosis AB variant, Krabbe disease, metachromatic leukodystrophy, multiple sulfatase deficiency, Niemann Pick disease type A, Niemann Pick disease type B, Niemann-Pick disease type C, Sandhoff disease, Schindler disease, Tay-Sachs disease, Wolman disease, and a combination thereof.

53-63. (canceled)

64. The method of claim 50, wherein the antisense oligonucleotide or pharmaceutically acceptable salt thereof is administered to a subject in need thereof, wherein the oligonucleotide or pharmaceutically acceptable salt thereof is administered locally, systemically, orally, sublingually, nasally, subcutaneously, intravenously, intraperitoneally, intramuscularly, intratumorally, intrathecally, intraventricularly, transdermal, rectally, or a combination thereof.

65-67. (canceled)

68. The method of claim 50, wherein the antisense oligonucleotide or pharmaceutically acceptable salt thereof is administered in combination with one or more vaccines, antigens, antibodies, cytotoxic agents, chemotherapeutic agents (both traditional chemotherapy and modem targeted therapies), radiation, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or a combinations thereof.

69-76. (canceled)

77. An antisense oligonucleotide or pharmaceutically acceptable salt thereof comprising a sequence of SEQ ID NO: 84, 85, 88, 90, 91, 94, 96, 100, 103, 104, 108, 111, 113, 114, 115, 119, 120, 123, 124, 129, 130, 131, 133, 134, 135, 136, 138, 150, 151, 157, 163, 164, 168, 170, 175, 176, 180, 185, 210, 212, 216, 219, 228, or 229 or at least 80% sequence identity to SEQ ID NO: 84, 85, 88, 90, 91, 94, 96, 100, 103, 104, 108, 111, 113, 114, 115, 119, 120, 123, 124, 129, 130, 131, 133, 134, 135, 136, 138, 150, 151, 157, 163, 164, 168, 170, 175, 176, 180, 185, 210, 212, 216, 219, 228, or 229.

78-79. (canceled)

80. The antisense oligonucleotide or pharmaceutically acceptable salt thereof of claim 77, wherein the antisense oligonucleotide or pharmaceutically acceptable salt thereof comprises about 12 to about 25 nucleobases.

81-83. (canceled)

84. The antisense oligonucleotide or pharmaceutically acceptable salt thereof of claim 77, wherein the antisense oligonucleotide or pharmaceutically acceptable salt thereof comprises:
(a) one or more inter-nucleoside linkage modifications, the inter-nucleoside linkage modification is selected from the group consisting of a phosphorothioate, a phosphorodithioate, a methyl phosphonate, a methyl phosphorothioate, a phosphoramidate, a phosphorodiamidate, a thiophosphoramidate, a mesyl phosphoramidate, and a combination thereof;
(b) one or more phosphorothioate inter-nucleoside linkages;
(c) DNA, RNA, a protein, a polypeptide, or a small molecule;
(d) one or more modified nucleosides, wherein the one or more modified nucleosides are selected from the group consisting of a 2′-modified nucleoside, a 4′-modified nucleoside, a bridged nucleoside, a phosphorodiamidate morpholine, a locked nucleic acid, an ethylene-bridged nucleic acid, a glycol nucleic acid, a hexitol nucleic acid, a cyclohexene nucleic acid, an arabino nucleic acid, a peptide nucleic acid, a threose nucleic acid, a tricyclo-2′-deoxy-nucleotide, a 1′-deoxyribopentose, a 1′,2′-dideoxyribopentose, 2′,3′-dideoxyribopentose, 2′,3′-didehydro-2′,3′-dideoxyribopentose, an unlocked nucleic acid, and a combination thereof;
(e) one or more 2′-nucleoside modifications, wherein the 2′-nucleoside modification is selected from the group consisting of a 2′-fluoro-nucleoside, a 2′-O-methyl-nucleoside, a 2′-O-methoxy ethyl nucleoside, a 2′-O-benzyl-2′-deoxynucleoside, a 2′-O-methyl-4-pyridinylnucleoside (2′-O—CH2Py(4)), a 2′-amino-nucleoside, and a combination thereof;
(f) at least eight 2′-nucleoside modifications;
(g) at least ten 2′-nucleoside modifications;
(h) one or more modified nucleobases, wherein the one or more modified nucleobases is selected from a group consisting of 5-methyluracil, N6-methyladenine, N4-methylcytosine, N7-methylguanine, 5-hydroxymethylcytosine, N3-methylcytosine, N4-acetylcytosine, pseudouridine (Ψ), N1-methyl-pseudouridine (N1-methyl-Y′), 2-thiouridine (s2U), 5-fluoro-2′-deoxyuridine (FUDR), 8-oxo-7,8-dihydroguanosine (8-oxoG), N-ethylpiperidine-7-EAA triazole modified adenine, N-ethylpiperidine-6-triazole modified adenine, 6-phenylpyrrolo-cytosine (PhpC), 2,4-difluorotoluyl-ribonucleoside (rF), N1 (5-nitroindole) ribonucleoside, 5-methoxyuridine), and a combination thereof; or
(i) an inverted nucleotide.

85-105. (canceled)

106. The antisense oligonucleotide or pharmaceutically acceptable salt thereof of claim 77, wherein the antisense oligonucleotide or pharmaceutically acceptable salt thereof is a gapmer.

107. The antisense oligonucleotide or pharmaceutically acceptable salt thereof of claim 106, wherein the gapmer comprises:
(a) flanking RNA nucleotides on the 5′-end, the 3′-end, or both;
(b) flanking RNA nucleotides on the 5′-end, the 3′-end, or both, independently ranging from 1 nucleotide to about 5 nucleotides;
(c) about 14 to about 22 nucleosides in length;
(d) ten central 2′-deoxynucleosides and three flanking RNA nucleotides each on both the 5′-end and the 3′-end;
(e) ten central 2′-deoxynucleosides and three flanking ribonucleosides each on both the 5′-end and the 3′-end.

108-113. (canceled)

114. The antisense oligonucleotide or pharmaceutically acceptable salt thereof of claim 77, wherein administration of the antisense oligonucleotide or pharmaceutically acceptable salt thereof to a subject reduces mRNA, pre-mRNA, protein expression, or a combination thereof of human APOE.

115. The antisense oligonucleotide or pharmaceutically acceptable salt thereof of claim 114, wherein human APOE pre-mRNA or mRNA is reduced by about 10% or greater relative to pretreatment condition, human APOE protein expression is reduced by about 25% or greater relative to pretreatment condition or a combination thereof.

116-121. (canceled)

122. The antisense oligonucleotide or pharmaceutically acceptable salt thereof of claim 114, wherein the human APOE pre-mRNA comprises a sequence that is at least 80% identical to SEQ ID NO: 230 or encodes for a protein comprising a sequence that is 85% identical to SEQ ID NO: 82.

123. (canceled)

124. The antisense oligonucleotide or pharmaceutically acceptable salt thereof of claim 114, wherein the human APOE is an isoform selected from the group consisting of APOE2, APOE3, APOE4, APOE5f, APOE5s, and APOE7.

125. (canceled)

126. A method of treating a disease or disorder in a human subject in need thereof comprising administering the antisense oligonucleotide or pharmaceutically acceptable salt thereof of claim 77 to a subject in need thereof, wherein the disease or disorder comprises a neurodegenerative disease, a tauopathy, an acute or chronic injury, an acute or chronic inflammatory condition in the nervous system or in the body periphery, a vascular disease or a vascular pathological condition, or a lipid storage disorder.

127. (canceled)

128. The method of claim 126, wherein the disease or disorder is selected from the group consisting of Alzheimer's disease, amyotrophic lateral sclerosis, Huntington's disease, Lewy body dementia, multiple sclerosis, multiple system atrophy, Parkinson's disease, prion disease, primary age-related tauopathy, chronic traumatic encephalopathy, progressive supranuclear palsy, corticobasal degeneration, frontotemporal dementia and parkinsonism linked to chromosome 17, vacuolar tauopathy, lytico-bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, behavioral variant frontotemporal dementia (Pick's disease), argyrophilic grain disease, diffuse axonal injuries, concussion, edema, hematoma, skull fractures, hemorrhage, hypoxic brain injury, anoxic brain injury, carbon monoxide poisoning, brain aneurysm, hydrocephalus, traumatic brain injury, spinal cord injury, chemo brain, neuronal damage from tumors, neuronal injury as consequence of meningitis, encephalitis and other inflammation in the body or nervous system, autoimmune disease, stroke, infectious disease, immune response to cancer, a tumor, cerebrovascular disease, diseases caused or influenced by blood-brain barrier leakage, peripheral vascular disease, peripheral artery disease carotid artery disease, pulmonary embolism, abdominal aortic aneurism, collagen vascular disease, chronic venous insufficiency thrombosis, deep vein thrombosis, cerebral small vessel disease, blood-brain barrier leakage, atherosclerosis, cerebral amyloid angiopathy, Cerebrotendineous xanthomatosis, Farber disease, Fabry disease, fucosidosis, Gaucher disease, GM1 gangliosidoses, GM2-gangliosidosis AB variant, Krabbe disease, metachromatic leukodystrophy, multiple sulfatase deficiency, Niemann Pick disease type A, Niemann Pick disease type B, Niemann-Pick disease type C, Sandhoff disease, Schindler disease, Tay-Sachs disease, Wolman disease, and a combination thereof.

129-139. (canceled)

140. The method of claim 126, wherein the antisense oligonucleotide or pharmaceutically acceptable salt thereof is administered locally to a subject in need thereof, wherein the oligonucleotide or pharmaceutically acceptable salt thereof is administered locally, systemically, orally, sublingually, nasally, subcutaneously, intravenously, intraperitoneally, intramuscularly, intratumorally, intrathecally, intraventricularly, transdermal, rectally, or a combination thereof.

141-143. (canceled)

144. The method of claim 126, wherein the antisense oligonucleotide or pharmaceutically acceptable salt thereof is administered in combination with one or more vaccines, antigens, antibodies, cytotoxic agents, chemotherapeutic agents (both traditional chemotherapy and modem targeted therapies), radiation, kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

145-152. (canceled)


SEQUENCE LISTING (455 sequences)

SEQ ID NO: 1 - RNA, 1166 nt, Homo sapiens
  source 1..1166: mol_type = mRNA
ctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 2 - RNA, 20 nt, synthetic construct
  source 1..20: mol_type = other RNA
cctataggactatccaggaa

SEQ ID NO: 3 - RNA, 20 nt, synthetic construct
  source 1..20: mol_type = other RNA
tgattggccagtcggctcct

SEQ ID NO: 4 - RNA, 20 nt, synthetic construct
  source 1..20: mol_type = other RNA
tgtgattggccagtcggctc

SEQ ID NO: 5 - RNA, 20 nt, synthetic construct
  source 1..20: mol_type = other RNA
ctgtgattggccagtcggct

SEQ ID NO: 6 - RNA, 20 nt, synthetic construct
  source 1..20: mol_type = other RNA
cagcaacgcagcccacagaa

SEQ ID NO: 7 - RNA, 20 nt, synthetic construct
  source 1..20: mol_type = other RNA
atgtgaccagcaacgcagcc

SEQ ID NO: 8 - RNA, 20 nt, synthetic construct
  source 1..20: mol_type = other RNA
tccaccgcttgctccacctt

SEQ ID NO: 9 - RNA, 20 nt, synthetic construct
  source 1..20: mol_type = other RNA
tgtctccaccgcttgctcca

SEQ ID NO: 10 - RNA, 20 nt, synthetic construct
  source 1..20: mol_type = other RNA
tctgtctccaccgcttgctc

SEQ ID NO: 11 - RNA, 20 nt, synthetic construct
  source 1..20: mol_type = other RNA
ctctgtctccaccgcttgct

SEQ ID NO: 12 - RNA, 20 nt, synthetic construct
  source 1..20: mol_type = other RNA
aaaagcgacccagtgccagt

SEQ ID NO: 13 - RNA, 20 nt, synthetic construct
  source 1..20: mol_type = other RNA
caaaagcgacccagtgccag

SEQ ID NO: 14 - RNA, 20 nt, synthetic construct
  source 1..20: mol_type = other RNA
ccaaaagcgacccagtgcca

SEQ ID NO: 15 - RNA, 20 nt, synthetic construct
  source 1..20: mol_type = other RNA
tcccaaaagcgacccagtgc

SEQ ID NO: 16 - RNA, 20 nt, synthetic construct
  source 1..20: mol_type = other RNA
atcccaaaagcgacccagtg

SEQ ID NO: 17 - RNA, 20 nt, synthetic construct
  source 1..20: mol_type = other RNA
aatcccaaaagcgacccagt

SEQ ID NO: 18 - RNA, 20 nt, synthetic construct
  source 1..20: mol_type = other RNA
taatcccaaaagcgacccag

SEQ ID NO: 19 - RNA, 20 nt, synthetic construct
  source 1..20: mol_type = other RNA
gtaatcccaaaagcgaccca

SEQ ID NO: 20 - RNA, 20 nt, synthetic construct
  source 1..20: mol_type = other RNA
ggtaatcccaaaagcgaccc

SEQ ID NO: 21 - RNA, 20 nt, synthetic construct
  source 1..20: mol_type = other RNA
aggtaatcccaaaagcgacc

SEQ ID NO: 22 - RNA, 20 nt, synthetic construct
  source 1..20: mol_type = other RNA
tgcacccagcgcaggtaatc

SEQ ID NO: 23 - RNA, 20 nt, synthetic construct
  source 1..20: mol_type = other RNA
ctgcacccagcgcaggtaat

SEQ ID NO: 24 - RNA, 20 nt, synthetic construct
  source 1..20: mol_type = other RNA
tctgcacccagcgcaggtaa

SEQ ID NO: 25 - RNA, 20 nt, synthetic construct
  source 1..20: mol_type = other RNA
atcagcgccctcagttcctg

SEQ ID NO: 26 - RNA, 20 nt, synthetic construct
  source 1..20: mol_type = other RNA
catcagcgccctcagttcct

SEQ ID NO: 27 - RNA, 20 nt, synthetic construct
  source 1..20: mol_type = other RNA
tccatcagcgccctcagttc

SEQ ID NO: 28 - RNA, 20 nt, synthetic construct
  source 1..20: mol_type = other RNA
gtccatcagcgccctcagtt

SEQ ID NO: 29 - RNA, 20 nt, synthetic construct
  source 1..20: mol_type = other RNA
atggtctcgtccatcagcgc

SEQ ID NO: 30 - RNA, 20 nt, synthetic construct
  source 1..20: mol_type = other RNA
catggtctcgtccatcagcg

SEQ ID NO: 31 - RNA, 20 nt, synthetic construct
  source 1..20: mol_type = other RNA
tcatggtctcgtccatcagc

SEQ ID NO: 32 - RNA, 20 nt, synthetic construct
  source 1..20: mol_type = other RNA
cttcatggtctcgtccatca

SEQ ID NO: 33 - RNA, 20 nt, synthetic construct
  source 1..20: mol_type = other RNA
ccagttccgatttgtaggcc

SEQ ID NO: 34 - RNA, 20 nt, synthetic construct
  source 1..20: mol_type = other RNA
ctccagttccgatttgtagg

SEQ ID NO: 35 - RNA, 20 nt, synthetic construct
  source 1..20: mol_type = other RNA
cctccagttccgatttgtag

SEQ ID NO: 36 - RNA, 20 nt, synthetic construct
  source 1..20: mol_type = other RNA
tcctccagttccgatttgta

SEQ ID NO: 37 - RNA, 20 nt, synthetic construct
  source 1..20: mol_type = other RNA
ttcctccagttccgatttgt

SEQ ID NO: 38 - RNA, 20 nt, synthetic construct
  source 1..20: mol_type = other RNA
gttcctccagttccgatttg

SEQ ID NO: 39 - RNA, 20 nt, synthetic construct
  source 1..20: mol_type = other RNA
tgttcctccagttccgattt

SEQ ID NO: 40 - RNA, 20 nt, synthetic construct
  source 1..20: mol_type = other RNA
ttgttcctccagttccgatt

SEQ ID NO: 41 - RNA, 20 nt, synthetic construct
  source 1..20: mol_type = other RNA
gttgttcctccagttccgat

SEQ ID NO: 42 - RNA, 20 nt, synthetic construct
  source 1..20: mol_type = other RNA
ttacgcagcttgcgcaggtg

SEQ ID NO: 43 - RNA, 20 nt, synthetic construct
  source 1..20: mol_type = other RNA
cttacgcagcttgcgcaggt

SEQ ID NO: 44 - RNA, 20 nt, synthetic construct
  source 1..20: mol_type = other RNA
aggagccgcttacgcagctt

SEQ ID NO: 45 - RNA, 20 nt, synthetic construct
  source 1..20: mol_type = other RNA
tctgcaggtcatcggcatcg

SEQ ID NO: 46 - RNA, 20 nt, synthetic construct
  source 1..20: mol_type = other RNA
ttctgcaggtcatcggcatc

SEQ ID NO: 47 - RNA, 20 nt, synthetic construct
  source 1..20: mol_type = other RNA
cttctgcaggtcatcggcat

SEQ ID NO: 48 - RNA, 20 nt, synthetic construct
  source 1..20: mol_type = other RNA
gcttctgcaggtcatcggca

SEQ ID NO: 49 - RNA, 20 nt, synthetic construct
  source 1..20: mol_type = other RNA
aggcgcttctgcaggtcatc

SEQ ID NO: 50 - RNA, 20 nt, synthetic construct
  source 1..20: mol_type = other RNA
tacactgccaggcgcttctg

SEQ ID NO: 51 - RNA, 20 nt, synthetic construct
  source 1..20: mol_type = other RNA
tggtacactgccaggcgctt

SEQ ID NO: 52 - RNA, 20 nt, synthetic construct
  source 1..20: mol_type = other RNA
ctgctccttcacctcgtcca

SEQ ID NO: 53 - RNA, 20 nt, synthetic construct
  source 1..20: mol_type = other RNA
cacctgctccttcacctcgt

SEQ ID NO: 54 - RNA, 20 nt, synthetic construct
  source 1..20: mol_type = other RNA
tcgaaccagctcttgaggcg

SEQ ID NO: 55 - RNA, 20 nt, synthetic construct
  source 1..20: mol_type = other RNA
ctcgaaccagctcttgaggc

SEQ ID NO: 56 - RNA, 20 nt, synthetic construct
  source 1..20: mol_type = other RNA
gctcgaaccagctcttgagg

SEQ ID NO: 57 - RNA, 20 nt, synthetic construct
  source 1..20: mol_type = other RNA
ggctcgaaccagctcttgag

SEQ ID NO: 58 - RNA, 20 nt, synthetic construct
  source 1..20: mol_type = other RNA
gggctcgaaccagctcttga

SEQ ID NO: 59 - RNA, 20 nt, synthetic construct
  source 1..20: mol_type = other RNA
tgattgtcgctgggcacagg

SEQ ID NO: 60 - RNA, 20 nt, synthetic construct
  source 1..20: mol_type = other RNA
gtgattgtcgctgggcacag

SEQ ID NO: 61 - RNA, 20 nt, synthetic construct
  source 1..20: mol_type = other RNA
agtgattgtcgctgggcaca

SEQ ID NO: 62 - RNA, 20 nt, synthetic construct
  source 1..20: mol_type = other RNA
tcagtgattgtcgctgggca

SEQ ID NO: 63 - RNA, 20 nt, synthetic construct
  source 1..20: mol_type = other RNA
gttcagtgattgtcgctggg

SEQ ID NO: 64 - RNA, 20 nt, synthetic construct
  source 1..20: mol_type = other RNA
cgttcagtgattgtcgctgg

SEQ ID NO: 65 - RNA, 20 nt, synthetic construct
  source 1..20: mol_type = other RNA
gcgttcagtgattgtcgctg

SEQ ID NO: 66 - RNA, 20 nt, synthetic construct
  source 1..20: mol_type = other RNA
ggcgttcagtgattgtcgct

SEQ ID NO: 67 - RNA, 20 nt, synthetic construct
  source 1..20: mol_type = other RNA
cggcgttcagtgattgtcgc

SEQ ID NO: 68 - RNA, 20 nt, synthetic construct
  source 1..20: mol_type = other RNA
tcggcgttcagtgattgtcg

SEQ ID NO: 69 - RNA, 20 nt, synthetic construct
  source 1..20: mol_type = other RNA
ttcggcgttcagtgattgtc

SEQ ID NO: 70 - RNA, 20 nt, synthetic construct
  source 1..20: mol_type = other RNA
cttcggcgttcagtgattgt

SEQ ID NO: 71 - RNA, 20 nt, synthetic construct
  source 1..20: mol_type = other RNA
gcttcggcgttcagtgattg

SEQ ID NO: 72 - RNA, 20 nt, synthetic construct
  source 1..20: mol_type = other RNA
aggcttcggcgttcagtgat

SEQ ID NO: 73 - RNA, 20 nt, synthetic construct
  source 1..20: mol_type = other RNA
caggcttcggcgttcagtga

SEQ ID NO: 74 - RNA, 20 nt, synthetic construct
  source 1..20: mol_type = other RNA
tgcaggcttcggcgttcagt

SEQ ID NO: 75 - RNA, 20 nt, synthetic construct
  source 1..20: mol_type = other RNA
aactagggtccaccccagga

SEQ ID NO: 76 - RNA, 20 nt, synthetic construct
  source 1..20: mol_type = other RNA
taaactagggtccaccccag

SEQ ID NO: 77 - RNA, 20 nt, synthetic construct
  source 1..20: mol_type = other RNA
ttaaactagggtccacccca

SEQ ID NO: 78 - RNA, 20 nt, synthetic construct
  source 1..20: mol_type = other RNA
cgtgaaacttggtgaatctt

SEQ ID NO: 79 - RNA, 20 nt, synthetic construct
  source 1..20: mol_type = other RNA
gcgtgaaacttggtgaatct

SEQ ID NO: 80 - RNA, 20 nt, synthetic construct
  source 1..20: mol_type = other RNA
tgcgtgaaacttggtgaatc

SEQ ID NO: 81 - RNA, 18 nt, synthetic construct
  source 1..18: mol_type = other RNA
cggccgcgcacgtcctcc

SEQ ID NO: 82 - protein, 300 aa, Homo sapiens
MKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 83 - RNA, 16 nt, synthetic construct
  source 1..16: mol_type = other RNA
ggctactacgccgtca

SEQ ID NO: 84 - RNA, 16 nt, synthetic construct
  source 1..16: mol_type = other RNA
actgcgcttctcaccg

SEQ ID NO: 85 - RNA, 16 nt, synthetic construct
  source 1..16: mol_type = other RNA
tcccagctctttctag

SEQ ID NO: 86 - RNA, 16 nt, synthetic construct
  source 1..16: mol_type = other RNA
tctcctgagactacct

SEQ ID NO: 87 - RNA, 16 nt, synthetic construct
  source 1..16: mol_type = other RNA
ttacatcccagtccag

SEQ ID NO: 88 - RNA, 16 nt, synthetic construct
  source 1..16: mol_type = other RNA
cttacatcccagtcca

SEQ ID NO: 89 - RNA, 16 nt, synthetic construct
  source 1..16: mol_type = other RNA
tggcttacatcccagt

SEQ ID NO: 90 - RNA, 16 nt, synthetic construct
  source 1..16: mol_type = other RNA
atggcttacatcccag

SEQ ID NO: 91 - RNA, 16 nt, synthetic construct
  source 1..16: mol_type = other RNA
ctatggcttacatccc

SEQ ID NO: 92 - RNA, 16 nt, synthetic construct
  source 1..16: mol_type = other RNA
gctatggcttacatcc

SEQ ID NO: 93 - RNA, 16 nt, synthetic construct
  source 1..16: mol_type = other RNA
agtgacaactcgtgga

SEQ ID NO: 94 - RNA, 16 nt, synthetic construct
  source 1..16: mol_type = other RNA
tagtgacaactcgtgg

SEQ ID NO: 95 - RNA, 16 nt, synthetic construct
  source 1..16: mol_type = other RNA
tgatagtgacaactcg

SEQ ID NO: 96 - RNA, 16 nt, synthetic construct
  source 1..16: mol_type = other RNA
cagtaggtgctcgata

SEQ ID NO: 97 - RNA, 16 nt, synthetic construct
  source 1..16: mol_type = other RNA
agatctgaggacactg

SEQ ID NO: 98 - RNA, 16 nt, synthetic construct
  source 1..16: mol_type = other RNA
gagatctgaggacact

SEQ ID NO: 99 - RNA, 16 nt, synthetic construct
  source 1..16: mol_type = other RNA
tggagatctgaggaca

SEQ ID NO: 100 - RNA, 16 nt, synthetic construct
  source 1..16: mol_type = other RNA
atggagatctgaggac

SEQ ID NO: 101 - RNA, 16 nt, synthetic construct
  source 1..16: mol_type = other RNA
cccagttatggagatc

SEQ ID NO: 102 - RNA, 16 nt, synthetic construct
  source 1..16: mol_type = other RNA
agctaccgtgtcgctg

SEQ ID NO: 103 - RNA, 16 nt, synthetic construct
  source 1..16: mol_type = other RNA
tagctaccgtgtcgct

SEQ ID NO: 104 - RNA, 16 nt, synthetic construct
  source 1..16: mol_type = other RNA
ggctagctaccgtgtc

SEQ ID NO: 105 - RNA, 16 nt, synthetic construct
  source 1..16: mol_type = other RNA
atcgacggctagctac

SEQ ID NO: 106 - RNA, 16 nt, synthetic construct
  source 1..16: mol_type = other RNA
aatcgacggctagcta

SEQ ID NO: 107 - RNA, 16 nt, synthetic construct
  source 1..16: mol_type = other RNA
caatcgacggctagct

SEQ ID NO: 108 - RNA, 16 nt, synthetic construct
  source 1..16: mol_type = other RNA
tccaatcgacggctag

SEQ ID NO: 109 - RNA, 16 nt, synthetic construct
  source 1..16: mol_type = other RNA
agttctccaatcgacg

SEQ ID NO: 110 - RNA, 16 nt, synthetic construct
  source 1..16: mol_type = other RNA
aagttctccaatcgac

SEQ ID NO: 111 - RNA, 16 nt, synthetic construct
  source 1..16: mol_type = other RNA
cgccgtgttccattta

SEQ ID NO: 112 - RNA, 16 nt, synthetic construct
  source 1..16: mol_type = other RNA
taagcgccgtgttcca

SEQ ID NO: 113 - RNA, 16 nt, synthetic construct
  source 1..16: mol_type = other RNA
ttaagcgccgtgttcc

SEQ ID NO: 114 - RNA, 16 nt, synthetic construct
  source 1..16: mol_type = other RNA
gttaagcgccgtgttc

SEQ ID NO: 115 - RNA, 16 nt, synthetic construct
  source 1..16: mol_type = other RNA
agttaagcgccgtgtt

SEQ ID NO: 116 - RNA, 16 nt, synthetic construct
  source 1..16: mol_type = other RNA
cagttaagcgccgtgt

SEQ ID NO: 117 - RNA, 16 nt, synthetic construct
  source 1..16: mol_type = other RNA
cacagttaagcgccgt

SEQ ID NO: 118 - RNA, 16 nt, synthetic construct
  source 1..16: mol_type = other RNA
tcacagttaagcgccg

SEQ ID NO: 119 - RNA, 16 nt, synthetic construct
  source 1..16: mol_type = other RNA
ctcacagttaagcgcc

SEQ ID NO: 120 - RNA, 16 nt, synthetic construct
  source 1..16: mol_type = other RNA
cctcacagttaagcgc

SEQ ID NO: 121 - RNA, 16 nt, synthetic construct
  source 1..16: mol_type = other RNA
acctcacagttaagcg

SEQ ID NO: 122 - RNA, 16 nt, synthetic construct
  source 1..16: mol_type = other RNA
caacctcacagttaag

SEQ ID NO: 123 - RNA, 16 nt, synthetic construct
  source 1..16: mol_type = other RNA
ctccaacctcacagtt

SEQ ID NO: 124 - RNA, 16 nt, synthetic construct
  source 1..16: mol_type = other RNA
aagctccaacctcaca

SEQ ID NO: 125 - RNA, 16 nt, synthetic construct
  source 1..16: mol_type = other RNA
taagctccaacctcac

SEQ ID NO: 126 - RNA, 16 nt, synthetic construct
  source 1..16: mol_type = other RNA
ctaagctccaacctca

SEQ ID NO: 127 - RNA, 16 nt, synthetic construct
  source 1..16: mol_type = other RNA
tctaagctccaacctc

SEQ ID NO: 128 - RNA, 16 nt, synthetic construct
  source 1..16: mol_type = other RNA
tcctcattctcccttc

SEQ ID NO: 129 - RNA, 16 nt, synthetic construct
  source 1..16: mol_type = other RNA
cattcctcattctccc

SEQ ID NO: 130 - RNA, 16 nt, synthetic construct
  source 1..16: mol_type = other RNA
gcattcctcattctcc

SEQ ID NO: 131 - RNA, 16 nt, synthetic construct
  source 1..16: mol_type = other RNA
cgcattcctcattctc

SEQ ID NO: 132 - RNA, 16 nt, synthetic construct
  source 1..16: mol_type = other RNA
ctcgcattcctcattc

SEQ ID NO: 133 - RNA, 16 nt, synthetic construct
  source 1..16: mol_type = other RNA
gtcggcctccatagaa

SEQ ID NO: 134 - RNA, 16 nt, synthetic construct
  source 1..16: mol_type = other RNA
tcaaccgccagtgagg

SEQ ID NO: 135 - RNA, 16 nt, synthetic construct
  source 1..16: mol_type = other RNA
atcaaccgccagtgag

SEQ ID NO: 136 - RNA, 16 nt, synthetic construct
  source 1..16: mol_type = other RNA
gggaaggagaaactgt

SEQ ID NO: 137 - RNA, 16 nt, synthetic construct
  source 1..16: mol_type = other RNA
tgaggtgaggatgaga

SEQ ID NO: 138 - RNA, 16 nt, synthetic construct
  source 1..16: mol_type = other RNA
ttgaggtgaggatgag

SEQ ID NO: 139 - RNA, 16 nt, synthetic construct
  source 1..16: mol_type = other RNA
gttgaggtgaggatga

SEQ ID NO: 140 - RNA, 16 nt, synthetic construct
  source 1..16: mol_type = other RNA
aggttgaggtgaggat

SEQ ID NO: 141 - RNA, 16 nt, synthetic construct
  source 1..16: mol_type = other RNA
agaagcctcagaagag

SEQ ID NO: 142 - RNA, 16 nt, synthetic construct
  source 1..16: mol_type = other RNA
cagaagcctcagaaga

SEQ ID NO: 143 - RNA, 16 nt, synthetic construct
  source 1..16: mol_type = other RNA
acagaagcctcagaag

SEQ ID NO: 144 - RNA, 16 nt, synthetic construct
  source 1..16: mol_type = other RNA
cacagaagcctcagaa

SEQ ID NO: 145 - RNA, 16 nt, synthetic construct
  source 1..16: mol_type = other RNA
gagagcgtcaaatcgc

SEQ ID NO: 146 - RNA, 16 nt, synthetic construct
  source 1..16: mol_type = other RNA
agagagcgtcaaatcg

SEQ ID NO: 147 - RNA, 16 nt, synthetic construct
  source 1..16: mol_type = other RNA
cagagagcgtcaaatc

SEQ ID NO: 148 - RNA, 16 nt, synthetic construct
  source 1..16: mol_type = other RNA
ccagagagcgtcaaat

SEQ ID NO: 149 - RNA, 16 nt, synthetic construct
  source 1..16: mol_type = other RNA
cccagagagcgtcaaa

SEQ ID NO: 150 - RNA, 16 nt, synthetic construct
  source 1..16: mol_type = other RNA
ctatctcaaggatggg

SEQ ID NO: 151 - RNA, 16 nt, synthetic construct
  source 1..16: mol_type = other RNA
cctatctcaaggatgg

SEQ ID NO: 152 - RNA, 16 nt, synthetic construct
  source 1..16: mol_type = other RNA
atcgaaacgggcagat

SEQ ID NO: 153 - RNA, 16 nt, synthetic construct
  source 1..16: mol_type = other RNA
gatcgaaacgggcaga

SEQ ID NO: 154 - RNA, 16 nt, synthetic construct
  source 1..16: mol_type = other RNA
agatcgaaacgggcag

SEQ ID NO: 155 - RNA, 16 nt, synthetic construct
  source 1..16: mol_type = other RNA
gagatcgaaacgggca

SEQ ID NO: 156 - RNA, 16 nt, synthetic construct
  source 1..16: mol_type = other RNA
ggagatcgaaacgggc

SEQ ID NO: 157 - RNA, 16 nt, synthetic construct
  source 1..16: mol_type = other RNA
tgggagatcgaaacgg

SEQ ID NO: 158 - RNA, 16 nt, synthetic construct
  source 1..16: mol_type = other RNA
ttgggagatcgaaacg

SEQ ID NO: 159 - RNA, 16 nt, synthetic construct
  source 1..16: mol_type = other RNA
tttgggagatcgaaac

SEQ ID NO: 160 - RNA, 16 nt, synthetic construct
  source 1..16: mol_type = other RNA
ctttgggagatcgaaa

SEQ ID NO: 161 - RNA, 16 nt, synthetic construct
  source 1..16: mol_type = other RNA
aacctctaactcccag

SEQ ID NO: 162 - RNA, 16 nt, synthetic construct
  source 1..16: mol_type = other RNA
cgtgtctggtattcac

SEQ ID NO: 163 - RNA, 16 nt, synthetic construct
  source 1..16: mol_type = other RNA
ccgtgtctggtattca

SEQ ID NO: 164 - RNA, 16 nt, synthetic construct
  source 1..16: mol_type = other RNA
tgagtgtccttgtgtg

SEQ ID NO: 165 - RNA, 16 nt, synthetic construct
  source 1..16: mol_type = other RNA
aaccttgaccttctgg

SEQ ID NO: 166 - RNA, 16 nt, synthetic construct
  source 1..16: mol_type = other RNA
caaccttgaccttctg

SEQ ID NO: 167 - RNA, 16 nt, synthetic construct
  source 1..16: mol_type = other RNA
gcaaccttgaccttct

SEQ ID NO: 168 - RNA, 16 nt, synthetic construct
  source 1..16: mol_type = other RNA
tgcaaccttgaccttc

SEQ ID NO: 169 - RNA, 16 nt, synthetic construct
  source 1..16: mol_type = other RNA
ggcctgaacatggttc

SEQ ID NO: 170 - RNA, 16 nt, synthetic construct
  source 1..16: mol_type = other RNA
gagtcggtttaatcac

SEQ ID NO: 171 - RNA, 16 nt, synthetic construct
  source 1..16: mol_type = other RNA
tctttggagccatggt

SEQ ID NO: 172 - RNA, 16 nt, synthetic construct
  source 1..16: mol_type = other RNA
ttctttggagccatgg

SEQ ID NO: 173 - RNA, 16 nt, synthetic construct
  source 1..16: mol_type = other RNA
cttctttggagccatg

SEQ ID NO: 174 - RNA, 16 nt, synthetic construct
  source 1..16: mol_type = other RNA
gaaggtgctccacaaa

SEQ ID NO: 175 - RNA, 16 nt, synthetic construct
  source 1..16: mol_type = other RNA
catctagtacctaggg

SEQ ID NO: 176 - RNA, 16 nt, synthetic construct
  source 1..16: mol_type = other RNA
gcatctagtacctagg

SEQ ID NO: 177 - RNA, 16 nt, synthetic construct
  source 1..16: mol_type = other RNA
ggcatctagtacctag

SEQ ID NO: 178 - RNA, 16 nt, synthetic construct
  source 1..16: mol_type = other RNA
caggcatctagtacct

SEQ ID NO: 179 - RNA, 16 nt, synthetic construct
  source 1..16: mol_type = other RNA
tccaggcatctagtac

SEQ ID NO: 180 - RNA, 16 nt, synthetic construct
  source 1..16: mol_type = other RNA
gtccaggcatctagta

SEQ ID NO: 181 - RNA, 16 nt, synthetic construct
  source 1..16: mol_type = other RNA
cgtccaggcatctagt

SEQ ID NO: 182 - RNA, 16 nt, synthetic construct
  source 1..16: mol_type = other RNA
gtcagggtccttctga

SEQ ID NO: 183 - RNA, 16 nt, synthetic construct
  source 1..16: mol_type = other RNA
cctgtgtggaacaagt

SEQ ID NO: 184 - RNA, 16 nt, synthetic construct
  source 1..16: mol_type = other RNA
tcctgtgtggaacaag

SEQ ID NO: 185 - RNA, 16 nt, synthetic construct
  source 1..16: mol_type = other RNA
ctgtctccaccgcttg

SEQ ID NO: 186 - RNA, 16 nt, synthetic construct
  source 1..16: mol_type = other RNA
actcacctcagttcct

SEQ ID NO: 187 - RNA, 16 nt, synthetic construct
  source 1..16: mol_type = other RNA
acactcacctcagttc

SEQ ID NO: 188 - RNA, 16 nt, synthetic construct
  source 1..16: mol_type = other RNA
gacactcacctcagtt

SEQ ID NO: 189 - RNA, 16 nt, synthetic construct
  source 1..16: mol_type = other RNA
atgaaacctggacctg

SEQ ID NO: 190 - RNA, 16 nt, synthetic construct
  source 1..16: mol_type = other RNA
aagacttagcgacagg

SEQ ID NO: 191 - RNA, 16 nt, synthetic construct
  source 1..16: mol_type = other RNA
caagacttagcgacag

SEQ ID NO: 192 - RNA, 16 nt, synthetic construct
  source 1..16: mol_type = other RNA
ccaagacttagcgaca

SEQ ID NO: 193 - RNA, 16 nt, synthetic construct
  source 1..16: mol_type = other RNA
cccaagacttagcgac

SEQ ID NO: 194 - RNA, 16 nt, synthetic construct
  source 1..16: mol_type = other RNA
tagaaccagcagagac

SEQ ID NO: 195 - RNA, 16 nt, synthetic construct
  source 1..16: mol_type = other RNA
ctagaaccagcagaga

SEQ ID NO: 196 - RNA, 16 nt, synthetic construct
  source 1..16: mol_type = other RNA
tgagtcagaagggaag

SEQ ID NO: 197 - RNA, 16 nt, synthetic construct
  source 1..16: mol_type = other RNA
ctgagtcagaagggaa

SEQ ID NO: 198 - RNA, 16 nt, synthetic construct
  source 1..16: mol_type = other RNA
tgagagactgagtcag

SEQ ID NO: 199 - RNA, 16 nt, synthetic construct
  source 1..16: mol_type = other RNA
gtgagagactgagtca

SEQ ID NO: 200 - RNA, 16 nt, synthetic construct
  source 1..16: mol_type = other RNA
tgtgagagactgagtc

SEQ ID NO: 201 - RNA, 16 nt, synthetic construct
  source 1..16: mol_type = other RNA
gtgtgagagactgagt

SEQ ID NO: 202 - RNA, 16 nt, synthetic construct
  source 1..16: mol_type = other RNA
agtgtgagagactgag

SEQ ID NO: 203 - RNA, 16 nt, synthetic construct
  source 1..16: mol_type = other RNA
gagtgtgagagactga

SEQ ID NO: 204 - RNA, 16 nt, synthetic construct
  source 1..16: mol_type = other RNA
acgagtgtgagagact

SEQ ID NO: 205 - RNA, 16 nt, synthetic construct
  source 1..16: mol_type = other RNA
gacgagtgtgagagac

SEQ ID NO: 206 - RNA, 16 nt, synthetic construct
  source 1..16: mol_type = other RNA
ggacgagtgtgagaga

SEQ ID NO: 207 - RNA, 16 nt, synthetic construct
  source 1..16: mol_type = other RNA
tgtcgcacagtgggag

SEQ ID NO: 208 - RNA, 16 nt, synthetic construct
  source 1..16: mol_type = other RNA
gtgtcgcacagtggga

SEQ ID NO: 209 - RNA, 16 nt, synthetic construct
  source 1..16: mol_type = other RNA
gagggtgtcgcacagt

SEQ ID NO: 210 - RNA, 16 nt, synthetic construct
  source 1..16: mol_type = other RNA
atggtctcgtccatca

SEQ ID NO: 211 - RNA, 16 nt, synthetic construct
  source 1..16: mol_type = other RNA
ccagttccgatttgta

SEQ ID NO: 212 - RNA, 16 nt, synthetic construct
  source 1..16: mol_type = other RNA
ctccagttccgatttg

SEQ ID NO: 213 - RNA, 16 nt, synthetic construct
  source 1..16: mol_type = other RNA
cctccagttccgattt

SEQ ID NO: 214 - RNA, 16 nt, synthetic construct
  source 1..16: mol_type = other RNA
tcctccagttccgatt

SEQ ID NO: 215 - RNA, 16 nt, synthetic construct
  source 1..16: mol_type = other RNA
ttcctccagttccgat

SEQ ID NO: 216 - RNA, 16 nt, synthetic construct
  source 1..16: mol_type = other RNA
gttcctccagttccga

SEQ ID NO: 217 - RNA, 16 nt, synthetic construct
  source 1..16: mol_type = other RNA
tcgaaccagctcttga

SEQ ID NO: 218 - RNA, 16 nt, synthetic construct
  source 1..16: mol_type = other RNA
ttcagtgattgtcgct

SEQ ID NO: 219 - RNA, 16 nt, synthetic construct
  source 1..16: mol_type = other RNA
gttcagtgattgtcgc

SEQ ID NO: 220 - RNA, 16 nt, synthetic construct
  source 1..16: mol_type = other RNA
cgttcagtgattgtcg

SEQ ID NO: 221 - RNA, 16 nt, synthetic construct
  source 1..16: mol_type = other RNA
gcgttcagtgattgtc

SEQ ID NO: 222 - RNA, 16 nt, synthetic construct
  source 1..16: mol_type = other RNA
ggcgttcagtgattgt

SEQ ID NO: 223 - RNA, 16 nt, synthetic construct
  source 1..16: mol_type = other RNA
tcggcgttcagtgatt

SEQ ID NO: 224 - RNA, 16 nt, synthetic construct
  source 1..16: mol_type = other RNA
ttcggcgttcagtgat

SEQ ID NO: 225 - RNA, 16 nt, synthetic construct
  source 1..16: mol_type = other RNA
cttcggcgttcagtga

SEQ ID NO: 226 - RNA, 16 nt, synthetic construct
  source 1..16: mol_type = other RNA
caggcttcggcgttca

SEQ ID NO: 227 - RNA, 16 nt, synthetic construct
  source 1..16: mol_type = other RNA
tgcaggcttcggcgtt

SEQ ID NO: 228 - RNA, 16 nt, synthetic construct
  source 1..16: mol_type = other RNA
gcgtgaaacttggtga

SEQ ID NO: 229 - RNA, 16 nt, synthetic construct
  source 1..16: mol_type = other RNA
tgcgtgaaacttggtg

SEQ ID NO: 230 - DNA, 3598 bp, Homo sapiens
  source 1..3598: mol_type = genomic DNA
ctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 231 - RNA, 20 nt, Homo sapiens
  source 1..20: mol_type = mRNA
aggagccgactggccaatca

SEQ ID NO: 232 - RNA, 20 nt, Homo sapiens
  source 1..20: mol_type = mRNA
gagccgactggccaatcaca

SEQ ID NO: 233 - RNA, 20 nt, Homo sapiens
  source 1..20: mol_type = mRNA
agccgactggccaatcacag

SEQ ID NO: 234 - RNA, 20 nt, Homo sapiens
  source 1..20: mol_type = mRNA
ttctgtgggctgcgttgctg

SEQ ID NO: 235 - RNA, 20 nt, Homo sapiens
  source 1..20: mol_type = mRNA
ggctgcgttgctggtcacat

SEQ ID NO: 236 - RNA, 20 nt, Homo sapiens
  source 1..20: mol_type = mRNA
aaggtggagcaagcggtgga

SEQ ID NO: 237 - RNA, 20 nt, Homo sapiens
  source 1..20: mol_type = mRNA
tggagcaagcggtggagaca

SEQ ID NO: 238 - RNA, 20 nt, Homo sapiens
  source 1..20: mol_type = mRNA
gagcaagcggtggagacaga

SEQ ID NO: 239 - RNA, 20 nt, Homo sapiens
  source 1..20: mol_type = mRNA
agcaagcggtggagacagag

SEQ ID NO: 240 - RNA, 20 nt, Homo sapiens
  source 1..20: mol_type = mRNA
actggcactgggtcgctttt

SEQ ID NO: 241 - RNA, 20 nt, Homo sapiens
  source 1..20: mol_type = mRNA
ctggcactgggtcgcttttg

SEQ ID NO: 242 - RNA, 20 nt, Homo sapiens
  source 1..20: mol_type = mRNA
tggcactgggtcgcttttgg

SEQ ID NO: 243 - RNA, 20 nt, Homo sapiens
  source 1..20: mol_type = mRNA
gcactgggtcgcttttggga

SEQ ID NO: 244 - RNA, 20 nt, Homo sapiens
  source 1..20: mol_type = mRNA
cactgggtcgcttttgggat

SEQ ID NO: 245 - RNA, 20 nt, Homo sapiens
  source 1..20: mol_type = mRNA
actgggtcgcttttgggatt

SEQ ID NO: 246 - RNA, 20 nt, Homo sapiens
  source 1..20: mol_type = mRNA
ctgggtcgcttttgggatta

SEQ ID NO: 247 - RNA, 20 nt, Homo sapiens
  source 1..20: mol_type = mRNA
tgggtcgcttttgggattac

SEQ ID NO: 248 - RNA, 20 nt, Homo sapiens
  source 1..20: mol_type = mRNA
gggtcgcttttgggattacc

SEQ ID NO: 249 - RNA, 20 nt, Homo sapiens
  source 1..20: mol_type = mRNA
ggtcgcttttgggattacct

SEQ ID NO: 250 - RNA, 20 nt, Homo sapiens
  source 1..20: mol_type = mRNA
gattacctgcgctgggtgca

SEQ ID NO: 251 - RNA, 20 nt, Homo sapiens
  source 1..20: mol_type = mRNA
attacctgcgctgggtgcag

SEQ ID NO: 252 - RNA, 20 nt, Homo sapiens
  source 1..20: mol_type = mRNA
ttacctgcgctgggtgcaga

SEQ ID NO: 253 - RNA, 20 nt, Homo sapiens
  source 1..20: mol_type = mRNA
caggaactgagggcgctgat

SEQ ID NO: 254 - RNA, 20 nt, Homo sapiens
  source 1..20: mol_type = mRNA
aggaactgagggcgctgatg

SEQ ID NO: 255 - RNA, 20 nt, Homo sapiens
  source 1..20: mol_type = mRNA
gaactgagggcgctgatgga

SEQ ID NO: 256 - RNA, 20 nt, Homo sapiens
  source 1..20: mol_type = mRNA
aactgagggcgctgatggac

SEQ ID NO: 257 - RNA, 20 nt, Homo sapiens
  source 1..20: mol_type = mRNA
gcgctgatggacgagaccat

SEQ ID NO: 258 - RNA, 20 nt, Homo sapiens
  source 1..20: mol_type = mRNA
cgctgatggacgagaccatg

SEQ ID NO: 259 - RNA, 20 nt, Homo sapiens
  source 1..20: mol_type = mRNA
gctgatggacgagaccatga

SEQ ID NO: 260 - RNA, 20 nt, Homo sapiens
  source 1..20: mol_type = mRNA
tgatggacgagaccatgaag

SEQ ID NO: 261 - RNA, 20 nt, Homo sapiens
  source 1..20: mol_type = mRNA
ggcctacaaatcggaactgg

SEQ ID NO: 262 - RNA, 20 nt, Homo sapiens
  source 1..20: mol_type = mRNA
cctacaaatcggaactggag

SEQ ID NO: 263 - RNA, 20 nt, Homo sapiens
  source 1..20: mol_type = mRNA
ctacaaatcggaactggagg

SEQ ID NO: 264 - RNA, 20 nt, Homo sapiens
  source 1..20: mol_type = mRNA
tacaaatcggaactggagga

SEQ ID NO: 265 - RNA, 20 nt, Homo sapiens
  source 1..20: mol_type = mRNA
acaaatcggaactggaggaa

SEQ ID NO: 266 - RNA, 20 nt, Homo sapiens
  source 1..20: mol_type = mRNA
caaatcggaactggaggaac

SEQ ID NO: 267 - RNA, 20 nt, Homo sapiens
  source 1..20: mol_type = mRNA
aaatcggaactggaggaaca

SEQ ID NO: 268 - RNA, 20 nt, Homo sapiens
  source 1..20: mol_type = mRNA
aatcggaactggaggaacaa

SEQ ID NO: 269 - RNA, 20 nt, Homo sapiens
  source 1..20: mol_type = mRNA
atcggaactggaggaacaac

SEQ ID NO: 270 - RNA, 20 nt, Homo sapiens
  source 1..20: mol_type = mRNA
cacctgcgcaagctgcgtaa

SEQ ID NO: 271 - RNA, 20 nt, Homo sapiens
  source 1..20: mol_type = mRNA
acctgcgcaagctgcgtaag

SEQ ID NO: 272 - RNA, 20 nt, Homo sapiens
  source 1..20: mol_type = mRNA
aagctgcgtaagcggctcct

SEQ ID NO: 273 - RNA, 20 nt, Homo sapiens
  source 1..20: mol_type = mRNA
cgatgccgatgacctgcaga

SEQ ID NO: 274 - RNA, 20 nt, Homo sapiens
  source 1..20: mol_type = mRNA
gatgccgatgacctgcagaa

SEQ ID NO: 275 - RNA, 20 nt, Homo sapiens
  source 1..20: mol_type = mRNA
atgccgatgacctgcagaag

SEQ ID NO: 276 - RNA, 20 nt, Homo sapiens
  source 1..20: mol_type = mRNA
tgccgatgacctgcagaagc

SEQ ID NO: 277 - RNA, 20 nt, Homo sapiens
  source 1..20: mol_type = mRNA
gatgacctgcagaagcgcct

SEQ ID NO: 278 - RNA, 20 nt, Homo sapiens
  source 1..20: mol_type = mRNA
cagaagcgcctggcagtgta

SEQ ID NO: 279 - RNA, 20 nt, Homo sapiens
  source 1..20: mol_type = mRNA
aagcgcctggcagtgtacca

SEQ ID NO: 280 - RNA, 20 nt, Homo sapiens
  source 1..20: mol_type = mRNA
tggacgaggtgaaggagcag

SEQ ID NO: 281 - RNA, 20 nt, Homo sapiens
  source 1..20: mol_type = mRNA
acgaggtgaaggagcaggtg

SEQ ID NO: 282 - RNA, 20 nt, Homo sapiens
  source 1..20: mol_type = mRNA
cgcctcaagagctggttcga

SEQ ID NO: 283 - RNA, 20 nt, Homo sapiens
  source 1..20: mol_type = mRNA
gcctcaagagctggttcgag

SEQ ID NO: 284 - RNA, 20 nt, Homo sapiens
  source 1..20: mol_type = mRNA
cctcaagagctggttcgagc

SEQ ID NO: 285 - RNA, 20 nt, Homo sapiens
  source 1..20: mol_type = mRNA
ctcaagagctggttcgagcc

SEQ ID NO: 286 - RNA, 20 nt, Homo sapiens
  source 1..20: mol_type = mRNA
tcaagagctggttcgagccc

SEQ ID NO: 287 - RNA, 20 nt, Homo sapiens
  source 1..20: mol_type = mRNA
cctgtgcccagcgacaatca

SEQ ID NO: 288 - RNA, 20 nt, Homo sapiens
  source 1..20: mol_type = mRNA
ctgtgcccagcgacaatcac

SEQ ID NO: 289 - RNA, 20 nt, Homo sapiens
  source 1..20: mol_type = mRNA
tgtgcccagcgacaatcact

SEQ ID NO: 290 - RNA, 20 nt, Homo sapiens
  source 1..20: mol_type = mRNA
tgcccagcgacaatcactga

SEQ ID NO: 291 - RNA, 20 nt, Homo sapiens
  source 1..20: mol_type = mRNA
cccagcgacaatcactgaac

SEQ ID NO: 292 - RNA, 20 nt, Homo sapiens
  source 1..20: mol_type = mRNA
ccagcgacaatcactgaacg

SEQ ID NO: 293 - RNA, 20 nt, Homo sapiens
  source 1..20: mol_type = mRNA
cagcgacaatcactgaacgc

SEQ ID NO: 294 - RNA, 20 nt, Homo sapiens
  source 1..20: mol_type = mRNA
agcgacaatcactgaacgcc

SEQ ID NO: 295 - RNA, 20 nt, Homo sapiens
  source 1..20: mol_type = mRNA
gcgacaatcactgaacgccg

SEQ ID NO: 296 - RNA, 20 nt, Homo sapiens
  source 1..20: mol_type = mRNA
cgacaatcactgaacgccga

SEQ ID NO: 297 - RNA, 20 nt, Homo sapiens
  source 1..20: mol_type = mRNA
gacaatcactgaacgccgaa

SEQ ID NO: 298 - RNA, 20 nt, Homo sapiens
  source 1..20: mol_type = mRNA
acaatcactgaacgccgaag

SEQ ID NO: 299 - RNA, 20 nt, Homo sapiens
  source 1..20: mol_type = mRNA
caatcactgaacgccgaagc

SEQ ID NO: 300 - RNA, 20 nt, Homo sapiens
  source 1..20: mol_type = mRNA
atcactgaacgccgaagcct

SEQ ID NO: 301 - RNA, 20 nt, Homo sapiens
  source 1..20: mol_type = mRNA
tcactgaacgccgaagcctg

SEQ ID NO: 302 - RNA, 20 nt, Homo sapiens
  source 1..20: mol_type = mRNA
actgaacgccgaagcctgca

SEQ ID NO: 303 - RNA, 20 nt, Homo sapiens
  source 1..20: mol_type = mRNA
tcctggggtggaccctagtt

SEQ ID NO: 304 - RNA, 20 nt, Homo sapiens
  source 1..20: mol_type = mRNA
ctggggtggaccctagttta

SEQ ID NO: 305 - RNA, 20 nt, Homo sapiens
  source 1..20: mol_type = mRNA
tggggtggaccctagtttaa

SEQ ID NO: 306 - RNA, 20 nt, Homo sapiens
  source 1..20: mol_type = mRNA
aagattcaccaagtttcacg

SEQ ID NO: 307 - RNA, 20 nt, Homo sapiens
  source 1..20: mol_type = mRNA
agattcaccaagtttcacgc

SEQ ID NO: 308 - RNA, 20 nt, Homo sapiens
  source 1..20: mol_type = mRNA
gattcaccaagtttcacgca

SEQ ID NO: 309 - RNA, 18 nt, Homo sapiens
  source 1..18: mol_type = mRNA
ggaggacgtgcgcggccg

SEQ ID NO: 310 - DNA, 16 bp, Homo sapiens
  source 1..16: mol_type = genomic DNA
cggtgagaagcgcagt

SEQ ID NO: 311 - DNA, 16 bp, Homo sapiens
  source 1..16: mol_type = genomic DNA
ctagaaagagctggga

SEQ ID NO: 312 - DNA, 16 bp, Homo sapiens
  source 1..16: mol_type = genomic DNA
aggtagtctcaggaga

SEQ ID NO: 313 - DNA, 16 bp, Homo sapiens
  source 1..16: mol_type = genomic DNA
ctggactgggatgtaa

SEQ ID NO: 314 - DNA, 16 bp, Homo sapiens
  source 1..16: mol_type = genomic DNA
tggactgggatgtaag

SEQ ID NO: 315 - DNA, 16 bp, Homo sapiens
  source 1..16: mol_type = genomic DNA
actgggatgtaagcca

SEQ ID NO: 316 - DNA, 16 bp, Homo sapiens
  source 1..16: mol_type = genomic DNA
ctgggatgtaagccat

SEQ ID NO: 317 - DNA, 16 bp, Homo sapiens
  source 1..16: mol_type = genomic DNA
gggatgtaagccatag

SEQ ID NO: 318 - DNA, 16 bp, Homo sapiens
  source 1..16: mol_type = genomic DNA
ggatgtaagccatagc

SEQ ID NO: 319 - DNA, 16 bp, Homo sapiens
  source 1..16: mol_type = genomic DNA
tccacgagttgtcact

SEQ ID NO: 320 - DNA, 16 bp, Homo sapiens
  source 1..16: mol_type = genomic DNA
ccacgagttgtcacta

SEQ ID NO: 321 - DNA, 16 bp, Homo sapiens
  source 1..16: mol_type = genomic DNA
cgagttgtcactatca

SEQ ID NO: 322 - DNA, 16 bp, Homo sapiens
  source 1..16: mol_type = genomic DNA
tatcgagcacctactg

SEQ ID NO: 323 - DNA, 16 bp, Homo sapiens
  source 1..16: mol_type = genomic DNA
cagtgtcctcagatct

SEQ ID NO: 324 - DNA, 16 bp, Homo sapiens
  source 1..16: mol_type = genomic DNA
agtgtcctcagatctc

SEQ ID NO: 325 - DNA, 16 bp, Homo sapiens
  source 1..16: mol_type = genomic DNA
tgtcctcagatctcca

SEQ ID NO: 326 - DNA, 16 bp, Homo sapiens
  source 1..16: mol_type = genomic DNA
gtcctcagatctccat

SEQ ID NO: 327 - DNA, 16 bp, Homo sapiens
  source 1..16: mol_type = genomic DNA
gatctccataactggg

SEQ ID NO: 328 - DNA, 16 bp, Homo sapiens
  source 1..16: mol_type = genomic DNA
cagcgacacggtagct

SEQ ID NO: 329 - DNA, 16 bp, Homo sapiens
  source 1..16: mol_type = genomic DNA
agcgacacggtagcta

SEQ ID NO: 330 - DNA, 16 bp, Homo sapiens
  source 1..16: mol_type = genomic DNA
gacacggtagctagcc

SEQ ID NO: 331 - DNA, 16 bp, Homo sapiens
  source 1..16: mol_type = genomic DNA
gtagctagccgtcgat

SEQ ID NO: 332 - DNA, 16 bp, Homo sapiens
  source 1..16: mol_type = genomic DNA
tagctagccgtcgatt

SEQ ID NO: 333 - DNA, 16 bp, Homo sapiens
  source 1..16: mol_type = genomic DNA
agctagccgtcgattg

SEQ ID NO: 334 - DNA, 16 bp, Homo sapiens
  source 1..16: mol_type = genomic DNA
ctagccgtcgattgga

SEQ ID NO: 335 - DNA, 16 bp, Homo sapiens
  source 1..16: mol_type = genomic DNA
cgtcgattggagaact

SEQ ID NO: 336 - DNA, 16 bp, Homo sapiens
  source 1..16: mol_type = genomic DNA
gtcgattggagaactt

SEQ ID NO: 337 - DNA, 16 bp, Homo sapiens
  source 1..16: mol_type = genomic DNA
taaatggaacacggcg

SEQ ID NO: 338 - DNA, 16 bp, Homo sapiens
  source 1..16: mol_type = genomic DNA
tggaacacggcgctta

SEQ ID NO: 339 - DNA, 16 bp, Homo sapiens
  source 1..16: mol_type = genomic DNA
ggaacacggcgcttaa

SEQ ID NO: 340 - DNA, 16 bp, Homo sapiens
  source 1..16: mol_type = genomic DNA
gaacacggcgcttaac

SEQ ID NO: 341 - DNA, 16 bp, Homo sapiens
  source 1..16: mol_type = genomic DNA
aacacggcgcttaact

SEQ ID NO: 342 - DNA, 16 bp, Homo sapiens
  source 1..16: mol_type = genomic DNA
acacggcgcttaactg

SEQ ID NO: 343 - DNA, 16 bp, Homo sapiens
  source 1..16: mol_type = genomic DNA
acggcgcttaactgtg

SEQ ID NO: 344 - DNA, 16 bp, Homo sapiens
  source 1..16: mol_type = genomic DNA
cggcgcttaactgtga

SEQ ID NO: 345 - DNA, 16 bp, Homo sapiens
  source 1..16: mol_type = genomic DNA
ggcgcttaactgtgag

SEQ ID NO: 346 - DNA, 16 bp, Homo sapiens
  source 1..16: mol_type = genomic DNA
gcgcttaactgtgagg

SEQ ID NO: 347 - DNA, 16 bp, Homo sapiens
  source 1..16: mol_type = genomic DNA
cgcttaactgtgaggt

SEQ ID NO: 348 - DNA, 16 bp, Homo sapiens
  source 1..16: mol_type = genomic DNA
cttaactgtgaggttg

SEQ ID NO: 349 - DNA, 16 bp, Homo sapiens
  source 1..16: mol_type = genomic DNA
aactgtgaggttggag

SEQ ID NO: 350 - DNA, 16 bp, Homo sapiens
  source 1..16: mol_type = genomic DNA
tgtgaggttggagctt

SEQ ID NO: 351 - DNA, 16 bp, Homo sapiens
  source 1..16: mol_type = genomic DNA
gtgaggttggagctta

SEQ ID NO: 352 - DNA, 16 bp, Homo sapiens
  source 1..16: mol_type = genomic DNA
tgaggttggagcttag

SEQ ID NO: 353 - DNA, 16 bp, Homo sapiens
  source 1..16: mol_type = genomic DNA
gaggttggagcttaga

SEQ ID NO: 354 - DNA, 16 bp, Homo sapiens
  source 1..16: mol_type = genomic DNA
gaagggagaatgagga

SEQ ID NO: 355 - DNA, 16 bp, Homo sapiens
  source 1..16: mol_type = genomic DNA
gggagaatgaggaatg

SEQ ID NO: 356 - DNA, 16 bp, Homo sapiens
  source 1..16: mol_type = genomic DNA
ggagaatgaggaatgc

SEQ ID NO: 357 - DNA, 16 bp, Homo sapiens
  source 1..16: mol_type = genomic DNA
gagaatgaggaatgcg

SEQ ID NO: 358 - DNA, 16 bp, Homo sapiens
  source 1..16: mol_type = genomic DNA
gaatgaggaatgcgag

SEQ ID NO: 359 - DNA, 16 bp, Homo sapiens
  source 1..16: mol_type = genomic DNA
ttctatggaggccgac

SEQ ID NO: 360 - DNA, 16 bp, Homo sapiens
  source 1..16: mol_type = genomic DNA
cctcactggcggttga

SEQ ID NO: 361 - DNA, 16 bp, Homo sapiens
  source 1..16: mol_type = genomic DNA
ctcactggcggttgat

SEQ ID NO: 362 - DNA, 16 bp, Homo sapiens
  source 1..16: mol_type = genomic DNA
acagtttctccttccc

SEQ ID NO: 363 - DNA, 16 bp, Homo sapiens
  source 1..16: mol_type = genomic DNA
tctcatcctcacctca

SEQ ID NO: 364 - DNA, 16 bp, Homo sapiens
  source 1..16: mol_type = genomic DNA
ctcatcctcacctcaa

SEQ ID NO: 365 - DNA, 16 bp, Homo sapiens
  source 1..16: mol_type = genomic DNA
tcatcctcacctcaac

SEQ ID NO: 366 - DNA, 16 bp, Homo sapiens
  source 1..16: mol_type = genomic DNA
atcctcacctcaacct

SEQ ID NO: 367 - DNA, 16 bp, Homo sapiens
  source 1..16: mol_type = genomic DNA
ctcttctgaggcttct

SEQ ID NO: 368 - DNA, 16 bp, Homo sapiens
  source 1..16: mol_type = genomic DNA
tcttctgaggcttctg

SEQ ID NO: 369 - DNA, 16 bp, Homo sapiens
  source 1..16: mol_type = genomic DNA
cttctgaggcttctgt

SEQ ID NO: 370 - DNA, 16 bp, Homo sapiens
  source 1..16: mol_type = genomic DNA
ttctgaggcttctgtg

SEQ ID NO: 371 - DNA, 16 bp, Homo sapiens
  source 1..16: mol_type = genomic DNA
gcgatttgacgctctc

SEQ ID NO: 372 - DNA, 16 bp, Homo sapiens
  source 1..16: mol_type = genomic DNA
cgatttgacgctctct

SEQ ID NO: 373 - DNA, 16 bp, Homo sapiens
  source 1..16: mol_type = genomic DNA
gatttgacgctctctg

SEQ ID NO: 374 - DNA, 16 bp, Homo sapiens
  source 1..16: mol_type = genomic DNA
atttgacgctctctgg

SEQ ID NO: 375 - DNA, 16 bp, Homo sapiens
  source 1..16: mol_type = genomic DNA
tttgacgctctctggg

SEQ ID NO: 376 - DNA, 16 bp, Homo sapiens
  source 1..16: mol_type = genomic DNA
cccatccttgagatag

SEQ ID NO: 377 - DNA, 16 bp, Homo sapiens
  source 1..16: mol_type = genomic DNA
ccatccttgagatagg

SEQ ID NO: 378 - DNA, 16 bp, Homo sapiens
  source 1..16: mol_type = genomic DNA
atctgcccgtttcgat

SEQ ID NO: 379 - DNA, 16 bp, Homo sapiens
  source 1..16: mol_type = genomic DNA
tctgcccgtttcgatc

SEQ ID NO: 380 - DNA, 16 bp, Homo sapiens
  source 1..16: mol_type = genomic DNA
ctgcccgtttcgatct

SEQ ID NO: 381 - DNA, 16 bp, Homo sapiens
  source 1..16: mol_type = genomic DNA
tgcccgtttcgatctc

SEQ ID NO: 382 - DNA, 16 bp, Homo sapiens
  source 1..16: mol_type = genomic DNA
gcccgtttcgatctcc

SEQ ID NO: 383 - DNA, 16 bp, Homo sapiens
  source 1..16: mol_type = genomic DNA
ccgtttcgatctccca

SEQ ID NO: 384 - DNA, 16 bp, Homo sapiens
  source 1..16: mol_type = genomic DNA
cgtttcgatctcccaa

SEQ ID NO: 385 - DNA, 16 bp, Homo sapiens
  source 1..16: mol_type = genomic DNA
gtttcgatctcccaaa

SEQ ID NO: 386 - DNA, 16 bp, Homo sapiens
  source 1..16: mol_type = genomic DNA
tttcgatctcccaaag

SEQ ID NO: 387 - DNA, 16 bp, Homo sapiens
  source 1..16: mol_type = genomic DNA
ctgggagttagaggtt

SEQ ID NO: 388 - DNA, 16 bp, Homo sapiens
  source 1..16: mol_type = genomic DNA
gtgaataccagacacg

SEQ ID NO: 389 - DNA, 16 bp, Homo sapiens
  source 1..16: mol_type = genomic DNA
tgaataccagacacgg

SEQ ID NO: 390 - DNA, 16 bp, Homo sapiens
  source 1..16: mol_type = genomic DNA
cacacaaggacactca

SEQ ID NO: 391 - DNA, 16 bp, Homo sapiens
  source 1..16: mol_type = genomic DNA
ccagaaggtcaaggtt

SEQ ID NO: 392 - DNA, 16 bp, Homo sapiens
  source 1..16: mol_type = genomic DNA
cagaaggtcaaggttg

SEQ ID NO: 393 - DNA, 16 bp, Homo sapiens
  source 1..16: mol_type = genomic DNA
agaaggtcaaggttgc

SEQ ID NO: 394 - DNA, 16 bp, Homo sapiens
  source 1..16: mol_type = genomic DNA
gaaggtcaaggttgca

SEQ ID NO: 395 - DNA, 16 bp, Homo sapiens
  source 1..16: mol_type = genomic DNA
gaaccatgttcaggcc

SEQ ID NO: 396 - DNA, 16 bp, Homo sapiens
  source 1..16: mol_type = genomic DNA
gtgattaaaccgactc

SEQ ID NO: 397 - DNA, 16 bp, Homo sapiens
  source 1..16: mol_type = genomic DNA
accatggctccaaaga

SEQ ID NO: 398 - DNA, 16 bp, Homo sapiens
  source 1..16: mol_type = genomic DNA
ccatggctccaaagaa

SEQ ID NO: 399 - DNA, 16 bp, Homo sapiens
  source 1..16: mol_type = genomic DNA
catggctccaaagaag

SEQ ID NO: 400 - DNA, 16 bp, Homo sapiens
  source 1..16: mol_type = genomic DNA
tttgtggagcaccttc

SEQ ID NO: 401 - DNA, 16 bp, Homo sapiens
  source 1..16: mol_type = genomic DNA
ccctaggtactagatg

SEQ ID NO: 402 - DNA, 16 bp, Homo sapiens
  source 1..16: mol_type = genomic DNA
cctaggtactagatgc

SEQ ID NO: 403 - DNA, 16 bp, Homo sapiens
  source 1..16: mol_type = genomic DNA
ctaggtactagatgcc

SEQ ID NO: 404 - DNA, 16 bp, Homo sapiens
  source 1..16: mol_type = genomic DNA
aggtactagatgcctg

SEQ ID NO: 405 - DNA, 16 bp, Homo sapiens
  source 1..16: mol_type = genomic DNA
gtactagatgcctgga

SEQ ID NO: 406 - DNA, 16 bp, Homo sapiens
  source 1..16: mol_type = genomic DNA
tactagatgcctggac

SEQ ID NO: 407 - DNA, 16 bp, Homo sapiens
  source 1..16: mol_type = genomic DNA
actagatgcctggacg

SEQ ID NO: 408 - DNA, 16 bp, Homo sapiens
  source 1..16: mol_type = genomic DNA
tcagaaggaccctgac

SEQ ID NO: 409 - DNA, 16 bp, Homo sapiens
  source 1..16: mol_type = genomic DNA
acttgttccacacagg

SEQ ID NO: 410 - DNA, 16 bp, Homo sapiens
  source 1..16: mol_type = genomic DNA
cttgttccacacagga

SEQ ID NO: 411 - DNA, 16 bp, Homo sapiens
  source 1..16: mol_type = genomic DNA
caagcggtggagacag

SEQ ID NO: 412 - DNA, 16 bp, Homo sapiens
  source 1..16: mol_type = genomic DNA
aggaactgaggtgagt

SEQ ID NO: 413 - DNA, 16 bp, Homo sapiens
  source 1..16: mol_type = genomic DNA
gaactgaggtgagtgt

SEQ ID NO: 414 - DNA, 16 bp, Homo sapiens
  source 1..16: mol_type = genomic DNA
aactgaggtgagtgtc

SEQ ID NO: 415 - DNA, 16 bp, Homo sapiens
  source 1..16: mol_type = genomic DNA
caggtccaggtttcat

SEQ ID NO: 416 - DNA, 16 bp, Homo sapiens
  source 1..16: mol_type = genomic DNA
cctgtcgctaagtctt

SEQ ID NO: 417 - DNA, 16 bp, Homo sapiens
  source 1..16: mol_type = genomic DNA
ctgtcgctaagtcttg

SEQ ID NO: 418 - DNA, 16 bp, Homo sapiens
  source 1..16: mol_type = genomic DNA
tgtcgctaagtcttgg

SEQ ID NO: 419 - DNA, 16 bp, Homo sapiens
  source 1..16: mol_type = genomic DNA
gtcgctaagtcttggg

SEQ ID NO: 420 - DNA, 16 bp, Homo sapiens
  source 1..16: mol_type = genomic DNA
gtctctgctggttcta

SEQ ID NO: 421 - DNA, 16 bp, Homo sapiens
  source 1..16: mol_type = genomic DNA
tctctgctggttctag

SEQ ID NO: 422 - DNA, 16 bp, Homo sapiens
  source 1..16: mol_type = genomic DNA
cttcccttctgactca

SEQ ID NO: 423 - DNA, 16 bp, Homo sapiens
  source 1..16: mol_type = genomic DNA
ttcccttctgactcag

SEQ ID NO: 424 - DNA, 16 bp, Homo sapiens
  source 1..16: mol_type = genomic DNA
ctgactcagtctctca

SEQ ID NO: 425 - DNA, 16 bp, Homo sapiens
  source 1..16: mol_type = genomic DNA
tgactcagtctctcac

SEQ ID NO: 426 - DNA, 16 bp, Homo sapiens
  source 1..16: mol_type = genomic DNA
gactcagtctctcaca

SEQ ID NO: 427 - DNA, 16 bp, Homo sapiens
  source 1..16: mol_type = genomic DNA
actcagtctctcacac

SEQ ID NO: 428 - DNA, 16 bp, Homo sapiens
  source 1..16: mol_type = genomic DNA
ctcagtctctcacact

SEQ ID NO: 429 - DNA, 16 bp, Homo sapiens
  source 1..16: mol_type = genomic DNA
tcagtctctcacactc

SEQ ID NO: 430 - DNA, 16 bp, Homo sapiens
  source 1..16: mol_type = genomic DNA
agtctctcacactcgt

SEQ ID NO: 431 - DNA, 16 bp, Homo sapiens
  source 1..16: mol_type = genomic DNA
gtctctcacactcgtc

SEQ ID NO: 432 - DNA, 16 bp, Homo sapiens
  source 1..16: mol_type = genomic DNA
tctctcacactcgtcc

SEQ ID NO: 433 - DNA, 16 bp, Homo sapiens
  source 1..16: mol_type = genomic DNA
ctcccactgtgcgaca

SEQ ID NO: 434 - DNA, 16 bp, Homo sapiens
  source 1..16: mol_type = genomic DNA
tcccactgtgcgacac

SEQ ID NO: 435 - DNA, 16 bp, Homo sapiens
  source 1..16: mol_type = genomic DNA
actgtgcgacaccctc

SEQ ID NO: 436 - DNA, 16 bp, Homo sapiens
  source 1..16: mol_type = genomic DNA
tgatggacgagaccat

SEQ ID NO: 437 - DNA, 16 bp, Homo sapiens
  source 1..16: mol_type = genomic DNA
tacaaatcggaactgg

SEQ ID NO: 438 - DNA, 16 bp, Homo sapiens
  source 1..16: mol_type = genomic DNA
caaatcggaactggag

SEQ ID NO: 439 - DNA, 16 bp, Homo sapiens
  source 1..16: mol_type = genomic DNA
aaatcggaactggagg

SEQ ID NO: 440 - DNA, 16 bp, Homo sapiens
  source 1..16: mol_type = genomic DNA
aatcggaactggagga

SEQ ID NO: 441 - DNA, 16 bp, Homo sapiens
  source 1..16: mol_type = genomic DNA
atcggaactggaggaa

SEQ ID NO: 442 - DNA, 16 bp, Homo sapiens
  source 1..16: mol_type = genomic DNA
tcggaactggaggaac

SEQ ID NO: 443 - DNA, 16 bp, Homo sapiens
  source 1..16: mol_type = genomic DNA
tcaagagctggttcga

SEQ ID NO: 444 - DNA, 16 bp, Homo sapiens
  source 1..16: mol_type = genomic DNA
agcgacaatcactgaa

SEQ ID NO: 445 - DNA, 16 bp, Homo sapiens
  source 1..16: mol_type = genomic DNA
gcgacaatcactgaac

SEQ ID NO: 446 - DNA, 16 bp, Homo sapiens
  source 1..16: mol_type = genomic DNA
cgacaatcactgaacg

SEQ ID NO: 447 - DNA, 16 bp, Homo sapiens
  source 1..16: mol_type = genomic DNA
gacaatcactgaacgc

SEQ ID NO: 448 - DNA, 16 bp, Homo sapiens
  source 1..16: mol_type = genomic DNA
acaatcactgaacgcc

SEQ ID NO: 449 - DNA, 16 bp, Homo sapiens
  source 1..16: mol_type = genomic DNA
aatcactgaacgccga

SEQ ID NO: 450 - DNA, 16 bp, Homo sapiens
  source 1..16: mol_type = genomic DNA
atcactgaacgccgaa

SEQ ID NO: 451 - DNA, 16 bp, Homo sapiens
  source 1..16: mol_type = genomic DNA
tcactgaacgccgaag

SEQ ID NO: 452 - DNA, 16 bp, Homo sapiens
  source 1..16: mol_type = genomic DNA
tgaacgccgaagcctg

SEQ ID NO: 453 - DNA, 16 bp, Homo sapiens
  source 1..16: mol_type = genomic DNA
aacgccgaagcctgca

SEQ ID NO: 454 - DNA, 16 bp, Homo sapiens
  source 1..16: mol_type = genomic DNA
tcaccaagtttcacgc

SEQ ID NO: 455 - DNA, 16 bp, Homo sapiens
  source 1..16: mol_type = genomic DNA
caccaagtttcacgca